(19) United States
(12) Patent Application Publication
(10) Pub. No.: US 2022/0341959 A1
(43) Pub. Date: Oct. 27, 2022

(54) DEVICES AND METHODS FOR LOADING OF FLUIDIC RECEPTACLES

(71) Applicant: Quantum-Si Incorporated, Guilford, CT (US)

(72) Inventors: Xiaoxiao Ma, Branford, CT (US); Jonathan C. Schultz, Guilford, CT (US); Caixia Lv, Guilford, CT (US); Brittany Lathrop, Derby, CT (US)

(73) Assignee: Quantum-Si Incorporated, Guilford, CT (US)

(21) Appl. No.: 17/725,127

(22) Filed: Apr. 20, 2022

Related U.S. Application Data
(60) Provisional application No. 63/271,944, filed on Oct. 26, 2021.
(60) Provisional application No. 63/177,882, filed on Apr. 21, 2021.

(51) Int. Cl. G01N 35/10; C12Q 1/6874; B01L 3/00

(52) U.S. Cl. CPC G01N 35/1011; C12Q 1/6874; B01L 3/502761; B01L 3/502715; B01L 2300/0663; B01L 2300/0829; B01L 2400/0487

(57) ABSTRACT

Aspects of this disclosure relate to systems, devices, and methods for the transfer of fluid to fluidic receptacles. In some embodiments, the fluid contains one or more molecules (e.g., one or more peptides, proteins, and/or nucleic acids) of interest, and the fluidic receptacle includes an integrated device. In certain embodiments, the one or more molecules can be immobilized on the integrated device for subsequent analysis (e.g., sequencing). Certain aspects of the present disclosure are directed towards systems and methods that can, in some instances, enhance the immobilization of the one or more molecules on the integrated device, e.g., by improving a rate of sample interaction with the integrated device. Through the use of systems, devices, and methods of the instant disclosure, target molecules may be more readily sequenced or prepared for sequencing. For example, in some embodiments, systems, devices, and methods of the instant disclosure allow automated loading of the fluidic receptacle using a fluidic device.

### RELATED APPLICATIONS

This application claims priority under 35 U.S.C. § 119(e) to U.S. Provisional Application No. 63/177,882, filed Apr. 21, 2021, and entitled “DEVICES AND METHODS FOR LOADING OF FLUIDIC RECEPTACLES,” and to U.S. Provisional Application No. 63/271,944, filed Oct. 26, 2021, and entitled “DEVICES AND METHODS FOR LOADING OF FLUIDIC RECEPTACLES,” which are incorporated herein by reference in their entirety for all purposes.

### TECHNICAL FIELD

Methods, articles, and systems related to manipulation and/or preparation of fluids, are generally described.

### BACKGROUND

Analysis of biopolymers has emerged as an important tool in the study of biological systems. These analyses of an individual organism or sample type can provide insights into cellular processes and response patterns, which lead to improved diagnostic and therapeutic strategies. The complexity surrounding biopolymer compositions and modification present challenges in determining large-scale sequencing information for a biological sample.

Improved and more convenient techniques and systems for manipulating (e.g., preparing) biopolymer compositions are desirable.

### SUMMARY

Aspects of this disclosure relate to systems, devices, and methods for the transfer of fluid to fluidic receptacles. In some embodiments, the fluid comprises one or more molecules (e.g., one or more peptides, proteins, and/or nucleic acids) of interest, and the fluidic receptacle comprises an integrated device. In certain embodiments, the one or more molecules (e.g., from a sample) can be immobilized on the integrated device for subsequent analysis (e.g., sequencing). Certain aspects of the present disclosure are directed towards systems and methods that can, in some instances, enhance the immobilization of the one or more molecules (e.g., peptides) on the integrated device, e.g., by improving a rate of sample interaction with the integrated device. Through the use of systems, devices, and methods of the instant disclosure, target molecules may be more readily sequenced or prepared for sequencing. For example, in some embodiments, systems, devices, and methods of the instant disclosure allow automated loading of the fluidic receptacle using a fluidic device. The subject matter of the present invention involves, in some cases, interrelated products, alternative solutions to a particular problem, and/or a plurality of different uses of one or more systems and/or articles.

In one aspect, a fluidic system for transferring fluid to a fluidic receptacle is provided. In some embodiments, the fluidic system for transferring fluid to a fluidic receptacle comprises: a fluidic device, comprising: a first alignment feature; a channel that is fluidically connected to the first alignment feature, and a second alignment feature; wherein, the fluidic device is configured to receive the fluidic receptacle in an alignment with respect to the fluidic device via a first coupling between the first alignment feature and a third alignment feature, located on the fluidic receptacle, and via a second coupling between the second alignment feature and a fourth alignment feature, located on the fluidic receptacle, such that the channel is fluidically connected to the fluidic receptacle through the first alignment feature and the second alignment feature.

In another aspect, a method is provided. In some embodiments, the method comprises: coupling a fluidic receptacle to two or more alignment features; transferring a fluid to the fluidic receptacle via at least one of the alignment features; and decoupling the fluidic receptacle from the alignment features.

In still another aspect, a method is provided. In some embodiments, the method comprises: aligning a fluidic device by coupling two or more alignment features; and transferring a fluid through at least one of the alignment features.

In some embodiments, the method comprises: aligning a fluidic device with a fluidic receptacle by coupling two or more alignment features of the fluidic device to a fluidic receptacle; and transferring a fluid through at least one of the alignment features.

In yet another aspect, a fluidic device is provided. In some embodiments, the fluidic device comprises: a solid substrate, a channel, and a mounting element within or mechanically coupled to the solid substrate, the mounting element having a surface angled with respect to a lateral dimension of the fluidic device; wherein: the fluidic device is configured to receive a fluidic receptacle into the mounting element, such that the channel is fluidically connected to the fluidic receptacle, and an angle between the surface and the lateral dimension of the fluidic device is greater than or equal to 10° and less than or equal to 80°. In some embodiments, the mounting element comprises a recess within the solid substrate.

In one aspect, a method of mixing is provided. In some embodiments, the method of mixing comprises: translating a roller across at least a portion of a surface of a channel containing a quantity of fluid in a first direction while applying pressure to the surface; and translating the roller across at least a portion of the surface in a second, different direction while applying pressure to at least a portion of the surface, wherein the applied pressure is maintained between the translating the roller in the first direction and the translating the roller in the second direction.

In another aspect, a method is provided. According to some embodiments, the method comprises: transferring, via peristaltic pumping, a first quantity of a fluid into a fluidic receptacle, the fluidic receptacle having a volume exceeding a volume of the quantity of fluid; mixing the fluid in the fluidic receptacle for a first time; transferring, via peristaltic pumping, a second quantity of the fluid into the fluidic receptacle; and mixing the fluid in the fluidic receptacle for a second time.

In yet another aspect, a method of making a fluidic device is provided. According to some embodiments, the method of making the fluidic device comprises: assembling a surface article comprising a surface layer with a base layer to form a cartridge of the fluidic device, wherein: the surface layer comprises an elastomer, the base layer comprises one or more channels, and fluidically connecting at least one of the channels to a discrete fluidic device portion comprising an alignment feature. In some embodiments, at least some of the one or more channels have a substantially triangularly-shaped cross-section.

In still another aspect, a method is provided. According to certain embodiments, the method comprises: assembling a fluidic system for transferring fluid to a fluidic receptacle by receiving the fluidic receptacle into a fluidic device in an alignment with respect to the fluidic device by: coupling a first alignment feature of the fluidic device to a third alignment feature, located on the fluidic receptacle; coupling a second alignment feature of the fluidic device to a fourth alignment feature, located on the fluidic receptacle; and forming a channel that, upon assembly of the fluidic system, is fluidically connected to the fluidic receptacle through first alignment feature and the second alignment feature.

In another aspect, a method is provided. According to certain embodiments, the method comprises: assembling a first discrete fluidic device portion, comprising a relatively hard material, and a second discrete fluidic device portion, comprising a relatively soft material, into a discrete coupling portion of a fluidic device; and fluidically connecting an alignment feature of the first fluidic device portion and/or the second fluidic device portion to a cartridge of a fluidic device, wherein the fluidic device is at least partially formed by: forming one or more channels in a base layer of the cartridge; and attaching a surface layer that comprises an elastomer to the base layer of the cartridge. In some embodiments, at least some of the one or more channels have a substantially triangularly-shaped cross-section.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale unless otherwise indicated. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1A presents a cross-sectional schematic illustration of a fluidic device and a fluidic receptacle, according to certain embodiments;

FIG. 1B presents a cross-sectional schematic illustration of a fluidic device and a fluidic receptacle, according to certain embodiments;

FIG. 1C presents a cross-sectional schematic illustration of a fluidic device and a fluidic receptacle, according to certain embodiments;

FIG. 1D presents a cross-sectional schematic illustration of a fluidic device and a fluidic receptacle, according to certain embodiments;

FIG. 2 presents a cross-sectional schematic illustration of a sample well of an integrated device, according to certain embodiments;

FIG. 3 presents a cross-sectional schematic illustration of an integrated device, according to certain embodiments;

FIGS. 4A-4D show cross-sectional schematic illustrations of channels of a fluidic device, according to certain embodiments;

FIG. 5A shows a cross-sectional schematic illustration of translating a roller across a surface of a channel, according to certain embodiments;

FIG. 5B shows a cross-sectional schematic illustration of translating a roller across a surface of a channel, according to certain embodiments;

FIG. 6A shows a cross-sectional schematic illustration of transferring a fluid to a fluidic receptacle, according to certain embodiments;

FIG. 6B shows a cross-sectional schematic illustration of mixing fluid in a fluidic receptacle, according to certain embodiments;

FIG. 6C shows a cross-sectional schematic illustration of transferring a fluid to a fluidic receptacle, according to certain embodiments;

FIG. 6D shows a cross-sectional schematic illustration of mixing fluid in a fluidic receptacle, according to certain embodiments;

FIG. 7 shows an example scheme of peptide surface immobilization, according to certain embodiments;

FIG. 8 shows an exemplary fluidic system, according to certain embodiments;

FIG. 9A shows a schematic diagram of sample preparation device, according to certain embodiments;

FIG. 9B shows a schematic diagram of sample preparation device, according to certain embodiments;

FIG. 10A shows a non-limiting example of protein sequencing by iterative terminal amino acid detection and cleavage, according to certain embodiments;

FIG. 10B shows a non-limiting example of protein sequencing by iterative terminal amino acid detection and cleavage, according to certain embodiments;

FIG. 11A shows a perspective schematic illustration of a fluidic device for transferring fluid to a fluidic receptacle and a fluidic receptacle, according to certain embodiments;

FIG. 11B shows an exploded perspective schematic illustration of a fluidic device for transferring fluid to a fluidic receptacle and a fluidic receptacle, according to certain embodiments;

FIG. 11C shows a perspective schematic illustration of a substrate of a fluidic device for transferring fluid to a fluidic receptacle, according to certain embodiments;

FIG. 11D shows a side-view schematic illustration of a fluidic device for transferring fluid to a fluidic receptacle and a fluidic receptacle, according to certain embodiments;

FIG. 11E shows a top-view schematic illustration of a fluidic device for transferring fluid to a fluidic receptacle and a fluidic receptacle, according to certain embodiments;

FIG. 11F shows a bottom-view schematic illustration of a fluidic device for transferring fluid to a fluidic receptacle, according to certain embodiments;

FIG. 11G shows a bottom-view schematic illustration of a fluidic device without a seal plate for transferring fluid to a fluidic receptacle, according to certain embodiments;

FIG. 11H shows a perspective schematic illustration of a seal plate, according to certain embodiments;

FIG. 11I shows a perspective schematic illustration of a fluidic device portion, according to certain embodiments;

FIG. 11J shows a perspective schematic illustration of a fluidic device portion, according to certain embodiments;

FIG. 11K shows a perspective schematic illustration of a fluidic receptacle portion, according to certain embodiments;

FIG. 11L shows a perspective schematic illustration of a fluidic receptacle portion, according to certain embodiments;

FIG. 11M shows a perspective schematic illustration of a mechanical coupler, according to certain embodiments;

FIG. 11N shows a perspective schematic illustration of a mechanical coupler, according to certain embodiments;

FIG. 12A shows a perspective schematic illustration of a first fluidic device portion, a second fluidic device portion, and a fluidic receptacle, according to certain embodiments;

FIG. 12B shows a perspective schematic illustration of a first fluidic device portion and a second fluidic device portion, according to certain embodiments;

FIG. 12C shows a perspective schematic illustration of a first fluidic device portion and a second fluidic device portion, according to certain embodiments;

FIG. 12D shows a perspective schematic illustration of a first fluidic device portion, according to certain embodiments;

FIG. 12E shows a perspective schematic illustration of a first fluidic device portion, according to certain embodiments;

FIG. 12F shows a perspective schematic illustration of a second fluidic device portion, according to certain embodiments;

FIG. 12G shows a perspective schematic illustration of a second fluidic device portion, according to certain embodiments;

FIG. 12H shows a cross-sectional schematic illustration of a first fluidic device portion and a second fluidic device portion, according to certain embodiments;

FIG. 13A shows a perspective schematic illustration of a fluidic device for transferring fluid to a fluidic receptacle and a fluidic receptacle, according to certain embodiments;

FIG. 13B shows a side-view schematic illustration of a fluidic device for transferring fluid to a fluidic receptacle and a fluidic receptacle, according to certain embodiments;

FIG. 13C shows a bottom-view schematic illustration of a fluidic device for transferring fluid to a fluidic receptacle, according to certain embodiments;

FIGS. 13D-13E show various perspectives of an exemplary mounting element, fluidic receptacle, first fluidic device portion, and mechanical coupler, according to certain embodiments;

FIG. 13F shows a schematic, perspective illustration of an exemplary mounting element, fluidic receptacle, first fluidic device portion, and second fluidic device portion, according to certain embodiments;

FIG. 13G provides a schematic, perspective illustration of an exemplary mounting element, according to certain embodiments;

FIGS. 14A-14B provide perspective, schematic illustrations of a first fluidic device portion and a second fluidic device portion, according to certain embodiments;

FIG. 14C provides a perspective, schematic illustration of a truncated section of a first fluidic device portion and a second fluidic device portion, according to certain embodiments;

FIGS. 15A-15B show perspective illustrations of channels of fluidic devices, according to certain embodiments;

FIG. 16 shows a flow diagram of an exemplary method of loading a fluidic device, according to certain embodiments;

FIG. 17 shows a flow diagram of an exemplary method of loading a fluidic device, according to certain embodiments;

FIG. 18 shows a flow diagram of an exemplary method of loading a fluidic device, according to certain embodiments;

FIG. 19 shows a flow diagram of an exemplary method of loading a fluidic device, according to certain embodiments;

FIG. 20 shows an exemplary homogeneous cumulative recognition pulsing activity profile measured using an integrated device, according to certain embodiments

FIGS. 21A-21D show cross-sectional schematic illustrations of mixing of fluid within a fluidic receptacle, according to certain embodiments;

FIG. 22 presents exemplary homogeneous cumulative recognition pulsing activity profiles measured using exemplary integrated devices, according to certain embodiments;

FIG. 23 presents exemplary leak test results, according to certain embodiments;

FIG. 24 presents exemplary leak test results, according to certain embodiments;

FIGS. 25A-25B present loading fractions of exemplary fluidic devices, according to certain embodiments;

FIGS. 26A-26B present mappable reads of exemplary fluidic devices, according to certain embodiments;

FIG. 27 presents mappable reads of exemplary fluidic devices with a particular surface chemistry, according to certain embodiments;

FIG. 28 presents mappable reads of exemplary fluidic devices with a particular surface chemistry, according to certain embodiments; and

FIG. 29 presents mappable reads of exemplary fluidic devices with a particular surface chemistry, according to certain embodiments.

### DETAILED DESCRIPTION

In some aspects, this disclosure provides methods, devices, and systems for the transfer and analysis of fluids (e.g., samples comprising peptides and/or nucleotides). In some embodiments, a fluidic receptacle (e.g., an integrated device for sequencing molecules or interest) is fluidically connected to alignment features (e.g., alignment features of a fluidic device) that permit the transfer of fluid through the alignment features to the fluidic receptacle, while receiving and/or holding the fluidic receptacle in an alignment. The alignment can, in some embodiments, advantageously facilitate the separation of gas bubbles of the fluid from liquid (e.g., a sample) of the fluid, while providing ergonomic advantages. The sample may, for example, comprise one or more molecules such as peptides and/or nucleotides. In some instances, the one or more molecules of the sample are formed within a fluid. For example, the fluidic device described herein may be configured to receive the fluidic receptacle at an acute angle with respect to a lateral dimension of the fluidic device (e.g., a lateral dimension of a substrate of a fluidic device). In some aspects, advantageous methods of transferring fluid to and mixing fluid in fluidic receptacles are provided. According to some embodiments, apparatuses and methods described herein can advantageously improve the loading of a fluidic device, e.g., by increasing a proportion of a sample that binds to the fluidic device, and/or by allowing automated loading of the fluidic device. In one, non-limiting embodiment, the fluidic device is configured to load a fluid sample into a fluidic receptacle comprising an integrated device, so that the fluid sample can be subsequently analyzed (e.g., a target molecule within the fluid sample can be subsequently sequenced).

Workflows for the loading of samples (e.g., protein samples, nucleotide samples) into fluidic receptacles for analysis (e.g., sequencing) often result in the loss of a significant proportion of the samples. The sample may be lost, for example, as a consequence of dead volume in the system, bubbles in the sample, or insufficient agitation of the sample in the presence of a substrate. Approaches that reduce the loss of the sample during loading of the fluidic receptacle may provide numerous benefits such as greater loading efficiency, smaller required sample volumes, and/or a reduction in a quantity of a target molecule (e.g., a protein or a nucleic acid) required for reliable analysis.

Aspects of the present disclosure can, in some embodiments, reduce sample loss during the loading of fluidic receptacles. For example, in certain embodiments, a fluidic device described herein is configured to receive a fluidic receptacle in an alignment favorable for separating bubbles from liquid samples. In some embodiments, the fluidic device is configured to receive the fluidic receptacle in an alignment via alignment features, such that the fluidic device and the fluidic receptacle are fluidically coupled via the alignment features. The formation of a fluidic coupling via an alignment feature can, according to certain embodiments, result in a reduction in dead volume of the system. Additional aspects of the present disclosure are directed towards methods of agitating and loading samples that can, in certain embodiments, reduce sample loss without producing an undesirable net flow of material.

According to some aspects, a fluidic system is provided. The fluidic system, according to some embodiments, can be used for transferring fluid to a fluidic receptacle, e.g., using peristaltic pumping. According to some embodiments, the fluidic system comprises a fluidic device, as described herein, configured to receive a fluidic receptacle using alignment features. The fluidic device may comprise, for example, a cartridge, as described in more detail below. According to certain embodiments, the fluidic device is configured to be fluidically connected to the fluidic receptacle via the alignment features. This may allow the fluidic device to be simultaneously aligned with and fluidically connected to the fluidic receptacle. Connections of this variety advantageously reduce a dead volume of a fluidic connection between the fluidic device and the fluidic receptacle, (e.g., by reducing the need for excess channel length), according to certain embodiments.

In some embodiments, components of fluidic devices, articles, and systems described herein are fluidically connected. Two components are fluidically connected if, under some configurations of an embodiment, fluid may pass between them. For example, a first fluidic device component and a second fluidic device component may be fluidically connected if they are connected by a channel, a microchannel, or a tube. As another example, two components separated by a valve would still be considered fluidically connected, as long as the valve could be configured to permit fluid flow between the two components. In contrast, two components that are only connected mechanically, without a fluidic pathway between them, would not be considered to be fluidically connected. Fluidically connected components may be directly fluidically connected (i.e., connected by a fluidic pathway that does not pass through any intervening components). However, fluidically connected components may in some cases be connected by a fluidic pathway through 1, 2, 3, 4, 5, 8, 10, 15, 20, or more intervening components.

The fluidic device, according to some embodiments, comprises a channel. The channel, in some embodiments, is fluidically connected to an alignment feature of the fluidic device. The alignment feature may be a first alignment feature of the fluidic device. According to some embodiments, the fluidic device further comprises a second alignment feature. The second alignment feature is connected to the channel, according to certain embodiments, e.g., via a fluidic connection established by a fluidic pathway passing through a fluidic receptacle. However, in some embodiments or configurations, the second alignment feature is not connected to the channel. According to some embodiments, the fluidic device is configured to receive a fluidic receptacle. For example, the fluidic device is configured to hold the fluidic receptacle, according to some embodiments. The fluidic device comprises a cartridge, according to some embodiments. In some embodiments, the fluidic device comprises a solid substrate (e.g., a solid substrate of cartridge). The fluidic device may comprise a mounting element configured to receive the fluidic receptacle. For example, the mounting element may mechanically couple to a received fluidic receptacle. As another example, the mounting element may not mechanically couple to a received fluidic receptacle. For instance, the mounting element may be configured such that the fluidic receptacle rests in or against the mounting element. In some embodiments, the solid substrate of the fluidic device comprises a mounting element. For example, the solid substrate may comprise a recess configured to receive the fluidic receptacle. Exemplary mounting elements may comprise recesses, raised edges, friction-inducing features, clamps, clips, grips, rails, and/or any of a variety of other features capable of receiving the fluidic receptacle into the mounting element. For example, the mounting element may comprise a recess, because a fluidic receptacle can be localized within the recess such that it is held in position with respect to the solid substrate. As another example, a mounting element may comprise a raised edge, e.g., against which an edge of a fluidic receptacle may be localized, such that the fluidic receptacle is held in position with respect to the solid substrate. To provide yet another example, a mounting element may comprise a friction-inducing feature, such as a friction-inducing surface, onto which the fluidic device can be localized, such that the induced friction holds the fluidic receptacle in position with respect to the substrate. Combinations of these features are also possible. For instance, a mounting element may comprise a raised edge and a friction-inducing feature, such that the fluidic receptacle can be localized against both the raised edge and the friction-inducing feature, thereby holding the fluidic receptacle in position within the mounting element.

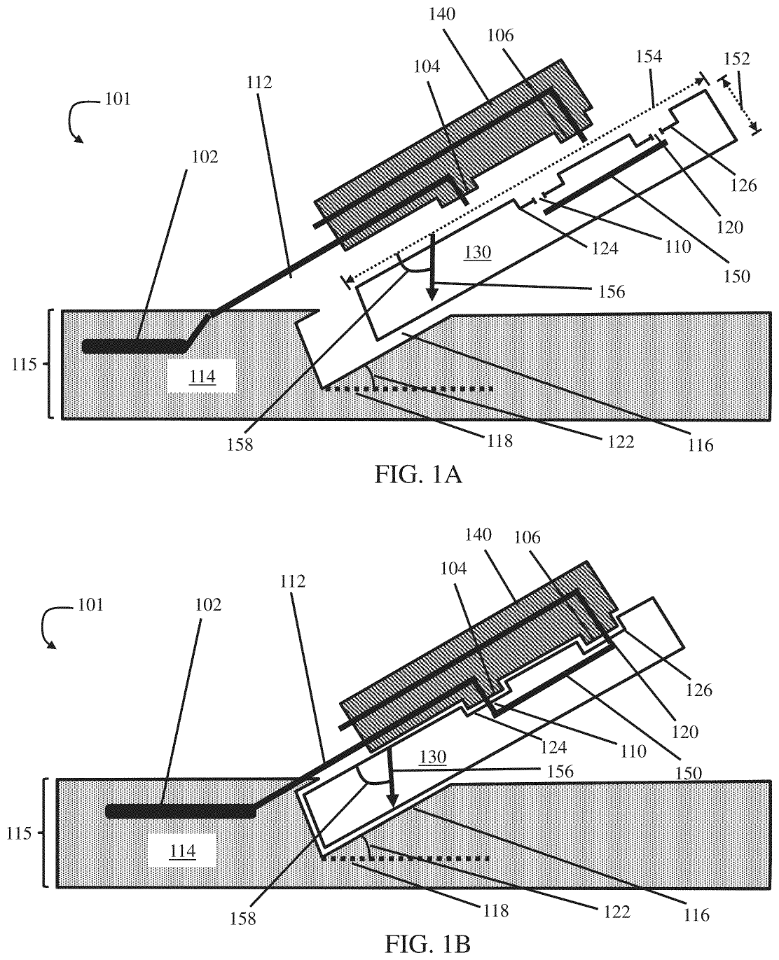

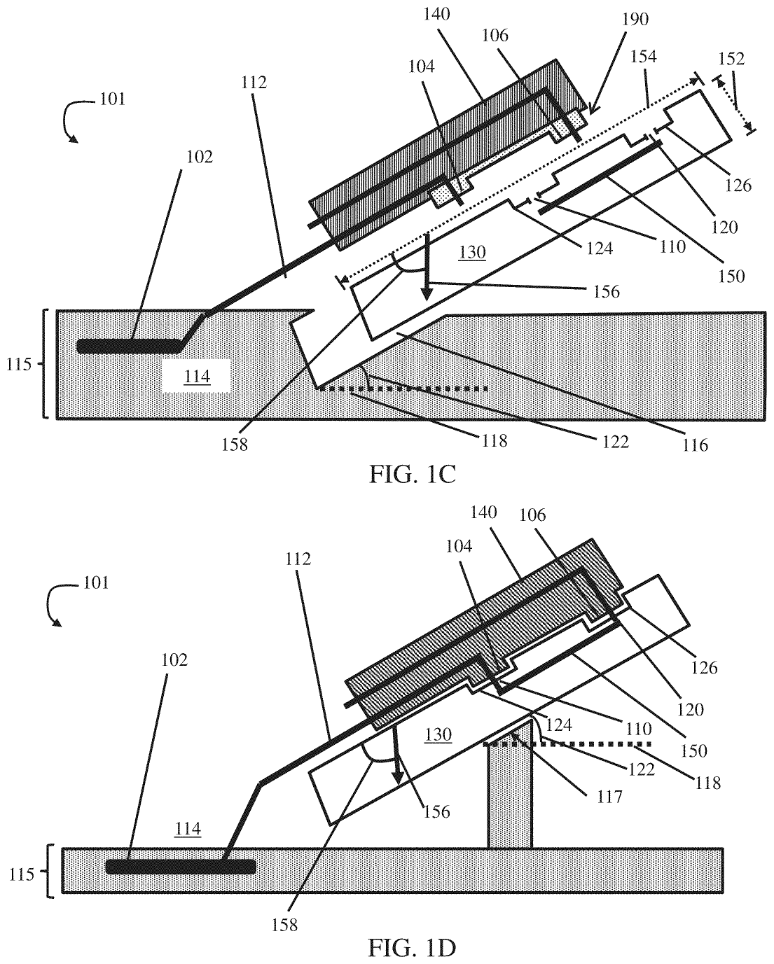

FIGS. 1A and 1B present an exemplary embodiment of fluidic device 101 in different configurations, according to certain embodiments. In FIGS. 1A-1B, fluidic device 101 comprises channel 102, located on cartridge 115 of fluidic device 101. As illustrated in FIGS. 1A-1B, in some embodiments, channel 102 of fluidic device 101 is fluidically connected to first alignment feature 104, disposed on discrete fluidic device portion 140. As illustrated in FIGS. 1A-1B, fluidic device 101 may also comprise second alignment feature 106. In some embodiments, such as the embodiment of fluidic device 101 in the configuration of FIG. 1A, second alignment feature 106 is not fluidically connected to channel 102. However, as illustrated in FIG. 1B, in some embodiments, second alignment feature 106 is fluidically connected to channel 102, e.g., via a fluidic connection to first alignment feature 104 created by fluidic pathway within fluidic receptacle 130, which has been received by fluidic device 101 in the illustrated configuration. In contrast, as illustrated in FIG. 1A, fluidic device 101 has not received fluidic device 130.

In some embodiments, the fluidic device comprises one or more discrete fluidic device portions. For example, in addition to a cartridge, the fluidic device may comprise a discrete fluidic device portion that can be fluidically connected to the cartridge. The discrete fluidic device portion may not be integrally formed with the cartridge. For example, in FIGS. 1A and 1B, fluidic device 101 comprises discrete fluidic device portion 140, fluidically connected to cartridge 115 via fluidic connection 112 (e.g., a conduit such as an interior of a tube) but not integrally formed with cartridge 115. In some embodiments, fluidic device portion 140 is connected to the cartridge mechanically, e.g., using fasteners. In some embodiments, alignment features of the fluidic device are located on the discrete fluidic device portion. For example, in FIGS. 1A-1B, alignment features 104 and 106 of fluidic device 101 are located on fluidic device portion 140. In some embodiments, this advantageously allows the discrete fluidic device portion to be manipulated such that it can more easily be coupled to the fluidic receptacle. The discrete fluidic device portion may have any of a variety of suitable geometries. For example, the fluidic device portion may be designed to interlock with a fluidic receptacle, as described in greater detail below.

In some embodiments, a first fluidic device portion is mechanically and/or fluidically coupled to a second fluidic device portion. For example, FIG. 1C presents an exemplary embodiment of a fluidic device 101, analogous to the fluidic device of FIGS. 1A-1B, where first fluidic device portion 140 is both mechanically and fluidically coupled to second fluidic device portion 190. In some embodiments, fluidic device 101 comprises first alignment feature 104, disposed on second fluidic device portion 190. In some embodiments, the first fluidic device portion and/or the second fluidic device portion are discrete fluidic device portions. Discrete fluidic device portions are not integrally connected to at least one component of the fluidic device (e.g., a cartridge), or to one another. For example, in FIG. 1C, fluidic device portions 140 and 190 are discrete fluidic device portions because they are neither integrally connected to each other nor are they integrally connected to solid substrate 114 of fluidic device 101. As illustrated in FIG. 1C, fluidic device 101 may also comprise second alignment feature 106, also disposed on second fluidic device portion 104.

Fluidic device portions (e.g., the first fluidic device portion and the second fluidic device portion) may be mechanically coupled to one another. For instance, the first fluidic device portion and the second fluidic device portion may be attached such that the mechanical coupling between the first fluidic device portion and the second fluidic device portion maintains the second fluidic device portion in a fixed position relative to the first fluidic device portion. The mechanical coupling between the first fluidic device portion and the second fluidic device portion may, in some cases, be accomplished using connection features of the first fluidic device portion and/or the second fluidic device portion. In some embodiments, a connection feature of the first fluidic device portion is configured to mechanically couple to a connection feature of a second fluidic device portion. The connection features may take any of a variety of appropriate forms and may couple by any of a variety of appropriate method. Non-limiting examples of connection features include pegs, protrusions, threaded cylinders, holes, recesses, and threaded holes. Connection features of the first fluidic device portion may couple to connection features of the second fluidic device portion by having a geometry complementary to a geometry of the connection features of the second fluidic device portion. For example, pegs, protrusions, threaded cylinders, holes, recesses, and/or threaded holes of the first fluidic device portion may have a geometry complementary to holes, recesses, threaded holes, pegs, protrusions and/or threaded cylinders of the second device portion, respectively. A connection feature of the first fluidic device portions and the second fluidic device portion can couple via, for example, snap fits, latches, clamps, mechanically interlocking features, interference fits, threaded connections, slot and tab connections, or combinations thereof. Although this paragraph refers to connections between a first fluidic device portion and a second fluidic device portion, it should be understood that connection features may generally be used to connect any fluidic device portion to any other fluidic device portion, depending on the embodiment, and the disclosure is not limited in this respect.

Fluidic device portions (e.g., the first fluidic device portion and the second fluidic device portion) may be fluidically coupled to one another. For instance, the first fluidic device portion and the second fluidic device portion may be fluidically connected, e.g., such that fluid can contact both the first fluidic device portion and the second fluidic device portion along a fluidic pathway to an alignment feature of the fluidic device. However, in some embodiments, fluidic device portions may not be fluidically connected. For instance, in some embodiments, a second fluidic device portion may extend through a first fluidic device portion, such that transmitted fluid does not contact the first fluidic device portion. The disclosure is not so limited.

In some embodiments, including both a first fluidic device portion and a second fluidic device portion can improve a fluidic connection between the fluidic device and a fluidic receptacle. For example, in some embodiments, the first fluidic device portion comprises a first material and the second fluidic device portion comprises a second material. In some embodiments, the second material is different than the first material. In some embodiments, the first material comprises a relatively rigid (e.g., a hard polymer) material. Exemplary relatively rigid materials may include glass, silicon, metal, or a relatively rigid polymer, such as a rigid silicone (e.g., PDMS), a polycarbonate, a polyolefin (e.g., polyethylene, polypropylene, a poly (cyclo olefin)), an acrylic (e.g., PMMA), polyoxymethylene, or combinations, mixtures, and/or copolymers thereof. In some embodiments, the second material may comprise a relatively flexible material (e.g., an elastomer, such as those commercialized by Precision Polymer Engineering Ltd., United Kingdom, or such as those commercialized by VersaFlex Inc., Kansas

City, Kansas). Non limiting examples of relatively flexible materials include silicones (e.g., PDMS), rubbers (e.g., polyisoprene, neoprene, ethylene propylene diene monomer rubber, nitrile), fluorinated elastomers (e.g., poly(tetrafluoro ethylene propylene), fluorosilicones, poly(tetrafluoro ethylene)), thermoplastic elastomers (e.g. styrenic block copolymers (TPE-S), polyolefin blends (TPE-O), elastomeric alloys, thermoplastic polyurethanes (TPE-U), thermoplastic copolyesters (TPE-E) and thermoplastic polyamides (TPE-A)), or combinations, mixtures, or copolymers thereof,

In some embodiments, the relatively rigid material has an average elastic modulus (a value of the elastic modulus averaged over its entire domain of elastic strain) of greater than or equal to 50 MPa, greater than or equal to 100 MPa, greater than or equal to 500 MPa, greater than or equal to 1 GPa, greater than or equal to 5 GPa, greater than or equal to 10 GPa, or greater. In some embodiments, the relatively flexible material has an average elastic modulus of less than or equal to 20 MPa, less than or equal to 10 MPa, less than or equal to 5 MPa, less than or equal to 2 MPa, less than or equal to 1 MPa, or less. Combinations of these ranges are possible. For example, in some embodiments, the relatively rigid material has an average elastic modulus of greater than or equal to 10 GPa and the relatively flexible material has an average elastic modulus of less than or equal to 50 MPa.

In some embodiments, a ratio between the average elastic modulus of the relatively rigid material and the average elastic modulus of the relatively flexible material is greater than or equal to 2, greater than or equal to 10, greater than or equal to 100, greater than or equal to 1,000, greater than or equal to 10,000, or greater. In some embodiments, the ratio between the average elastic modulus of the relatively rigid material and the average elastic modulus of the relatively flexible material is less than or equal to 1,000,000, less than or equal to 100,000, less than or equal to 10,000, or less. Combinations of these ranges are possible. For example, in some embodiments, the ratio between the average elastic modulus of the relatively rigid material and the average elastic modulus of the relatively flexible material may be greater than or equal to 2 and less than or equal to 1,000,000.

In some embodiments, the second fluidic device portion is configured to at least partially seal (e.g., partially seal or completely seal) a fluidic connection formed by coupling an alignment feature disposed on the second fluidic device portion with an alignment feature of a fluidic receptacle. For example, in some embodiments, the second fluidic device portion forms a seal (e.g., a pressure seal) around the fluidic connection that reduces or prevents leaking from the fluidic connection, relative to embodiments using only a single fluidic device portion, but allows fluidic transfer between the coupled alignment features. This may advantageously reduce or eliminate the need for an intervening part (e.g., an additional gasket such as an O-ring) between the alignment features. Meanwhile, the relatively rigid first fluidic device portion may be better suited to maintaining its shape when acted upon by external forces. This may help to maintain the configuration of the fluidic device, by reducing or preventing unwanted deformation (e.g., when the fluidic device is under external stress from a user or other components of a system).

The second fluidic device portion may, in some cases, be made separately from the first fluidic device portion. For example, the second fluidic device portion may be cast or molded into an appropriate geometry. In some embodiments, the second fluidic device portion may be formed directly on the first fluidic device portion (e.g., the second fluidic device portion may be molded onto the first fluidic device portion).

A fluidic receptacle, according to certain embodiments, may be used to house and transport an integrated device (e.g., an integrated device for analysis of a target molecule). For example, the fluidic receptacle may contain the integrated device (e.g., within an interior of the fluidic receptacle). In some embodiments, transporting fluid to a fluidic receptacle is necessary for the loading of the integrated device. The fluidic receptacle, once loaded, may be used to transport the loaded integrated device (e.g., to transport the integrated device to a detection module, as described in further detail below).

The fluidic receptacle comprises alignment features, according to some embodiments. For example, alignment features may be located on the fluidic receptacle. In some embodiments where both the fluidic device and the fluidic receptacle comprise alignment features, each can comprise multiple alignment features. For example, in some embodiments, the fluidic device comprises a first alignment feature and a second alignment feature, and the fluidic receptacle comprises a third alignment feature and, in some instances, a fourth alignment feature. According to some embodiments, the alignment features of the fluidic receptacle couple to alignment features of the fluidic device. For example, the alignment features of the fluidic receptacle may couple to the alignment features of the fluidic device mechanically, fluidically, or both.

For example, in FIGS. 1A-1B, fluidic receptacle 130 comprises third alignment feature 124, configured to couple to first alignment feature 104 of fluidic device 101. Furthermore, in FIGS. 1A-1B, fluidic receptacle 130 comprises fourth alignment feature 126, configured to couple to second alignment feature 106 of fluidic device 101.

According to some embodiments, the fluidic receptacle is coupleable to a detection module (e.g., sequencing module) downstream of the fluidic devices (with respect to a workflow) for transferring fluid described herein. For example, the fluidic receptacle can be removed from the fluidic device and coupled to the detection module, according to certain embodiments. Examples of workflows where a fluidic receptacle is loaded and coupled to a detection module, according to certain embodiments, are provided below.

Some aspects are directed towards methods of transferring fluids. According to some embodiments, a fluidic receptacle is coupled to two or more alignment features (e.g., alignment features of a fluidic device). In some embodiments, a fluidic device is aligned by coupling two or more alignment features. For example, the fluidic device may be aligned with a fluidic receptacle by coupling two or more alignment features of the fluidic device to the fluidic receptacle. According to some embodiments, a fluid is transferred via at least one of the alignment features. For example, according to some embodiments, the fluid is transferred to the fluidic receptacle. Further, according to some embodiments, the fluidic receptacle is decoupled from the alignment features to which it was originally coupled.

The fluidic receptacle may have any of a variety of appropriate geometries. For example, the fluidic receptacle may have a substantially rectangular lateral profile, according to certain embodiments. In some embodiments, the fluidic receptacle has a narrow transverse dimension, when compared to a minimum lateral dimension of the fluidic receptacle. In some embodiments, the fluidic receptacle may be configured to house a substrate (e.g., an integrated device, a chip). According to some embodiments, the fluidic receptacle is a flow cell (e.g., a cell comprising an interior, an inlet, and an outlet, configured to receive a fluid through an inlet and transmit a fluid through an outlet). For example, the fluidic receptacle may be a flow cell comprising an integrated device. In some embodiments, the fluidic receptacle has an interior (e.g., an interior chamber). In some embodiments, the interior of the device is capable of being at least partially or completely filled with a fluid. The interior of the fluidic device has a volume, in some embodiments. In some embodiments, the fluidic receptacle has more than one interior. For example, the fluidic receptacle may comprise 1, 2, 3, 4, 5, or more interiors. According to some embodiments, at least some of these interiors are fluidically connected to one another. In some embodiments, these interiors are divided from one another, such that fluid cannot flow directly from one interior to another. The inclusion of multiple interiors of the fluidic receptacle is advantageous, according to some embodiments, because it can allow multiple integrated devices or integrated device portions to be filled simultaneously. The simultaneous filling of multiple integrated device or integrated device portions may allow greater uniformity of fluid flow, and/or may allow the analysis of multiple samples that are simultaneously allowed to interact with separate integrated devices or integrated device portions.

Each interior of the fluidic receptacle may comprise an inlet and an outlet. According to some embodiments, the inlet and/or the outlet can be connected to the fluidic device (e.g., via one or more alignment features). In some embodiments, fluid (e.g., a fluid sample comprising a biomolecules such as a peptide or polynucleotide) is flowed through one or more interiors of the fluidic receptacle. Flowing fluid through an interior can, in some embodiments, at least partially or completely fill the interior of the fluidic receptacle.

For example, in FIGS. 1A-1B, fluidic receptacle 130 comprises inlet 110, outlet 120, and interior 150, in accordance with some embodiments. In FIG. 1B, fluid is flowed through interior 150 (e.g., to fill interior 150) via the fluidic connections formed by the coupling of alignment features 104 and 106 of fluidic device 101 with alignment features 124 and 126 of fluidic receptacle 130, respectively, in some embodiments.

In some embodiments where the fluidic receptacle comprises more than one interior, the interiors of the fluidic device can be filled with the same fluid. For example, in some embodiments, a first interior of a fluidic receptacle comprising more than one interior is filled with a first fluid (e.g., fluid from a first peptide sample), while a second interior of the fluidic receptacle comprising more than one interior is also filled with the first fluid (e.g., fluid from the first peptide sample). In some embodiments, where the fluidic receptacle comprises more than one interior, the interiors of the fluidic device can be filled with different fluids. For example, in some embodiments, a first interior of a fluidic receptacle comprising more than one interior is filled with a first fluid (e.g., fluid from a first peptide sample), while a second interior of the fluidic receptacle comprising more than one interior is filled with a second fluid (e.g., fluid from a second peptide sample).

According to certain embodiments, a fluidic receptacle comprises a transverse dimension. For example, in FIG. 1A, fluidic receptacle 130 comprises transverse dimension 152. In some embodiments, a fluidic receptacle comprises a lateral dimension. For example, in FIG. 1B, fluidic receptacle 130 comprises lateral dimension 154. In some embodiments, the fluidic receptacle is substantially wider along its lateral dimensions than along a transverse dimension perpendicular to its lateral dimensions. For example, according to some embodiments, a ratio between a minimum lateral dimension of the fluidic receptacle and a maximum transverse dimension of the fluidic receptacle is greater than or equal to 5, greater than or equal to 10, greater than or equal to 15, greater than or equal to 20, greater than or equal to 50, and/or up to 100, up to 1000, or greater.

The fluidic receptacle may have any of a variety of convenient lateral profiles. For example, according to some embodiments, the fluidic receptacle has a lateral profile that is rectangular. According to some embodiments, the lateral profile has an aspect ratio. In some embodiments, the lateral profile has an aspect ratio of greater than or equal to 0.1, greater than or equal to 0.2, greater than or equal to 0.4, greater than or equal to 0.5, greater than or equal to 0.66, greater than or equal to 1, greater than or equal to 1.5, greater than or equal to 2, greater than or equal to 2.5, greater than or equal to 5, or greater. In some embodiments, the lateral profile has an aspect ratio of less than or equal to 10, less than or equal to 5, less than or equal to 2.5, less than or equal to 2, less than or equal to 1.5, less than or equal to 1, less than or equal to 0.66, less than or equal to 0.5, less than or equal to 0.4, less than or equal to 0.2, or less. Combinations of these ranges are possible. For example, in some embodiments, lateral profile has an aspect ratio of greater than or equal to 0.1 and less than or equal to 10.

In some embodiments, the fluidic device is configured to receive the fluidic receptacle in an alignment (e.g., an alignment with respect to the fluidic device). In some embodiments, the fluidic device is configured to receive the fluidic receptacle via a coupling between alignment features (e.g., a coupling between alignment features of the fluidic device and alignment features of the fluidic receptacle). Alignment features may couple by having complementary shapes, by being able to interlock, by being configured to connect to an intervening part (e.g., a peg), or by any other appropriate mechanism. In some embodiments, a first coupling is formed between the first alignment feature of the fluidic device and the third alignment feature of the fluidic receptacle, as described above. In some embodiments, a second coupling is formed between the second alignment feature of the fluidic device and the fourth alignment feature of the fluidic receptacle, as described above. As an example, in FIG. 1B, first alignment feature 104 and third alignment feature 124 are coupled using their complementary shapes (a protrusion and a recess), as are second alignment feature 106 and fourth alignment feature 126.

An alignment features described herein may comprise any structural component that can facilitate alignment of the fluidic device with the fluidic receptacle. For example, in FIGS. 1A-1B, first alignment feature 104 and second alignment feature 106 of fluidic device 101 comprise protrusions, while third alignment feature 124 and fourth alignment feature 126 comprises recesses configured to receive the protrusions.

In some embodiments, alignment features are coupled directly. However, direct coupling between alignment features is not required, and alignment features may instead be coupled, using 1, 2, 3, 4, 5, or more intervening parts. For example, both the fluidic device and the fluidic receptacle comprise holes, in some embodiments, which may each be coupled to an intervening part such as a peg. According to certain embodiments, the intervening part are used for sealing a fluidic connection between alignment features. For example, the intervening part comprises a gasket (e.g., an O-ring), according to certain embodiments. In some embodiments, the intervening component is a molded layer (e.g., a layer molded to conform to a configuration of an alignment feature).

According to some embodiments, alignment features of the fluidic device are of the same type as alignment features of the fluidic receptacle. For example, in some embodiments the alignment features of the fluidic device and the alignment features of the fluidic receptacle comprise holes, as described in the preceding paragraph. In some embodiments, alignment features of the fluidic device are complementary to alignment features of the fluidic receptacle. According to some embodiments, complementary alignment features interlock. For example, in FIGS. 1A-1B, alignment features 104 and 106 of fluidic device 101, which comprises protrusions, are complementary to alignment features 124 and 126 of fluidic receptacle 130, which comprise recesses configured to receive those protrusions directly.

Although some examples of alignment features, such as recesses and protrusions, have been provided, any of a variety of suitable alignment feature geometries may be used. For example, a protrusion may be a rectangular protrusion, a rounded protrusion, a hexagonal protrusion, a hollow screw, or a cylindrical protrusion. A recess may be a rectangular recess, a rounded recess, a hexagonal recess, a smooth cylindrical recess, or a threaded cylindrical recess.

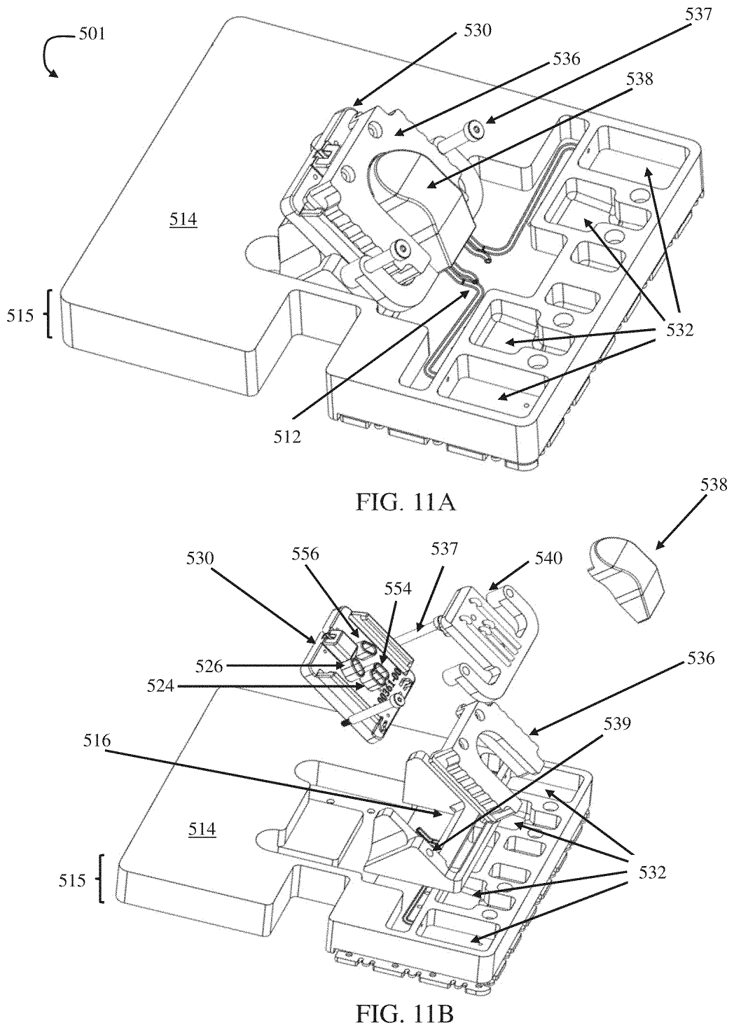

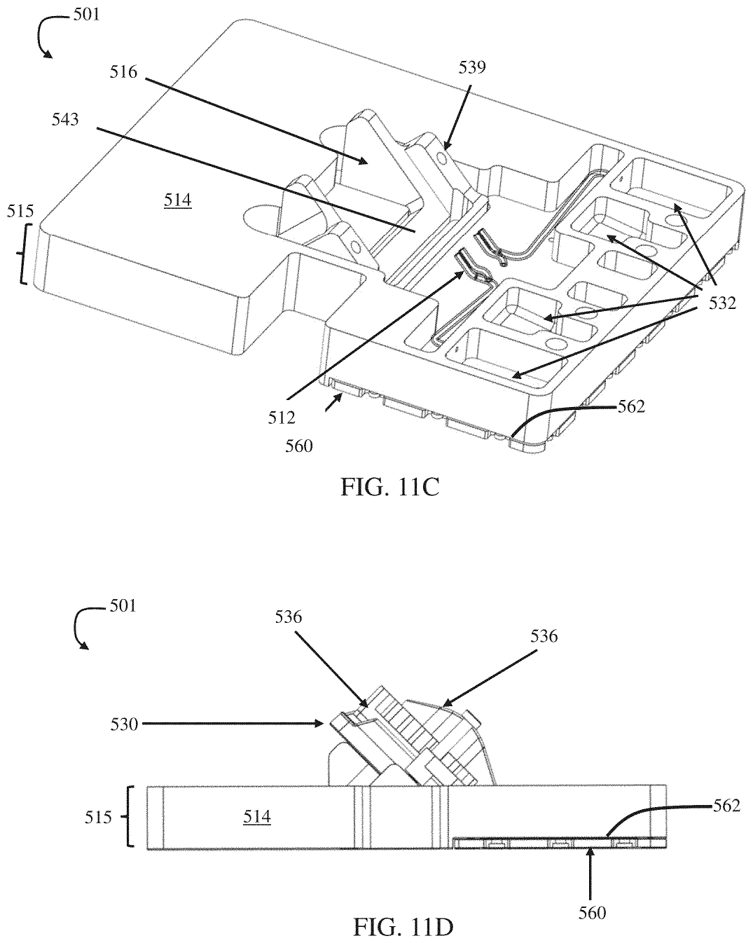

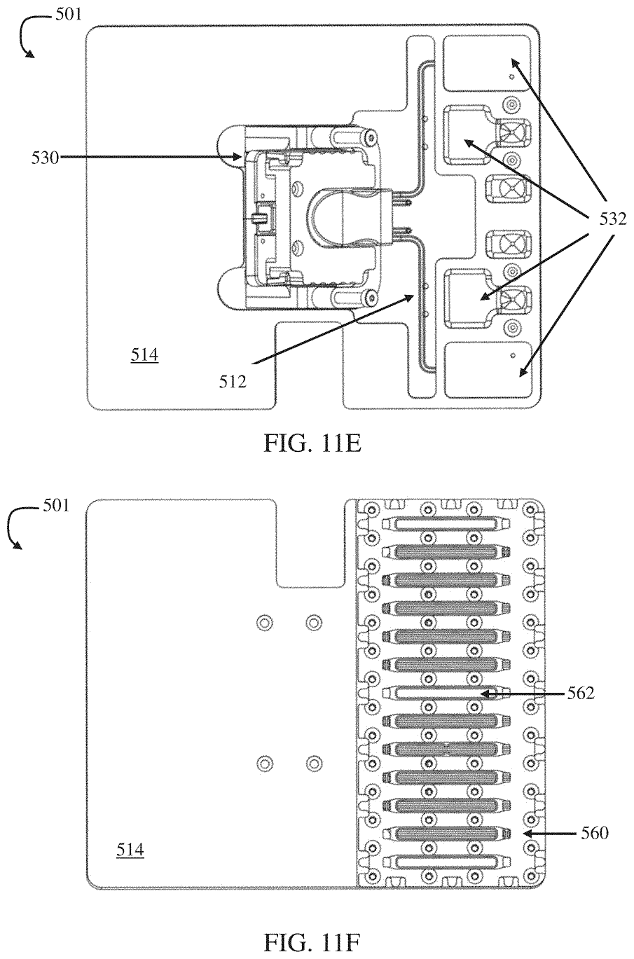

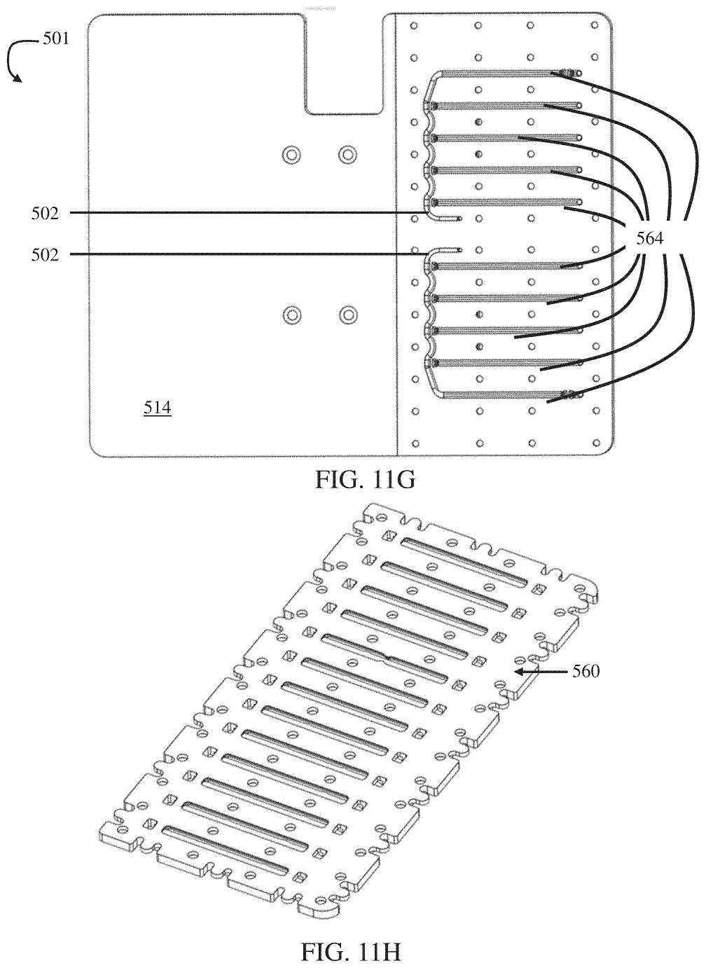

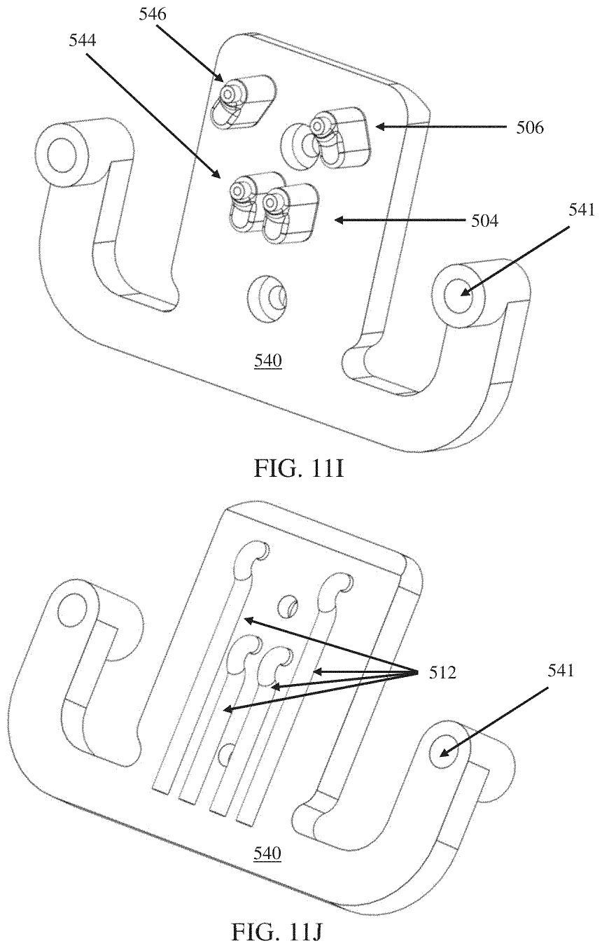

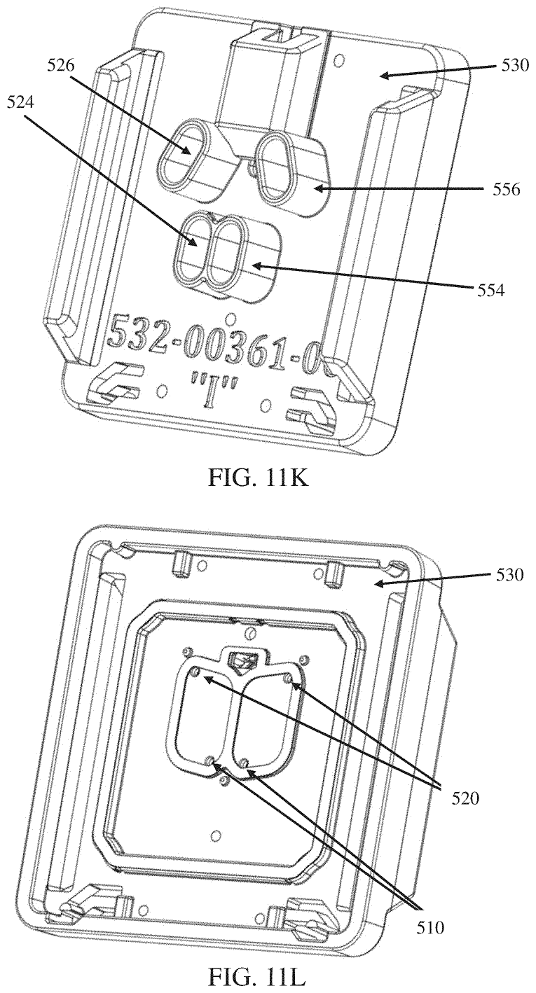

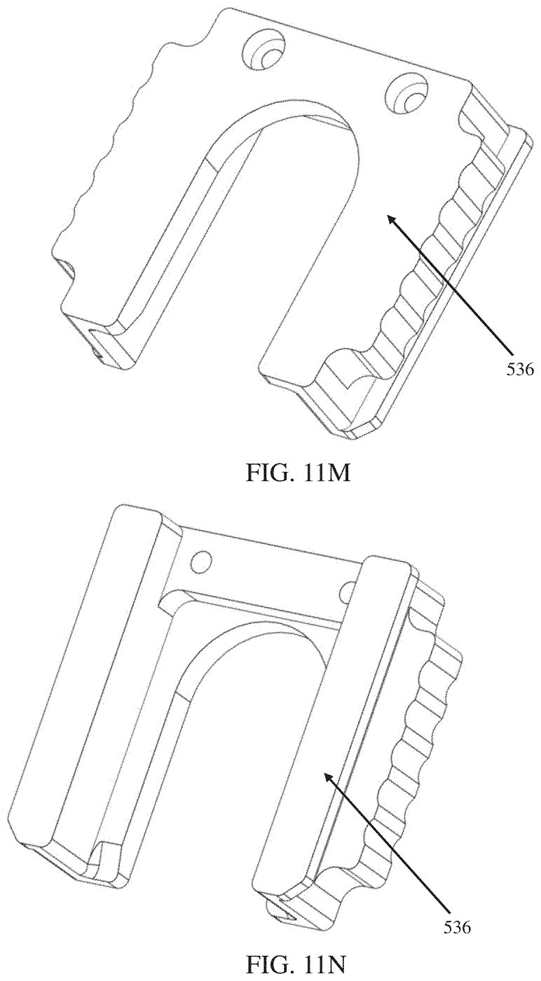

According to some embodiments, the fluidic device and the fluidic receptacle can be fluidically connected through one or more alignment features. For example, according to some embodiments, a channel of the fluidic device is fluidically connected, or is configured to be fluidically connected, to the fluidic receptacle through the first alignment feature. In some embodiments, a channel of the fluidic device is fluidically connected, or is configured to be fluidically connected, to the fluidic receptacle through the second alignment feature. In some embodiments, a channel of the fluidic device is fluidically connected, or is configured to be fluidically connected, to the fluidic receptacle through the third alignment feature, located on the fluidic receptacle. According to some embodiments, a channel of the fluidic device is fluidically connected, or is configured to be fluidically connected, to the fluidic receptacle through a fourth alignment feature, located on the fluidic receptacle. According to some embodiments, a channel of the fluidic device is fluidically connected, or is configured to be fluidically connected, to the fluidic receptacle through a fifth alignment feature, located on the fluidic device. In some embodiments, a channel of the fluidic device is fluidically connected, or is configured to be fluidically connected, to the fluidic receptacle through a sixth alignment feature, located on the fluidic receptacle. In some embodiments, a channel of the fluidic device is fluidically connected, or is configured to be fluidically connected, to the fluidic receptacle through the seventh alignment feature, located on the fluidic device. In some embodiments, a channel of the fluidic device is fluidically connected, or is configured to be fluidically connected, to the fluidic receptacle through an eighth alignment feature, located on the fluidic receptacle. Examples of fifth, sixth, seventh, and eighth alignment features are provided below, with reference to FIGS. 11A-11N.

Forming a fluidic connection through an alignment feature present a number of advantages, in some embodiments. For example, a fluidic connection through a coupling between an alignment feature of a fluidic device and an alignment feature of the fluidic receptacle may be mechanically supported by the coupling. Furthermore, fluidically connecting the fluidic device and the fluidic receptacle via the coupling may reduce the need for intervening components such as O-rings, advantageously reducing a dead volume of the fluidic connection and thereby eliminating waste of samples and/or reagents.

According to some embodiments, the fluidic device comprises at least 1, at least 2, at least 3, at least 4, at least 5, at least 7, at least 10, or more alignment features. The alignment features of the fluidic device may be the same or different. In some embodiments, the fluidic receptacle comprises at least 1, at least 2, at least 3, at least 4, at least 5, at least 7, at least 10 or more alignment features. The alignment features of the fluidic receptacle may be the same or different. In some embodiments, the fluidic device is configured to receive (e.g., hold) the fluidic receptacle in an alignment with respect to the fluidic device. In some embodiments, the fluidic device and the fluidic receptacle are coupled by at least 1, at least 2, at least 3, at least 4, at least 5, at least 7, at least 10, or more couplings between alignment features of the fluidic receptacle and alignment features of the fluidic device. For example, according to some embodiments, the fluidic device is configured to be coupled to the fluidic receptacle via a coupling between a fifth alignment feature, located on the fluidic device, and a sixth alignment feature, located on the fluidic receptacle. In some embodiments, the fluidic device is configured to be coupled to the fluidic receptacle via a coupling between a seventh alignment feature, located on the fluidic device, and an eighth alignment feature, located on the fluidic receptacle.

According to some embodiments, the formation of the fluidic connection between alignment features results from the coupling between the alignment features. For example, according to some embodiments, coupling an alignment feature of the fluidic device with an alignment feature of the fluidic receptacle result in the formation of a fluidic connection between a tube of the fluidic device and an inlet or outlet of the fluidic receptacle. However, in some embodiments, the fluidic connection is formed separately. For example, according to some embodiments, the tube is connected to the inlet or outlet prior to the coupling of the alignment features.

In some embodiments, the fluidic device comprises a mounting element. Exemplary mounting elements may comprise recesses, raised edges, friction-inducing features, clamps, clips, grips, rails, and/or any of a variety of other features capable of receiving the fluidic receptacle into the mounting element. For example, the mounting element may be a recess, a raised edge, a friction-inducing feature, a clamp, a clip, or a grip. In some embodiments, a mounting element comprises one or more mounting features. Mounting features may be configured help affix the fluidic receptacle to the fluidic device.

Mounting features may be configured to at least partially align a fluidic device portion with a fluidic receptacle. Any of a variety of appropriate mounting features of the mounting element may be used, including screws, bolts, and/or posts that extend from the mounting element. Additional examples of mounting features include holes or recesses configured to receive secondary mounting features such as screws, bolts, or posts. For example, mounting features may be configured to receive secondary mounting features such as bolts, screws, posts, or any of a variety of other secondary mounting features extending directly from a fluidic device portion. Additionally or alternatively, mounting features may be configured to receive secondary mounting features such as independent bolts, screws, posts, or any of a variety of other features. Secondary mounting features may be mechanically coupled to one or more discrete fluidic device portions. Secondary mounting features may be used to at least partially (e.g., partially or completely) align the fluidic device portion. For example, the bolts, screws, posts, or other secondary mounting features may pass through holes of the fluidic device portion. Partially aligning the fluidic device portion using the mounting features may advantageously simplify alignment of the alignment features of the fluidic device portion with alignment features of the fluidic receptacle. For instance, the mounting features may reduce the range of motion of the fluidic device portion, relative to the fluidic receptacle. Examples of mounting features are described below, with reference to specific embodiments of the fluidic device.

In some embodiments, the mounting element comprises a surface angled with respect to a lateral dimension of the device. For example, in some embodiments, the fluidic device comprises a recess. The recess comprises, in some embodiments, a surface angled with respect to a lateral dimension of the fluidic device. For example, in FIGS. 1A-1B, recess 116 comprises a bottom surface angled at angle 122 with respect to lateral dimension 118 of fluidic device 101, according to certain embodiments. FIG. 1D presents an embodiment similar to the embodiment of FIG. 1B, but where a mounting element 117 is not a recess in fluidic device 101. A variety of non-limiting types of mounting elements are described in greater detail below.

In some embodiments, the mounting element is within a solid substrate of the fluidic device. For example, in some embodiments, the recess is within a solid substrate of the fluidic device. The solid substrate may be completely solid, or the solid substrate may have channels, reservoirs, inlets, outlets, holes, or other non-uniformities. For example, recess 116 is in solid substrate 114, in accordance with some embodiments.

In some embodiments, the mounting element is in a raised portion of a fluidic device. For example, the fluidic device may comprise a raised portion comprising a mounting element, configured to receive the fluidic receptacle in a raised position, relative to at least a portion (e.g., a portion or all) of the solid substrate. For example, exemplary mounting element 117 shown in FIG. 1D is shown to be in a raised portion of a fluidic device relative to solid substrate 114. In some embodiments the mounting element is integrally formed in a cartridge of a fluidic device. For example, in FIGS. 1A-1C, mounting element 116 is integrally formed in cartridge 115 of fluidic device 101. However, the mounting element may also be a discrete object that is mechanically coupled to the fluidic device (e.g., using an adhesive, a clip, an interlocking feature, screws, nails, clips, or any of a variety of other appropriate methods of mechanical coupling). For example, in FIG. 1D, mounting element 117 is a discrete object, coupled to cartridge 115.

The mounting element (e.g., recess, raised edge, friction-inducing feature) may be configured to receive the fluidic receptacle. In some embodiments, the recess is configured to receive the fluidic receptacle (e.g., into the recess, against the raised edge, onto the friction-inducing feature). The mounting element may be configured, such that the fluidic receptacle rests in or against the mounting element. In some embodiments, the fluidic receptacle rests in the mounting element. For example, the recess may be configured such that the fluidic receptacle rests in the recess (e.g., on a ramp of the solid substrate), or so that the fluidic receptacle is held in place by the recess (e.g., within a slot of the solid substrate. In some embodiments, the fluidic device is configured such that the fluidic receptacle rests against the mounting element. For example, the fluidic receptacle may be configured to rest against a raised edge or a friction-inducing feature, in some embodiments. For example, in FIG. 1D, fluidic receptacle 130 is configured to rest against a friction-inducing feature of mounting element 117 (in this case, a friction inducing surface of mounting element 117). Friction-inducing features may have a high coefficient of static friction that helps hold the fluidic device in position, in some embodiments. Exemplary friction-inducing features may include friction-inducing surfaces, such as treads or rough surfaces, in some embodiments. According to some embodiments, the fluidic receptacle is configured to rest against a surface (e.g., an angled surface) of a mounting element. For instance, in FIG. 1D, the fluidic receptacle is configured to rest against the friction-inducing surface of mounting element 117, which is angled with respect to a lateral dimension of fluidic device 101.

In at least one configuration (e.g., at least one alignment or arrangement), the received fluidic receptacle may be fluidically connected to the channel of the fluidic device when received into the mounting element. For example, in at least one configuration (e.g., at least one alignment or arrangement), the received fluidic receptacle is fluidically connected to the channel of the fluidic device when received into the recess, according to some embodiments. However, in some configurations, the fluidic receptacle is in or against the mounting element but not fluidically connected to the channel. For example, the fluidic receptacle may be in the recess but not fluidically connected to the channel of the fluidic device. The angle of the surface may facilitate an orientation of the received fluidic receptacle that advantageously facilitates the separation of gases (e.g., air bubbles) from liquids in the fluid, while maintaining an ergonomic orientation of the received fluidic receptacle. Thus, the appropriate orientation of the fluidic receptacle may advantageously improve sample loading, while allowing the fluidic device to occupy a smaller space, and while, in some instances, providing a convenient arrangement for a user of the fluidic system to insert and/or remove the fluidic receptacle with respect to the fluidic device. In some embodiments, the recess configured to receive the fluidic receptacle (e.g., recess 116) has a volume that is at least 5%, at least 10%, at least 25%, at least 50%, at least 75% of more of the overall volume of the solid substrate of the fluidic device.

According to some embodiments, an angle between the surface of the mounting element (e.g., the recess, the raised edge) and the lateral dimension of the fluidic device is greater than 0°, greater than or equal to 5°, greater than or equal to 10°, greater than or equal to 15°, greater than or equal to 20°, greater than or equal to 25°, greater than or equal to 30°, greater than or equal to 35°, greater than or equal to 40°, greater than or equal to 45°, greater than or equal to 60°, greater than or equal to 75°, or greater. According to some embodiments, the angle between the surface and the lateral dimension of the fluidic device is less than 90°, less than or equal to 85°, less than or equal to 80°, less than or equal to 75°, less than or equal to 70°, less than or equal to 65°, less than or equal to 60°, less than or equal to 50°, less than or equal to 45°, less than or equal to 30°, or less. Combinations of these ranges are possible. For example, according to some embodiments the angle between the surface and the lateral dimension of the fluidic device is greater than 0° and less than 90°. As a specific example, according to some embodiments the angle between the surface and the lateral dimension of the fluidic device is greater than or equal to 10° and less than or equal to 80°. As another specific example, according to some embodiments, the angle between the surface and the lateral dimension of the fluidic device is greater than or equal to 25° and less than or equal to 65°. One example of such an angle is angle 122, illustrated in FIG. 1B, which is between lateral dimension 118 of fluidic device 101 and a surface of recess 116, according to some embodiments. Another example of such an angle is angle 122, illustrated in FIG. 1D, which is between lateral dimension 118 of fluidic device 101 (vertically off-set from the device for visual clarity) and a surface of mounting element 117, according to some embodiments.

As noted above, in some embodiments the mounting element includes a recess (e.g., within a solid substrate of the fluidic device). According to some embodiments, an angle between the surface of the recess and the lateral dimension of the fluidic device is greater than 0°, greater than or equal to 5°, greater than or equal to 10°, greater than or equal to 15°, greater than or equal to 20°, greater than or equal to 25°, greater than or equal to 30°, greater than or equal to 35°, greater than or equal to 40°, greater than or equal to 45°, greater than or equal to 60°, greater than or equal to 75°, or greater. According to some embodiments, the angle between the surface and the lateral dimension of the fluidic device is less than 90°, less than or equal to 85°, less than or equal to 80°, less than or equal to 75°, less than or equal to 70°, less than or equal to 65°, less than or equal to 60°, less than or equal to 50°, less than or equal to 45°, less than or equal to 30°, or less. Combinations of these ranges are possible. For example, according to some embodiments the angle between the surface and the lateral dimension of the fluidic device is greater than 0° and less than 90°. As a specific example, according to some embodiments the angle between the surface and the lateral dimension of the fluidic device is greater than or equal to 10° and less than or equal to 80°. As another specific example, according to some embodiments, the angle between the surface and the lateral dimension of the fluidic device is greater than or equal to 25° and less than or equal to 65°. An example of such an angle is angle 122, illustrated in FIG. 1B, which is between lateral dimension 118 of fluidic device 101 and a surface of recess 116, according to some embodiments.

In some embodiments, during the transfer of fluid from the fluidic device to the fluidic receptacle, a lateral dimension of the fluidic receptacle is at an angle with respect to a direction of earth's gravity. For example, in FIG. 1B, during the transfer of fluid to fluidic receptacle 130, angle 158 is shown between direction of earth's gravity 156 and a lateral dimension of fluidic receptacle 130. According to some embodiments, angle between a lateral dimension of the fluidic receptacle and a direction of earth's gravity is greater than 0°, greater than or equal to 5°, greater than or equal to 10°, greater than or equal to 15°, greater than or equal to 20°, greater than or equal to 25°, greater than or equal to 30°, greater than or equal to 35°, greater than or equal to 40°, greater than or equal to 45°, greater than or equal to 60°, greater than or equal to 75°, or greater. According to some embodiments, the angle between a lateral dimension of the fluidic receptacle and the direction of earth's gravity is less than 90°, less than or equal to 85°, less than or equal to 80°, less than or equal to 75°, less than or equal to 70°, less than or equal to 65°, less than or equal to 60°, less than or equal to 50°, less than or equal to 45°, less than or equal to 30°, or less. Combinations of these ranges are possible. For example, according to some embodiments the angle between a lateral dimension of the fluidic receptacle and the direction of earth's gravity is greater than 0° and less than 90°. As a specific example, according to some embodiments the angle between the surface and the lateral dimension of the fluidic receptacle is greater than or equal to 10° and less than or equal to 80°. As another specific example, according to some embodiments, the angle between the surface and the lateral dimension of the fluidic receptacle is greater than or equal to 25° and less than or equal to 65°.

According to some embodiments, the fluidic receptacle comprises an integrated device (e.g., a device integrating a sensor and a sample well, configured to receive a sample). The integrated device may be configured to facilitate interrogation and/or detection of fluid inside the integrated device. For example, the integrated device may be configured to facilitate sequencing of molecules of interest such as biopolymers (e.g., peptides, nucleic acids) within the fluid. In some embodiments, the integrated device is within the fluidic receptacle (e.g., within an interior of the fluidic receptacle). In some embodiments, the fluidic receptacle contains or is configured to contain the integrated device. The integrated device may be a part of the fluidic receptacle (e.g., integrally formed with the fluidic receptacle).

The integrated device is removable from the fluidic receptacle, in some embodiments. This may advantageously allow the coupling of the integrated device with a sequencing module or a detection module configured to receive the integrated device without the remainder of the fluidic receptacle. In some embodiments, the integrated device is not removable from the fluidic receptacle. In some embodiments, the entire fluidic receptacle may be coupled to a detection module or a sequencing module configured to receive the entire fluidic receptacle. Referring to FIG. 1B, in an exemplary, non-limiting embodiment, an integrated device is contained within interior 150 of fluidic receptacle 130, while a fluid comprising a sample (e.g., a peptide) is flowed through interior 150. This may result in the loading of the integrated device with the fluid. Subsequently, in some embodiments, the loaded integrated device may be analyzed using a detection module or sequencing module, as described in more detail below.

The integrated device may include a plurality (e.g., an array) of pixels. In some embodiments, individual pixels include a sample well, configured to receive a sample. The pixels may further include at least one sensor (e.g., a photodetector). In some embodiments, the integrated device may include components that generate a radiation pattern based on emission energy emitted from a sample. Emission energy emitted by a sample may then be detected by one or more sensors within a pixel of the integrated device. In some embodiments one or more sensors are configured to detect timing characteristics associated with a sample's emission energy (e.g., fluorescence lifetime). In some embodiments, the sensors are configured to detect a spatial distribution of at least a portion of a radiation pattern.

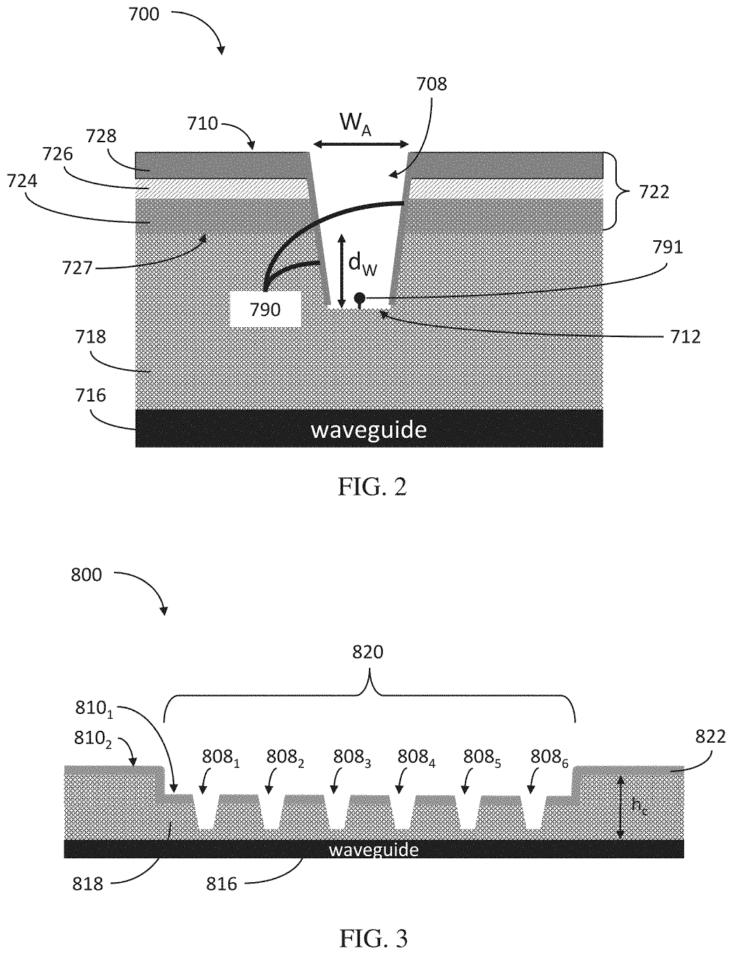

In some embodiments, a fluidic device is used to load a molecule of interest into a sample well by contacting a sample having the molecule of interest to a surface of an integrated device. For example, the fluidic device may be used to load fluid into a fluidic receptacle comprising the integrated device, such that the fluid is brought into contact with the surface of the fluidic device. In some embodiments, the integrated device comprises the sample well. For example, FIG. 2 is a cross-sectional view of integrated device 707 comprising sample well 708, according to some embodiments. A sample well 708 may comprise a small volume or region at surface 710 of integrated device 707, which is distal to bottom surface 712 of sample well 708. Sample well 708 may be configured to receive a sample comprising molecule of interest 791, which can be retained at bottom surface 712 of sample well 708. Bottom surface 712 of sample well 708 comprises one or more coupling groups that bind to molecule of interest 791, at least temporarily for a duration of time. Bottom surface 712 of sample well 708 may have one or more materials that provide selectivity for molecule of interest 791 to adhere to bottom surface 712 rather than the side walls 790 of sample well 708. In some embodiments, bottom surface 712 and side walls 790 of sample well 708 may be prepared (e.g., passivated, functionalized, etc.) using techniques described herein or methods known in the art.

In some embodiments, molecule of interest 791 is disposed within sample well 708 through a top aperture that is distal to bottom surface 712 of sample well 708. According to some embodiments, the molecule of interest is transferred to the sample well via a fluid loaded into a fluidic receptacle using the methods, systems, and articles described herein. The top aperture may be configured to reduce ambient light or stray light from illuminating molecule of interest 791 within sample well 708. The top aperture may have a width W_{A}, as measured at a surface 710 of integrated device 707, that is in the range of 50 nm and 300 nm, or any value or range of values within that range. Sample well 708 may have a depth d_{W} between bottom surface 712 and interface 727 between top cladding 718 and metal layer 722. Depth d_{W} may provide a suitable distance between a molecule of interest positioned at bottom surface 712 and metal layer 722. Depth d_{W} may impact the timing of photon emission events of a marker (e.g., lifetime) associated with molecule of interest 791. Accordingly, depth d_{W} may allow for distinguishing among different markers in sample well 708 based on timing characteristics associated with the individual lifetimes of the different markers. In some embodiments, depth d_{W} of sample well 708 may impact the amount of excitation energy received. Depth d_{W} may be in the range of 50 nm to 350 nm, or any value or range of values within that range. In some embodiments, depth d_{W} is between 95 nm and 150 nm. In some embodiments, depth d_{W} is between 150 nm and 350 nm. In some embodiments, depth d_{W} is between 200 nm and 325 nm. In some embodiments, depth d_{W} is between 250 nm and 300 nm. In some embodiments, depth d_{W} is approximately 270 nm.

In some embodiments, the integrated device comprises a waveguide. For example, in various embodiments, sample well 708 may be arranged to receive excitation energy from waveguide 716. Waveguide 716 may be configured to provide an optical mode that evanescently decays from the waveguide. In some embodiments, the evanescent field of the mode may overlap, at least in part, with sample well 708. In this way, molecule of interest 791 within sample well 708 may receive excitation energy through the evanescent field of the optical mode.

Integrated device 707 may include metal layer 722 over top cladding 718. Metal layer 722 may act as a reflector for emission energy emitted by a sample in a sample well and may improve detection of emission energy by reflecting emission energy towards a sensor of the integrated device. Metal layer 722 may act to reduce the background signal due to photons that do not originate within the sample well. Metal layer 722 may comprise one or more sub-layers. Examples of suitable materials to be used as layers of a metal layer may include aluminum, copper, titanium, and titanium nitride. As shown in FIG. 2, metal layer 722 includes first sub-layer 724, second sub-layer 726, and third sub-layer 728. The thickness of the first sub-layer may be in the range of 30 nm to 165 nm, or any value or range of values within that range. The thickness of the second sub-layer may be in the range of 5 nm to 100 nm, or any value or range of values within that range. In some embodiments, the thickness of the second sub-layer may be approximately 10 nm. The third sub-layer may have a thickness in the range of 5 nm to 100 nm, or any value or range of values within that range. In some embodiments, the third sub-layer may have a thickness of approximately 30 nm.

Sample well 708 may have one or more side walls covered, at least partially, with a sidewall spacer on side walls 790. The composition of a sidewall spacer may be such that the side walls 790 are configured to enable a certain type of interaction with molecule of interest 791. In some embodiments, a sidewall spacer may have a composition configured to passivate the side walls of sample well 708 to reduce the amount of molecule of interest 791 that adheres to the side walls 790. By coating only the side walls of the sample wall with the spacer, a different type of interaction with molecule of interest 791 may be provided at a different area of sample well 708. A sidewall spacer may have a thickness in the range of 3 nm to 30 nm, or any value or range of values within that range. In some embodiments, a sidewall spacer has a thickness of approximately 10 nm. Examples of suitable materials used to form a sidewall spacer include TiO₂, TiN, TION, TaN, Ta₂O₅, Zr₂O₅, and HfO₂. In some embodiments, the sample well structure has bottom surface 712 proximate to waveguide 716 that lacks spacer material on the side walls. The distance between the bottom surface and sidewall spacer may be in the range of 20 nm to 50 nm, or any value or range of values within that range. In this way, bottom surface 712 of the sample well is closer to waveguide 716, thus improving coupling of excitation energy and reducing the impact of the metal stack on optical loss of excitation energy.

According to some embodiments, an integrated device comprises a sample well. In some embodiments, the integrated device comprises a plurality, or an “array,” of sample wells. For example, FIG. 3 depicts a cross-sectional view of integrated device 800 comprising a plurality of sample wells. As shown, integrated device 800 comprises top cladding 818 between waveguide 816 and metal layer 822, where top cladding 818 separates waveguide 816 and metal layer 822 by a maximum distance h_{c}. Top cladding 818 may have one or more regions that have a dimension less than h_{c} and include one or more sample wells. Such a region may be considered an array of suitable size and shape to include one or more sample wells of the integrated device. Integrated device 800 includes array 820 where top cladding 818 separates waveguide 816 and metal layer 822 by a distance that is less than h_{c}. Array 820 may have an area in a plane perpendicular to the view shown in FIG. 3 of any suitable size and shape to include a desired number of sample wells. In some embodiments, array 820 may have a rectangular shape (e.g., square). Array 820 may have a plurality of sample wells, including sample wells 808₁, 808₂, 808₃, 808₄, 808₅, and 808₆. While FIG. 3 depicts six sample wells, the application is not limited in this respect and any suitable number of sample wells may be formed in an array. An array can have any suitable size or shape. In some embodiments, an array is in a trench region.

Aspects of the techniques described herein involve contacting a sample to a surface of an integrated device. As shown in FIG. 3, integrated device 800 contains a plurality of sample wells in an array 820 that may be formed at depressed surface 810₁ of integrated device 800. Accordingly, in some embodiments, a sample comprising fluid transferred from the fluidic device (e.g., cartridge) is contacted to a depressed surface of integrated device 810₁ in an array of the integrated device. In yet other embodiments, a sample is contacted to a surface of an integrated device that is not in depressed region (e.g., not in a trench region). For example, as depicted in FIG. 3, a sample may be contacted to a surface of integrated device 810₂. It should be appreciated that while FIG. 3 depicts a plurality of sample wells in a depressed region (e.g., a bathtub) of an integrated device, an integrated device may comprise a plurality of sample wells without also comprising a depressed region.

Integrated device 800 may include metal layer 822 over top cladding 818. Metal layer 822 may act as a reflector for emission energy emitted by a sample in a sample well and may improve detection of emission energy by reflecting emission energy towards a sensor of the integrated device. Metal layer 822 may act to reduce the background signal due to photons that do not originate within the sample well. Metal layer 822 may comprise one or more sub-layers. Examples of suitable materials to be used as a metal layer include aluminum, titanium, and titanium nitride. Metal layer 822 may have one or more discontinuities corresponding to the etched portions of top cladding 818 to form sample wells 808₁, 808₂, 808₃, 808₄, 808₅, and 808₆. In some embodiments, a plurality of depressed regions (e.g., trench regions) of the type described herein may be formed in an integrated device, for example, to reduce optical loss due to the interaction of the optical mode traveling down waveguide 816 and metal layer 822. In some embodiments, an integrated device includes a depressed region for a single sample well. The integrated device may have multiple depressed regions in the top cladding where each depressed region corresponds to one sample well.

In certain techniques, it is beneficial for a single sample well to comprise a single molecule of interest (e.g., a single sequencing template). Accordingly, in some embodiments, when loading a sample that comprises, for example, a sequencing template, into sample wells by introducing the sample onto an integrated device comprising an array of sample wells, care should be taken to avoid oversaturating the integrated device with a high concentration of the sequencing template. In such embodiments, it is often advisable to load sample wells using samples having a dilute concentration of sequencing template.

While in some embodiments the fluidic receptacle comprises an integrated device, it should be understood that these embodiments are non-limiting. For example, according to certain embodiments, the fluidic receptacle may be used to transfer fluid to a well plate, or to coat a substrate. In some embodiments, the fluidic receptacle may be permanently connected to a module (e.g., a detection module). In such embodiments, the fluidic device may act as a conduit, controlling fluid flow into the module from a source (e.g., directly from a sequencing module). In some, but not necessarily all embodiments, a detection module may be used to perform measurements of a sample in a fluidic receptacle. The measurements of the sample may be performed, for example, while the fluidic receptacle is aligned with and fluidically connected to the fluidic device.

In some embodiments, the fluidic device is or comprises a cartridge. According to certain embodiments, the cartridge of the fluidic device comprises a channel that is fluidically connected to a fluidic receptacle. In some embodiments, a cartridge includes one or more stored reagents (e.g., of a liquid or lyophilized form suitable for reconstitution to a liquid form). For example, the stored reagents of a cartridge may include reagents suitable for processing a desired sample type (e.g., a buffer, or an imaging solution). In some embodiments, a cartridge is a single-use cartridge (e.g., a disposable cartridge) or a multiple-use cartridge (e.g., a reusable cartridge). In some embodiments, a cartridge is configured to receive a user-supplied sample (e.g., of a protein, or of a nucleic acid). The user-supplied sample may be added to the cartridge before or after the fluidic receptacle is received by the fluidic device, e.g., manually by the user or in an automated process.

In some embodiments, a fluidic device (e.g., a cartridge) comprises a base layer having a surface comprising channels. In some embodiments, at least a portion of at least some of the channels have a substantially triangularly-shaped cross-section having a single vertex at a base of the channel and having two other vertices at the surface of the base layer. In some embodiments, at least a portion of at least some of the channels have a surface layer. The surface layer may comprise an elastomer. The surface layer may be configured to substantially seal off a surface opening of the channel.

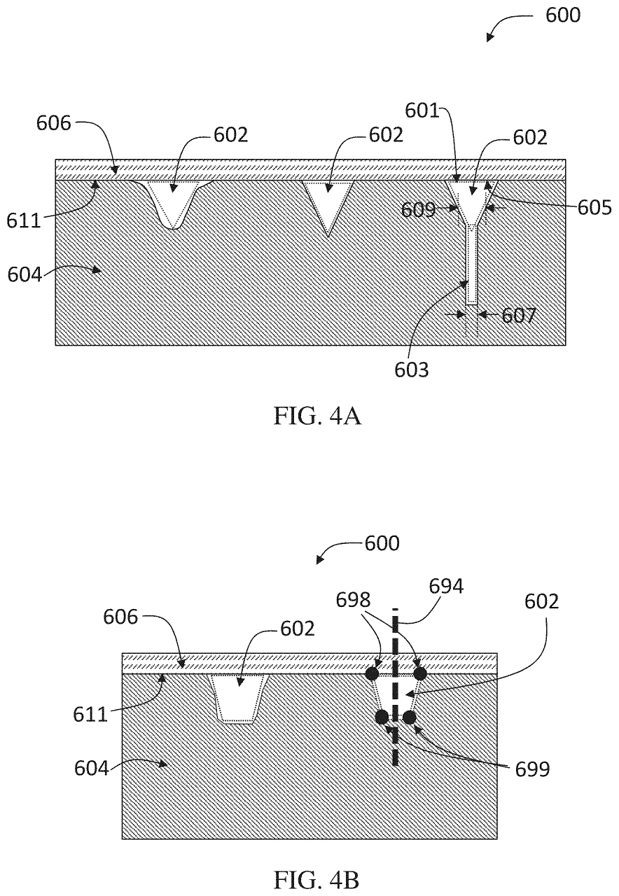

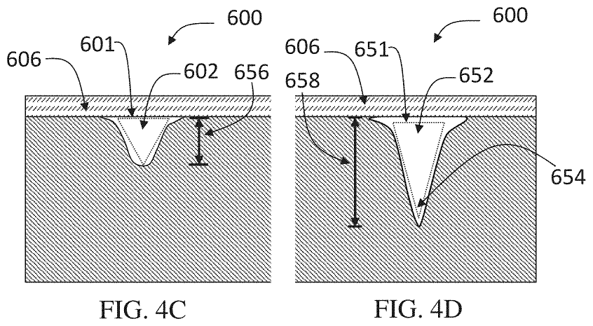

In some embodiments, a fluidic device (e.g., a cartridge) comprises a base layer. In some embodiments, a base layer has a surface comprising one or more channels. For example, FIG. 4A is a schematic diagram of a cross-section view of fluidic device 600 along the width of channels 602, in accordance with some embodiments. The depicted fluidic device 600 includes base layer 604 having surface 611 comprising channels 602. In some embodiments, at least some of the channels are microchannels. For example, in some embodiments, at least some of channels 602 are microchannels. In some embodiments, all of the channels microchannels. For example, referring again to FIG. 4A, in some embodiments, all of channels 602 are microchannels. According to certain embodiments, fluid is transferred from the fluidic device to a fluidic receptacle via a microchannel. For example, referring again to FIGS. 1A-1B, in some embodiments channel 102 is a microchannel.

In some embodiments, a fluidic device (e.g., a cartridge) comprises one or more channels (e.g., microfluidic channels) configured to contain and/or transport a fluid (e.g., a fluid comprising one or more samples, buffers, or imaging solutions) used to load an integrated device. In some embodiments, the fluidic device may also be used for one or more steps of sample preparation. Under such circumstances, the fluidic device may comprise one or more channels configured to contain or transport a fluid (e.g., a fluid comprising one or more reagents). Reagents include buffers, enzymatic reagents, polymer matrices, capture reagents, size-specific selection reagents, sequence-specific selection reagents, and/or purification reagents. In some embodiments, a fluidic device is capable of handling small-volume fluids (e.g., 1-10 microliters, 2-10 microliters, 4-10 microliters, 5-10 microliters, 1-8 microliters, or 1-6 microliters of fluid).

The fluid as described herein may comprise a liquid phase and/or a gas phase. In some embodiments, the fluid comprises a liquid phase. For example, the fluid may comprise an aqueous phase. An aqueous phase may comprise liquid water in an amount of greater than or equal to 50 vol %, greater than or equal to 75 vol %, greater than or equal to 90 vol %, greater than or equal to 95 vol %, greater than or equal to 99 vol %, or 100 vol %). In some embodiments, a liquid phase of a fluid comprises one or more samples, buffers, and/or imaging solutions. According to certain embodiments, a liquid phase of a fluid comprises a solid phase. For example, the fluid may comprise a dispersed solid phase, such as a particulate phase. As a specific example, the liquid phase of the fluid may comprise nanoparticles.

In some embodiments, a fluidic device further comprises a seal plate. In some embodiments, a seal plate comprises a hard plastic, and/or is an injection-molded part. In some embodiments, a seal plate comprises one or more through-holes. In some embodiments, the one or more through-holes have a shape substantially similar to one or more associated channels in the base layer. It should be understood that in this context, the “through-holes” refer to gaps/holes/voids in the seal plate through which one or more mechanical components of, for example, an apparatus, can travel to engage and/or disengage with a surface layer of the fluidic device. For example, a peristaltic pump comprising a roller and a fluidic device (e.g., a cartridge) as described herein may be configured such that the roller travels through at least a portion of the through holes of the seal plate to reach a surface layer of the fluidic device when engaging and/or disengaging with that surface. The through-holes may have any of a variety of shapes and aspect ratios (rectangular, square, circular, oblong, etc.).

In some embodiments, at least some of the one or more through-holes of the seal plate are configured in alignment with one or more associated channels in the base layer. In some embodiments, the fluidic device comprises a surface layer comprising an elastomer disposed between the seal plate and the base layer. In some embodiments, the surface layer is disposed directly between the seal plate in the base layer. In some embodiments, a fluidic device comprises one or more exposed regions of a surface layer disposed between the seal plate and a base layer, wherein each of the one or more exposed regions are defined by an associated through-hole of the seal plate and an aligned channel of the base layer. In some embodiments, one or more exposed portions of the one or more exposed regions of the surface layer can be deformed by a roller to contact one or more associated portions of the walls and/or base of the associated channel of the base layer.

In some embodiments, a fluidic device (e.g., a cartridge) comprises one or more reservoirs configured to receive a fluid and/or contain one or more solutions used in a sample loading process. In some embodiments, at least some channel(s) connect to a reservoir.

The reservoir may be connected to at least some channel(s) at the bottom surface of the channel(s) by intersecting on the perimeter of the reservoir. In some such cases, then, the reservoir and the channels to which it is connected each interface with the surface layer of the fluidic device (e.g., the membrane such as a silicone membrane). However, in some embodiments, the reservoir is connected to at least some channel(s) via a top surface of the reservoir or fluidic device. In some embodiments, the reservoir is empty (e.g., initially empty prior to one or more of the processes herein). For example, the reservoir may initially be empty at the beginning of a loading (or sequencing, analysis or diagnostic) application, but during the application, the sample and/or a reagent (e.g., an imaging solution, a buffer) is added.

According to some embodiments, the reservoir is fluidically connected to an alignment feature (e.g., a first alignment feature) of the fluidic device. In some embodiments, all fluid transported to the fluidic receptacle is transported from a reservoir internal to the fluidic device. However, in some embodiments, at least a portion of the fluid transported to the fluidic receptacle is transported from a reservoir external to the fluidic device (e.g., an external vial, bottle, sample holder, or reservoir of another cartridge). In some embodiments, fluid is transported from the reservoir to the fluidic receptacle via the alignment feature (e.g., the first alignment feature). In some embodiments, the alignment feature (e.g., the first alignment feature) is fluidically connected to both a reservoir internal to the fluidic device and the reservoir external to the fluidic device.

As used herein, the term “channel” will be known to those of ordinary skill in the art and may refer to a structure configured to contain and/or transport a fluid. A channel generally comprises: walls; a base (e.g., a base connected to the walls and/or formed from the walls); and a surface opening that may be open, covered, and/or sealed off at one or more portions of the channel. In some embodiments, a surface portion that is sealed off is completely sealed off. In some embodiments, a surface portion that is sealed off is substantially sealed off. A surface opening may be substantially sealed off if more than 50%, more than 60%, more than 75%, more than 90%, or more than 95% of the surface opening is sealed off. In some embodiments, a surface opening is sealed off by an elastomer.

As used herein, the term “microchannel” refers to a channel that comprises at least one dimension less than or equal to 1000 microns in size. For example, a microchannel may comprise at least one dimension (e.g., a width, a height) less than or equal to 1000 microns (e.g., less than or equal to 100 microns, less than or equal to 10 microns, less than or equal to 5 microns) in size. In some embodiments, a microchannel comprises at least one dimension greater than or equal to 1 micron (e.g., greater than or equal to 2 microns, greater than or equal to 10 microns). Combinations of the above-referenced ranges are also possible (e.g., greater than or equal to 1 micron and less than or equal to 1000 microns, greater than or equal to 10 micron and less than or equal to 100 microns). Other ranges are also possible. In some embodiments, a microchannel has a hydraulic diameter of less than or equal to 1000 microns. As used herein, the term “hydraulic diameter” (DH) will be known to those of ordinary skill in the art and may be determined as: DH=4A/P, wherein A is a cross-sectional area of the flow of fluid through the channel and P is a wetted perimeter of the cross-section (a perimeter of the cross-section of the channel contacted by the fluid).

In some embodiments, at least a portion of at least some channel(s) have a substantially triangularly-shaped cross-section. In some embodiments, at least a portion of at least some channel(s) have a substantially triangularly-shaped cross-section having a single vertex at a base of the channel and having two other vertices at the surface of the base layer. Referring to FIG. 4A, in some embodiments, at least a portion of at least some of channels 602 have a substantially triangularly-shaped cross-section having a single vertex at a base of the channel and having two other vertices at the surface of the base layer.

As used herein, the term “triangular” is used to refer to a shape in which a triangle can be inscribed or circumscribed to approximate or equal the actual shape, and is not constrained purely to a triangle. For example, a triangular cross-section may comprise a non-zero curvature at one or more portions.

A triangular cross-section may comprise a wedge shape. As used herein, the term “wedge shape” will be known by those of ordinary skill in the art and refers to a shape having a thick end and tapering to a thin end. In some embodiments, a wedge shape has an axis of symmetry from the thick end to the thin end. For example, a wedge shape may have a thick end (e.g., surface opening of a channel) and taper to a thin end (e.g., base of a channel), and may have an axis of symmetry from the thick end to the thin end.

Additionally, in some embodiments, substantially triangular cross-sections (i.e., “v-groove(s)”) may have a variety of aspect ratios. As used herein, the term “aspect ratio” for a v-groove refers to a height-to-width ratio. For example, in some embodiments, v-groove(s) has an aspect ratio of less than or equal to 2, less than or equal to 1, or less than or equal to 0.5, and/or greater than or equal to 0.1, greater than or equal to 0.2, or greater than or equal to 0.3. Combinations of the above-referenced ranges are also possible (e.g., between or equal to 0.1 and 2, between or equal to 0.2 and 1). Other ranges are also possible.

In some embodiments, at least a portion of at least some channel(s) have a substantially trapezoidally-shaped cross-section. In some embodiments, at least a portion of at least some channel(s) have a substantially trapezoidally-shaped cross-section having two vertices at a base of the channel and having two other vertices at the surface of the base layer (i.e., the surface opening of a channel). FIG. 4B is similar to FIG. 4A, but presents a cross-sectional diagram of channels 602 having a substantially trapezoidal shape comprising vertices 698 at a surface opening of a channel 602 and vertices 699 at a base of channel 602, according to some embodiments. Generally, in a trapezoidally-shaped cross-section, the two vertices at the surface of the base layer are spaced more widely than the two vertices at the base of the channel, and the two vertices at the base of the channel have an equal depth defining the channel depth. In some embodiments, at least a portion of at least some of channels 602 have a substantially trapezoidally-shaped cross-section.

As used herein, the term “trapezoidal” is used to refer to a shape in which a trapezoid can be inscribed or circumscribed to approximate or equal the actual shape, and is not constrained purely to a trapezoid. For example, a trapezoidal cross-section may comprise a non-zero curvature at one or more portions.

A trapezoid corresponding to a cross-section of a substantially trapezoidally-shaped cross section may be an isosceles trapezoid. In a channel having a substantially isosceles trapezoidally-shaped cross-section, the distance between a first vertex at the base of the channel and a first vertex at the surface of the base layer is equal to the distance between a second vertex at the base of the channel and a second vertex at the surface of the base layer. For example, channels 602 of FIG. 4B have a substantially isosceles trapezoidally-shaped cross-section.

It should, of course, be understood that in addition to the cross-sections already mentioned, the channel may have any of a variety of other suitable cross-section shapes (e.g., semi-circular, rectangular), and that a channel may have different cross-section shapes across different portions of its length.

In some embodiments, the shape of the cross-section of the channel is complementary to that of at least a portion of a roller used to apply pressure the channel (e.g., to drive fluid transport through the channel via, for example, a peristaltic pumping mechanism). For example, in some embodiments, the at least a portion of the cross-section of the roller has a substantially triangular shape (e.g., the roller comprises a wedge portion) and the channel has a substantially triangular shape that is complementary such that the roller can mate with the channel (e.g., via the surface layer such as an elastomeric layer) to establish a well-defined starting position during application of pressure (e.g., for fluid pumping).

In some embodiments, at least a portion of at least some channel(s) have a cross-section comprising a substantially triangular portion and a second portion opening into the substantially triangular portion and extending below the substantially triangular portion relative to the surface of the channel. In some embodiments, the second portion has a diameter (e.g., an average diameter) significantly smaller than an average diameter of the substantially triangular portion. Referring again to FIG. 4A, in some embodiments, at least a portion of at least some of channels 602 have a cross-section comprising substantially triangular portion 601 and second portion 603 opening into substantially triangular portion 601 and extending below substantially triangular portion 601 relative to surface 605 of the channel, wherein second portion 603 has diameter 607 significantly smaller than average diameter 609 of substantially triangular portion 601. In some embodiments a ratio of the diameter of the second portion to the average diameter of the substantially triangular portion is less than or equal to 0.8, less than or equal to 0.6, less than or equal to 0.5, less than or equal to 0.4, less than or equal to 0.3, less than or equal to 0.2, and/or as low as 0.1 or lower. In some such cases, the second portion of a channel having a significantly smaller diameter than that of the average diameter of the substantially triangular portion of the channel can result in the substantially triangular portion being accessible to the roller of the apparatus and deformed portions of the surface layer, but the second portion being inaccessible to the roller and deformed portions of the surface layer. For example, referring again to FIG. 4A, substantially triangular portion 601 of channel 602 is accessible to a roller (not pictured) and deformed portions of surface layer 606, while second portion 603 is inaccessible to the roller and deformed portions of surface layer 606, in accordance with some embodiments. In some such cases, a seal with the surface layer 606 cannot be achieved in portions of the channel 602 having second portion 603, because fluid can still move freely in second portion 603, even when surface layer 606 is deformed by a roller such that it fills substantially triangular portion 601 but not second portion 603. In some embodiments, a portion along a length of a channel has both a substantially triangular portion and a second portion (“deep section”), while a different portion along the length of the channel has only the substantially triangular portion. In some such embodiments, when the apparatus (e.g., roller) engages with the portion having both a substantially triangular portion and a second portion (deep section), pump action is not started, because a seal with the surface layer is not achieved. However, as the apparatus engages along the length direction of the channel, when the apparatus deforms the surface layer at the portion of the channel having only a substantially triangular section, pump action begins because the lack of second portion (deep section) at that portion allows for a seal (and consequently a pressure differential) to be created. Therefore, in some cases, the presence and absence of deep sections along the length of the channels of the fluidic device (e.g., cartridge) can allow for control of which portions of the channel are capable of undergoing pump action upon engagement with the apparatus.

Likewise, in some embodiments, at least a portion of at least some channel(s) have a cross-section comprising a substantially trapezoidal portion and a second portion opening into the substantially trapezoidal portion and extending below the substantially trapezoidal portion relative to the surface of the channel. In some embodiments, the second portion has a diameter (e.g., an average diameter) significantly smaller than an average diameter of the substantially trapezoidal portion. In some embodiments a ratio of the diameter of the second portion to the average diameter of the substantially trapezoidal portion is less than or equal to 0.8, less than or equal to 0.6, less than or equal to 0.5, less than or equal to 0.4, less than or equal to 0.3, less than or equal to 0.2, and/or as low as 0.1 or lower. In some such cases, the second portion of a channel having a significantly smaller diameter than that of the average diameter of the substantially trapezoidal portion of the channel can result in the substantially trapezoidal portion being accessible to the roller of the apparatus and deformed portions of the surface layer, but the second portion being inaccessible to the roller and deformed portions of the surface layer. In some embodiments, a portion along a length of a channel has both a substantially trapezoidal portion and a second portion (“deep section”), while a different portion along the length of the channel has only the substantially trapezoidal portion. In some such embodiments, when the apparatus (e.g., roller) engages with the portion having both a substantially trapezoidal portion and a second portion (deep section), pump action is not started, because a seal with the surface layer is not achieved. However, as the apparatus engages along the length direction of the channel, when the apparatus deforms the surface layer at the portion of the channel having only a substantially trapezoidal section, pump action begins because the lack of second portion (deep section) at that portion allows for a seal (and consequently a pressure differential) to be created. Therefore, in some cases, the presence and absence of deep sections along the length of the channels of the fluidic device (e.g., cartridge) can allow for control of which portions of the channel are capable of undergoing pump action upon engagement with the apparatus.

The inclusion of such “deep sections” as second portions of at least some of the channels of the fluidic device (e.g., cartridge) may contribute to any of a variety of potential benefits. For example, such deep sections (e.g., second portion 103) may, in some cases, contribute to a reduction in pump volume in peristaltic pumping processes. In some such cases, pump volume can be reduced by a factor of two or more for higher volume resolution. In some cases, such deep sections may also provide for a well-defined starting point for the pump volume that is not determined by where the roller lands on the channel. For example, the interface between a portion of a channel having both a substantially triangular portion and a second portion (deep section) and a portion of a channel having only a substantially triangular portion can, in some cases, be used as a well-defined starting point for the pump volume, because only fluid occupying the volume of the latter channel portion can be pumped. In some cases, where the rollers lands on the channel may have some error associated depending on any of a variety of factors, such as cartridge registration. The inclusion of deep sections may, in some cases, reduce or eliminate variations in pump volume associated with such error.

Similar improvements may be realized by using the more general method of including channels with different depths across different portions of their length. A channel may comprise one, two, three, four, five, or more different cross-sections across different portions of the length of the channel. Different cross-sections may have different depths and/or different shapes. Different cross-sections of the channel may have the same depth, even if they have different shapes.

The present disclosure provides, in some instances, channels having a shallow portion along the length of the channel and having a deep portion along the length of the channel. The terms “shallow portion” and “deep portion” are used to conveniently convey the relative dimensional relationships between the shallow portion and deep portion, and are not meant to imply any particular absolute dimensions. In some embodiments, a shallow portion along a length of a channel has a first cross-section with a first depth, and a deep portion along the length of the channel has a second cross-section with a second depth that is greater than the first depth. The first depth (i.e., the depth of the shallow portion) may be, for example, less than or equal to 85%, less than or equal to 70%, less than or equal to 50%, less than or equal to 25%, and/or as low as 15%, as low as 10%, as low as 5%, or less of the second depth (i.e., the depth of the deep portion). For example, FIG. 4C is a schematic diagram of a cross-sectional view of fluidic device 600, that shows first portion 601 of shallow channel portion 602 having first depth 656, and FIG. 4D is a schematic diagram of a cross-sectional view of fluidic device 600 that shows second portion 651 of deep channel portion 652, having second depth 658. First depth 656 is less than or equal to 50% of second depth 658, as shown. It should be understood, of course, that the channel may comprise additional portions across its length, and that these additional portions may comprise cross-sections that are shallower, deeper, or of identical depth to the shallow channel portion and/or the deep channel portion

The portion of the shallow channel may have an average diameter that is substantially similar to the average diameter of the portion of the deep channel. For example, the ratio of the average diameter of the portion of the shallow channel to the average diameter of the portion of the deep channel is greater than or equal to 0.85, greater than or equal to 0.9, greater than or equal to 0.95, greater than or equal to 1, and/or less than or equal to 1.15, less than or equal to 1.1, less than or equal to 1.05, or less, in some embodiments. Combinations of these ranges (e.g., greater than or equal to 0.85 and less than or equal to 1.15) are also possible. For example, in the channel cross-sections of FIGS. 4C-4D, the channel portions have the same average diameter. In some embodiments, the shallow channel and the deep channel have diameters that are not substantially similar, and the disclosure is not so limited.

In some such cases, the deep channel portion can act similarly to a channel comprising a “deep section,” as discussed above. For example, in some embodiments, fluid can still move freely near a bottom portion of the cross-section of the deep portion. In some cases, a seal with the surface layer 606 cannot be achieved in deep channel portion 652, because fluid can still move freely near bottom portion 654 of the deep portion 651, even when surface layer 606 is deformed by a roller. In some such embodiments, when the apparatus (e.g., roller) engages with the deep channel portion, pump action is not started, because a seal with the surface layer is not achieved. However, as the apparatus engages along the length direction of the channel, when the apparatus deforms the surface layer at the shallow channel portion, pump action begins because the reduced depth at that portion allows for a seal (and consequently a pressure differential) to be created. Therefore, in some cases, the relative depth of portions along the length of the channels of the fluidic device (e.g., cartridge) can allow for control of which portions of the length of the channel are capable of undergoing pump action upon engagement with the apparatus.

The inclusion of channels with different depths across different portions of their length may contribute to any of a variety of potential benefits. For example, deep portions along the channel length (e.g., deep channel portion 652) may, in some cases, contribute to a reduction in pump volume in peristaltic pumping processes. In some such cases, pump volume can be reduced by a factor of two or more for higher volume resolution. In some cases, such deep channel portions may also provide for a well-defined starting point for the pump volume that is not determined by where the roller lands on the channel. For example, the deep channel portion and the shallow channel portion, in some cases, may be used as a well-defined starting point for the pump volume, because only fluid occupying the volume of the latter channel portion can be pumped. In some cases, where the rollers lands on the channel may have some error associated depending on any of a variety of factors, such as cartridge registration. The inclusion of channels having different depths across different portions of their length may, in some cases, reduce or eliminate variations in pump volume associated with such error. Channels including “deep sections,” as described above, may be used as a deep channel portion, in some embodiments. An example of channels with different depths, in combination with other inventive aspects, is provided in greater detail below.

The shallow portion along the length of the channel may comprise greater than or equal to 1%, greater than or equal to 5%, greater than or equal to 10%, greater than or equal to 20%, greater than or equal to 30%, greater than or equal to 50%, or more of the total length of the channel. In some embodiments, the shallow portion of the channel comprises less than or equal to 80%, less than or equal to 50%, or less of the length of the channel. Combinations of these ranges are also possible. For example, the shallow portion along the length of the channel may comprise greater than or equal to 1% and less than or equal to 80% of the length of the channel.

Similarly, the deep portion along the length of the channel may comprise greater than or equal to 1%, greater than or equal to 5%, greater than or equal to 10%, greater than or equal to 20%, greater than or equal to 30%, greater than or equal to 50%, or more of the total length of the channel. In some embodiments, the deep portion of the channel comprises less than or equal to 80%, less than or equal to 50%, or less of the length of the channel. Combinations of these ranges are also possible. For example, the deep portion along the length of the channel may comprise greater than or equal to 1% and less than or equal to 80% of the length of the channel. The length of the shallow portion along the length of the channel and the deep portion along the length of the channel may total 100% of the length of the channel. Of course, since the channel may comprise additional portions along its length, the length of the deep portion along the length of the channel and the length of the shallow portion of the channel do not necessarily total 100% of the length of the channel.

As used herein, an average diameter of a substantially triangular portion of a channel may be measured as an average over the z-axis from the vertex of the substantially triangular portion to the surface of the channel. In the context of this application, the average diameter of a non-substantially triangular cross-section of a channel is an average width of the cross-section perpendicular to a z-axis passing perpendicularly through a surface opening of the channel and intersecting a deepest portion of the channel cross-section. For example, the average diameter of channel 602 in FIG. 4B is the average width relative to z-axis 694, which is perpendicular to the surface opening contacting surface layer 606.

In some embodiments, at least some channels (also referred to herein as pumping lanes) (e.g., all channels) each comprises a valve comprising the surface layer comprising an elastomer. In some embodiments, each valve comprises a blockage in an associated channel formed by the geometry of the end of the channel. For example, the geometry of the end of the channel may be a wall spanning from the bottom of the channel to the top surface of the channel, where the channel interfaces with the surface layer. In some such embodiments, a channel remains closed by its associated valve until enough pressure is applied such that the valve opens. In some embodiments, the valve opens by the surface layer ballooning outward. In some embodiments, each valve is effectively actuated by the roller. For example, in some embodiments, pressure exerted on the surface layer by the roller when the roller is relatively close to the valve causes the surface layer to balloon outward (e.g., like a diaphragm) such that a seal between the small blockage and the surface layer is reversibly broken, thereby allowing fluid to pass through the valve. In some cases, the use of such a “passive” valve can contribute to any of a variety of advantages. For example, in some instances, the use of such an integrated valve described herein can ensure that lanes that are not being pumped (e.g., via engagement with the roller of the apparatus) remain closed. In some such cases, only fluid from channels that are engaged by the apparatus (e.g., pump) is driven from the fluidic device (e.g., cartridge), which can allow for a convenient, simple, and inexpensive way to selectively drive fluids from a multi-channel pump with reduced or no contamination.

In some embodiments, channels have some relatively small width and depth, with an aspect ratio of depth/width of generally less than or equal to 1. In some embodiments, channel width is greater than or equal to 1 mm, greater than or equal to 1.2 mm, greater than or equal to 1.5 mm, less than or equal to 2 mm, less than or equal to 1.8 mm, and/or less than or equal to 1.6 mm. Combinations of the above-referenced ranges are also possible (e.g., between or equal to 1 mm and 2 mm). Other ranges are also possible. In some embodiments, channel depth is greater than or equal to 0.6 mm, greater than or equal to 0.75 mm, greater than or equal to 0.9 mm, less than or equal to 1.5 mm, less than or equal to 1.2 mm, and/or less than or equal to 1.0 mm. Combinations of the above-referenced ranges are also possible (e.g., between or equal to 0.6 mm and 1.5 mm). Other ranges are also possible. In some embodiments, channel aspect ratio is less than or equal to 1, less than or equal to 0.8, less than or equal to 0.6, less than or equal to 0.5, greater than or equal to 0.2, and/or greater than or equal to 0.4. Combinations of the above-referenced ranges are also possible (e.g., between or equal to 0.2 and 1). Other ranges are also possible. In some embodiments, given tolerances and capabilities of a molding process, channels on the order of 1.5 mm wide and on the order of 0.75 mm deep may be appropriate. In some embodiments, a channel cross-section has an aspect ratio of ½ with a 90 degree v-groove which provides both ease of roller access into the channel (e.g., for which a shallower v-groove may be better) and higher volume precision (e.g., for which a deeper v-groove may be better at least because the volume becomes less dependent on achieving precise planarity of the surface layer comprising the elastomer). In some embodiments, the channel depth is on the order of the thickness of the surface layer comprising the elastomer, such that the surface layer can temporarily fill in and seal against imperfections in the channel that are likely to be some significant fraction of the channel dimensions.

In some embodiments, at least a portion of at least some channel(s) have a surface layer. In some embodiments, a surface layer comprises an elastomer. Referring again to FIG. 4A, for example, in some embodiments, at least a portion of at least some of channels 602 have surface layer 606, comprising an elastomer, configured to substantially seal off a surface opening of channel 602. In some embodiments, at least a portion of at least some of channels 602: have a substantially triangularly-shaped cross-section having a single vertex at a base of the channel and having two other vertices at the surface of the base layer; and have surface layer 606, comprising an elastomer, configured to substantially seal off a surface opening of channel 602.

In some embodiments, an elastomer comprises silicone. In some embodiments, the elastomer comprises silicone and/or a thermoplastic elastomer, and/or consists essentially of an elastomer.

In some embodiments, a surface layer is configured to substantially seal off a surface opening of a channel. In some embodiments, a surface layer is configured to completely seal off a surface opening of a channel such that fluid (e.g., liquid) cannot leave the channel except via an entrance or exit of the channel. In some embodiments, a surface layer is bound to a portion of a surface of a base layer (e.g., by an adhesive, by heat lamination, or any other suitable binding means). In some embodiments, a surface layer is bound to a portion of a surface of a base layer by an adhesive. In some embodiments, a surface layer is bound to a portion of a surface of a base layer by heat lamination.

As used herein, the term “seal off” refers to contact at or near the edges of an opening such that the opening is sealed.

As used herein, the term “surface opening” refers to the portion of the channel that would open the channel to a surrounding atmosphere if not covered by a surface layer. For example, a microchannel may have a surface opening.

As used herein, a surface layer may be bound to a portion of the surface of the base layer by any suitable binding means. For example, in some embodiments, a surface layer is bound to a portion of the surface of the base layer covalently, ionically, by Van der Waals interactions, by dipole-dipole interactions, by hydrogen bonding, by pi-pi stacking interactions, or by another suitable bonding means.

In some embodiments, a surface layer is held in tension directly in contact with a portion of a surface of a base layer.

As used herein, a surface (e.g., a ceiling) of a channel may correspond to an inner surface of a surface layer.

In some embodiments, at least a portion of the surface layer is flat in the absence of at least one magnitude of applied pressure. In some embodiments, an entirety of the surface layer is flat in the absence of at least one magnitude of applied pressure. For example, in some embodiments, at least a portion (or an entirety) of the surface layer is flat in the absence of engagement by the roller of the apparatus (which can cause deformation of the surface layer via the application of a pressure).

In some embodiments, at least a portion of at least some channel(s) have walls and a base comprising a material (e.g., a substantially rigid material) that is compatible with biological material. In some embodiments, at least a portion of at least some channel(s) have walls and a base comprising a substantially rigid material. For example, referring again to FIG. 4A, in some embodiments, at least a portion of at least some of channels 602 have walls and a base comprising a substantially rigid material. In some embodiments, a base comprises a material that is the same as the material of base layer 604. In some embodiments, a base comprises a material that is different than the material of base layer 604. For example, a base may comprise a material that is different than the material of base layer 604 in instances where the walls and base of the channel are coated with the rigid material. In some embodiments, the substantially rigid material is compatible with biological material. In some embodiments, the base layer is an injection-molded part.

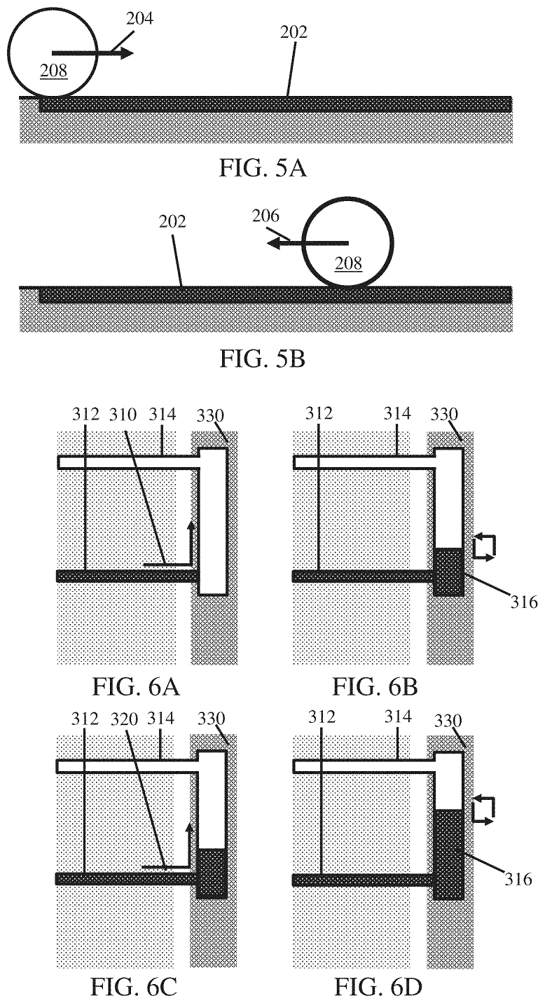

Some aspects are directed toward methods of mixing. Some embodiments comprise translating a roller across at least a portion of a surface of a channel (e.g., a microchannel) in a first direction. Some embodiments further comprise translating the roller across at least a portion of the surface of the channel (e.g., the microchannel) in a second direction, different from the first direction. The second direction may be opposite to the first direction, for example. In some embodiments, the channel (e.g., the microchannel) contains a quantity of fluid. Thus, the translation of the roller may result in a flow of the quantity of the fluid in the channel. In some embodiments, the channel (e.g., the microchannel) is directly connected to an open outlet of the fluidic system. For example, the channel may be connected to an open vent or waste outlet. For example, FIG. 5A presents an exemplary cross-sectional illustration of the translation of roller 208 across a surface of channel 202 in first direction 204, according to some embodiments. Meanwhile, FIG. 5B presents an exemplary cross-sectional illustration of the translation of roller 208 across the surface of channel 202 in second direction 206, which is opposite to direction 204, in some embodiments.

According to some embodiments, pressure is applied during the translation of the roller. Pressure is applied during the translation of the roller in the first direction, according to some embodiments. During the first translation of the roller, pressure causes fluid within the channel to flow in a first direction of fluid flow, according to certain embodiments. In some embodiments, pressure is applied during the translation of the roller in the second direction. During the second translation of the roller, pressure causes fluid within the channel to flow in a second direction of fluid flow, according to certain embodiments. Within the channel, the flow of fluid may be caused by a peristaltic mechanism as the roller is translated. According to some embodiments, pressure is maintained between the translating the roller of the first direction in the translating the roller in the second direction. In some embodiments, for example, the roller maintains contact with the surface between the translating the roller in the first direction and the translating the roller in the second direction. In some embodiments, the applied pressure is constant during the translating the roller in the first direction. In some embodiments, the applied pressure stays within 10%, within 5%, within 2%, within 1%, within 0.5% or closer to an average applied pressure during the translating the roller in the first direction. In some embodiments, the applied pressure is constant during the translating the roller in the second direction. In some embodiments, the applied pressure stays within 10%, within 5%, within 2%, within 1%, within 0.5% or closer to an average applied pressure during the translating the roller in the second direction. In some embodiments, an average applied pressure during the translating the roller in the first direction is within 10%, within 5%, within 2%, within 1%, within 0.5% or closer to an average applied pressure during the translating the roller in the second direction. The average applied pressure during translation of the roller over a period of 10 seconds, for example, may be calculated by integrating the applied pressure over the period of 10 seconds and dividing the result by 10 seconds. By first flowing fluid in a first direction, and second flowing fluid in a second direction within the channel in the manner described, it may be possible to agitate fluid within a reservoir (e.g., an interior of a fluidic receptacle) fluidically connected to the channel, according to certain embodiments. Advantageously, maintaining pressure between translating the roller in the first direction and translating the roller in the second direction may result in the mixing of fluid within the reservoir (e.g., an interior of a fluidic receptacle), without the generation of net fluid flow.

In some embodiments, such methods of mixing provide advantages over certain existing methods of mixing fluids. For example, when such a method is used to mix fluid within a fluidic receptacle, the mixing may occur without net fluid flow from the channel. A lack of net fluid flow may reduce sample loss and provided the fluidic receptacle with a longer duration of exposure to a sample (e.g., a peptide, a nucleic acid). In this way, the method of mixing may advantageously increase the yield of a reaction binding the sample to the fluidic receptacle.

In another aspect, a method comprises transferring a first quantity of fluid into a fluidic receptacle. In some embodiments, the first quantity of fluid is transferred from a fluidic device. In some embodiments, the fluid in the fluidic receptacle is mixed, e.g., for a first time. Next, in some embodiments, a second quantity of fluid is transferred into the fluidic receptacle. In some embodiments, the second quantity of fluid is transferred from the fluidic device. The fluid in the fluidic receptacle may then be mixed again, e.g., for a second time. According to certain embodiments, consecutive steps of transferring a quantity of fluid into the fluidic receptacle, followed by mixing of the fluid in the fluidic receptacle, can advantageously facilitate the interaction of the sample with the fluidic receptacle, resulting in an enhanced binding of the sample of the sample to the fluidic receptacle. This may reduce a prerequisite quantity or concentration required for subsequent analysis (e.g., sequencing), according to certain embodiments.

A step of transferring a quantity of fluid and a step of mixing the fluid in the fluidic receptacle may be performed consecutively. In some embodiments, the step of transferring the first quantity of fluid and the first step of mixing the fluid in the fluidic receptacle are performed consecutively. In some embodiments, the step of transferring the second quantity of fluid and the second step of mixing the fluid in the fluidic receptacle are performed consecutively. The method may further comprise additional steps of: transferring an additional quantity of fluid into the fluidic receptacle; and mixing the fluid in the fluidic receptacle. For example, according to certain embodiments, the method comprises at least N steps of transferring an additional quantity of the fluid, wherein between each consecutive pair of steps of transferring a quantity of fluid, the method further comprises mixing the fluid in the fluidic receptacle for an additional time. In some embodiments, the number of additional transfer steps, N, is greater than or equal to 1, 2, 3, 4, 5, 8, 10, 20, 50, 100, 200, 500, 1000, or more.

In some embodiments, the fluidic receptacle has a volume exceeding a volume of a quantity of fluid transferred according to the method. For example, the volume of the fluidic receptacle may exceed a volume of the first quantity and/or the second quantity of fluid transferred. In some embodiments, the volume of the first quantity of the fluid is within 10%, 5%, 1%, 0.5%, or less of the volume of the second quantity of the fluid. In some embodiments, the first quantity of the fluid and the second quantity of the fluid have the same volume. In some embodiments, additional quantities of the fluid have a volume that is within 10%, 5%, 1%, 0.5%, or less of the volume of the first quantity of the fluid. In some embodiments, additional quantities of the fluid have the same volume as the first quantity of the fluid.

The steps of transferring the fluid may be performed by any appropriate method. For example, according to certain embodiments, the fluid is transferred via peristaltic pumping. The peristaltic pumping may be performed using a fluidic device as described herein, in some instances via interfacing with a roller. The mixing of the fluid may be performed using a method of mixing described herein. For example, after a step of transferring a quantity (e.g. a first quantity, a second quantity) of fluid to the fluidic receptacle, the mixing of the fluid in the fluidic receptacle may be performed comprising translating a roller across the surface of a channel (e.g., a channel fluidically connected to the fluidic receptacle) containing a quantity of a fluid (e.g., a third quantity of a fluid; a fourth quantity of a fluid) in a first direction, followed by translating a roller across the surface of the channel in a second direction. In some embodiments, a volume of the quantity of fluid in the channel across which the roller is translated exceeds a volume of the quantity of fluid added to the fluidic receptacle. In some embodiments, mixing using a quantity of fluid greater than or equal to a quantity of transferred fluid may result in better mixing, with a higher degree of association between a sample and an integrated device. For example, a volume of the third quantity of fluid is greater than or equal to a volume of the first quantity of fluid, in some embodiments. As another example, a volume of the fourth quantity of fluid is greater than or equal to a volume of the second quantity of fluid, in some embodiments.

An exemplary representation of a method of mixing is presented in FIGS. 6A-6D. In FIG. 6A, fluidic receptacle 330 is configured to receive a first quantity of fluid from channel 312, according to certain embodiments. The transfer of the fluid is indicated by arrow 310, according to certain embodiments. FIG. 6B represents a subsequent first mixing step, wherein fluid 316 is mixed within fluidic receptacle. The fluid within the fluidic receptacle may be mixed by any appropriate method. For example, fluid 316 may be mixed by translating a roller across a channel (not shown) fluidically connected to channels 312 and/or to channel 314, according to certain embodiments, as described herein. FIG. 6C represents a subsequent second fluid transfer step, indicated by arrow 320, according to certain embodiments. During the mixing, fluid 316 in the fluidic receptacle may be moved partially into and out of the channel, according to certain embodiments, such that no net flow occurs. FIG. 6D represents a subsequent first mixing step, wherein fluid 316 is mixed within fluidic receptacle.

In some embodiments, the fluid described herein comprises a sample. The sample may comprise a molecule, such as a peptide or a nucleic acid, according to certain embodiments. According to certain embodiments, the sample comprises multiple molecules (e.g., multiple species of peptide, multiple species of nucleic acid). According to certain embodiments, the sample has been prepared. Preparing the sample may comprise one or more steps, such as steps of lysing, enriching, digesting (e.g., to form a digested sample), functionalizing (e.g., to form a functionalized sample), and/or purifying the sample.

In some embodiments, one or more molecules (e.g., peptides, nucleic acids) of the sample are functionalized. Functionalization of the molecules of the sample may allow the molecules to be covalently or non-covalently attached to a surface (e.g., a surface of an integrated device). In some embodiments, functionalizing comprises derivatizing an amino acid side chain of the one or more peptides. In some embodiments, functionalizing comprises terminally functionalizing the one or more peptides (e.g., by one or more of the methods described below). In some embodiments, functionalizing one or more peptides of the digested peptide sample forms an unquenched mixture comprising one or more derivatized peptides, which may be subsequently quenched. In some embodiments, reagents are used to covalently modify a moiety of the one or more molecules (e.g., an amino acid side chain). The reagents may comprise imidazole-1-sulfonyl azide, a copper salt (e.g., copper sulfate), and a buffer having a pH of about 10-11

In some embodiments, the reagents comprise a DBCO-labeled DNA-streptavidin conjugate and a buffer, optionally wherein the DBCO-labeled DNA-streptavidin conjugate is immobilized to a surface of an integrated device. In some embodiments, the functionalized peptide is functionalized with a DBCO-labeled DNA-streptavidin conjugate. In some analytical methods (e.g., single molecule analytical methods), a molecule to be analyzed is immobilized onto surfaces such that the molecule may be monitored without interference from other reaction components in solution. In some embodiments, surface immobilization of the molecule allows the molecule to be confined to a desired region of a surface for real-time monitoring of a reaction involving the molecule. For example, in some embodiments, a molecule may be confined to a well of an integrated device.

Accordingly, in some embodiments, the molecule (e.g., a peptide, a nucleic acid) may be immobilized to a surface by attaching any one of the compounds described herein to a surface of a solid support. The solid support may be part of an article (e.g. a fluidic receptacle and/or an integrated device) coupled to or coupleable to a detection module (e.g., sequencing module) downstream of the fluidic devices for sample preparation described herein.

In some embodiments, an immobilization complex-conjugated molecule (e.g., a peptide, a nucleic acid) is contacted to a surface of a solid support (e.g., a fluidic receptacle and/or an integrated device). In some embodiments, the surface is functionalized with a complementary functional moiety configured for attachment (e.g., covalent or non-covalent attachment) to a functionalized terminal end of a molecule (e.g., a peptide, a nucleic acid). In some embodiments, the solid support (e.g., the fluidic receptacle and/or the integrated device) comprises a plurality of sample wells formed at the surface of the solid support as described herein. In some embodiments, the methods comprise immobilizing a single peptide to a surface of each of a plurality of sample wells. In some embodiments, confining a single molecule per sample well is advantageous for single molecule detection methods, e.g., single molecule sequencing.

As used herein, in some embodiments, a surface refers to a surface of a substrate or solid support (e.g., a fluidic receptacle and/or an integrated device). In some embodiments, a solid support refers to a material, layer, or other structure having a surface, such as a receiving surface, that is capable of supporting a deposited material, such as a functionalized peptide described herein. In some embodiments, a receiving surface of a substrate may optionally have one or more features, including nanoscale or microscale recessed features such as an array of sample wells. In some embodiments, an array is a planar arrangement of elements such as sensors or sample wells. An array may be one or two dimensional. A one dimensional array is an array having one column or row of elements in the first dimension and a plurality of columns or rows in the second dimension. The number of columns or rows in the first and second dimensions may or may not be the same. In some embodiments, the array may include, for example, 10², 10³, 10⁴, 10⁵, 10⁶, or 10⁷ sample wells.

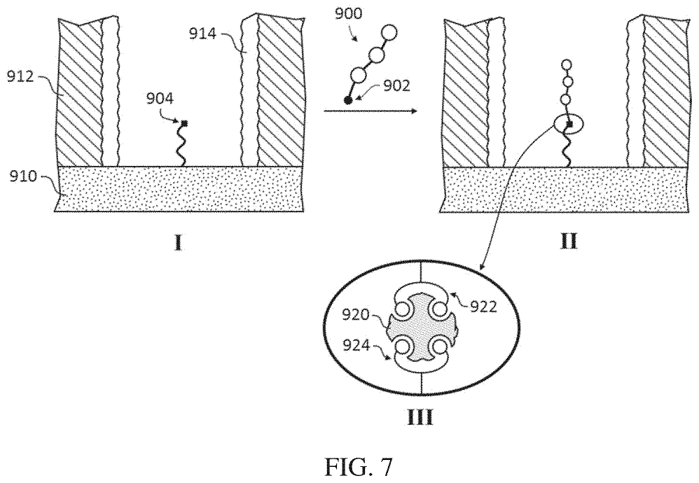

An example scheme of peptide surface immobilization is depicted in FIG. 7. As shown, panels (I)-(II) depict a process of immobilizing peptide 900 that comprises functionalized terminal end 902. In panel (I), an integrated device comprising a sample well is shown. In some embodiments, the sample well is formed by a bottom surface comprising non-metallic layer 910 and side wall surfaces comprising metallic layer 912. In some embodiments, non-metallic layer 910 comprises a transparent layer (e.g., glass, silica). In some embodiments, metallic layer 912 comprises a metal oxide surface (e.g., titanium dioxide). In some embodiments, metallic layer 912 comprises pas sivation coating 914 (e.g., a phosphorus-containing layer, such as an organophosphonate layer). As shown, the bottom surface comprising non-metallic layer 910 comprises complementary functional moiety 904. Methods of selective surface modification and functionalization are described in further detail in U.S. Patent Publication No. 2018-0326412, published Nov. 15, 2018; and U.S. Patent Publication US-2021-0129179-A1, published May 6, 2021, originally filed as U.S. application Ser. No. 17/067,184 on Oct. 9, 2020, the contents of each of which are hereby incorporated by reference.

In some embodiments, peptide 900 comprising functionalized terminal end 902 is contacted with complementary functional moiety 904 of the integrated device to form a covalent or non-covalent linkage group. In some embodiments, functionalized terminal end 902 and complementary functional moiety 904 comprise partner click chemistry handles, e.g., which form a covalent linkage group between peptide 900 and the integrated device. In some embodiments, functionalized terminal end 902 and complementary functional moiety 904 comprise non-covalent binding partners, e.g., which form a non-covalent linkage group between peptide 900 and the solid support. Examples of non-covalent binding partners include complementary oligonucleotide strands (e.g., complementary nucleic acid strands, including DNA, RNA, and variants thereof), protein-protein binding partners (e.g., barnase and barstar), and protein-ligand binding partners (e.g., biotin and streptavidin).

In panel (II), peptide 900 is shown immobilized to the bottom surface through a linkage group formed by contacting functionalized terminal end 902 and complementary functional moiety 904. In this example, peptide 900 is attached through a non-covalent linkage group, which is depicted in the zoomed region of panel (III). As shown, in some embodiments, the non-covalent linkage group comprises avidin protein 920. Avidin proteins are biotin-binding proteins, generally having a biotin binding site at each of four subunits of the avidin protein. Avidin proteins include, for example, avidin, streptavidin, traptavidin, tamavidin, bradavidin, xenavidin, and homologs and variants thereof. In some embodiments, avidin protein 920 is streptavidin. The multivalency of avidin protein 920 can allow for various linkage configurations, as each of the four binding sites are independently capable of binding a biotin molecule (shown as white circles).

As shown in panel (III), in some embodiments, the non-covalent linkage is formed by avidin protein 920 bound to first bis-biotin moiety 922 and second bis-biotin moiety 924. In some embodiments, functionalized terminal end 902 comprises first bis-biotin moiety 922, and complementary functional moiety 904 comprises second bis-biotin moiety 924. In some embodiments, functionalized terminal end 902 comprises avidin protein 920 prior to being contacted with complementary functional moiety 904. In some embodiments, complementary functional moiety 904 comprises avidin protein 920 prior to being contacted with functionalized terminal end 902.

In some embodiments, functionalized terminal end 902 comprises first bis-biotin moiety 922 and a water-soluble moiety, where the water-soluble moiety forms a linkage between first bis-biotin moiety 922 and an amino acid (e.g., a terminal amino acid) of peptide 900. Water-soluble moieties are described in detail elsewhere herein.

In some embodiments, purifying a protein comprises purification via immunoprecipitation. In some embodiments, immunoprecipitation comprises precipitating a target protein out of sample (e.g., a sample before or after functionalization) using an antibody that specifically binds to the target protein.

Some aspects of the present disclosure are directed towards fluidic devices. The fluidic device may be a modular device that can be operably coupled with a system (e.g., a sample preparation module). In some embodiments, a fluidic device is or comprises a cartridge. Fluidic devices (and/or sample preparation modules) may contain mechanical and electronic and/or optical components which can be used to operate a fluidic device component (e.g., cartridges) as described herein. In some embodiments, the fluidic device operates to achieve and maintain specific temperatures on fluidic device regions (e.g., incubation regions). In some embodiments, the fluidic device components operate to apply specific voltages for specific time durations to electrodes of a fluidic device.

In some embodiments, a fluidic device comprises at least one channel. In some embodiments, the fluidic device comprises a microchannel. In some embodiments, at least a portion of some of the channels of the fluidic device (e.g., cartridge) have a surface comprising an elastomer configured to substantially seal off a surface opening of the channel. In some embodiments, the fluidic device components can operate to move liquids to, from, or between reservoirs and/or channels (e.g., an incubation channel) of a fluidic device. In some embodiments, the fluidic device components can operate to move liquids through channel(s) of a fluidic device, e.g., to, from, or between reservoirs and/or other channels (e.g., an incubation channel) of a fluidic device. In some embodiments, the fluidic device components move liquids via a peristaltic pumping mechanism (e.g., apparatus) that is configured to interact with an elastomeric component (e.g., surface layer comprising an elastomer) associated with a channel of a fluidic device (e.g., a cartridge) to pump fluid through the channel.

In some embodiments, the system comprises a sample preparation module, the sample preparation module comprising a peristaltic pump comprising an apparatus comprising a roller and a fluidic device (e.g., a cartridge). In some embodiments, the sample preparation module comprising a peristaltic pump comprising an apparatus comprising a roller and a crank-and-rocker mechanism connected to the roller. In some embodiments, the system comprises a sample preparation module, the sample preparation module comprising a peristaltic pump comprising a fluidic device (e.g., a cartridge) comprising a base layer having a surface comprising channels, wherein at least a portion of at least some of the channels have a substantially triangularly-shaped cross-section having a single vertex at a base of the channel and having two other vertices at the surface of the base layer. The system may comprise a detection module downstream of the sample preparation module. In some embodiments, the sample preparation region comprises more than one fluidic device. In some embodiments, the system comprises a detection module downstream from the sample preparation region of the system.

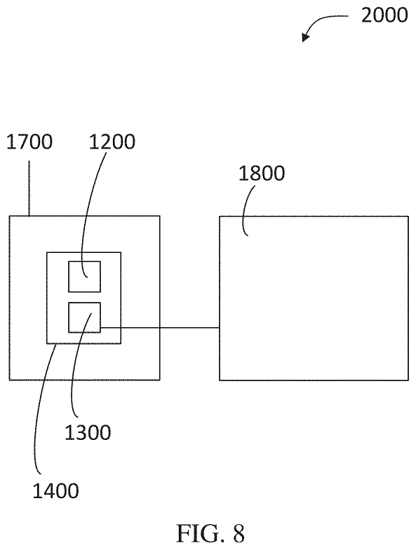

For example, FIG. 8 is a schematic illustration of exemplary system 2000 that incorporates a device (e.g., apparatus, fluidic device, peristaltic pump) described herein, according to some embodiments. Exemplary system 2000 can be used for detecting one or more components of a sample, according to some embodiments. In some embodiments, system 2000 comprises sample preparation module 1700. In some embodiments, system 2000 comprises both sample preparation module 1700 and detection module 1800 downstream of sample preparation module 1700. Exemplary features and associated methods of sample preparation modules and detection modules are described in more detail below. Sample preparation module 1700 and detection module 1800 are configured such that at least a portion of a sample, after being prepared, can be transported (e.g., flowed) from sample preparation module 1700 to detection module 1800 (either directly or indirectly) where the sample is detected (e.g., analyzed, sequenced, identified, etc.), according to some embodiments.

System components can include computer resources, for example, to drive a user interface where sample information can be entered, specific processes can be selected, and run results can be reported. Various aspects and embodiments of fluidic devices and systems are described in detail below.

According to certain embodiments, the steps for preparing the peptide may all be performed manually. However, in some embodiments, one or more steps may be performed automatedly, e.g., using a fluidic device. For example, in some embodiments, a sample preparation device or module is used to prepare a sample for diagnostic purposes. The sample preparation device may comprise one or more cartridges. According to certain embodiments, steps such as digesting (e.g., to form a digested sample), functionalizing (e.g., to form a functionalized sample), and/or purifying the sample may be prepared using separate, fluidically connected fluidic device regions.

In some embodiments, a sample preparation device that is used to prepare a sample for diagnostic purposes is positioned to deliver or transfer to a diagnostic module or diagnostic device a target molecule or a plurality of molecules (e.g., target proteins). For example, in some embodiments, the sample preparation device is configured to deliver or transfer to a fluidic device for loading of a fluidic receptacle, as described herein. In some embodiments, a sample preparation device or module is connected directly to (e.g., physically attached to) or indirectly to a fluidic device. In some embodiments, the transfer of the target molecule is performed using peristaltic pumping (e.g., via a fluidic connection between the sample preparation device and the fluidic device configured to load the fluidic receptacle).

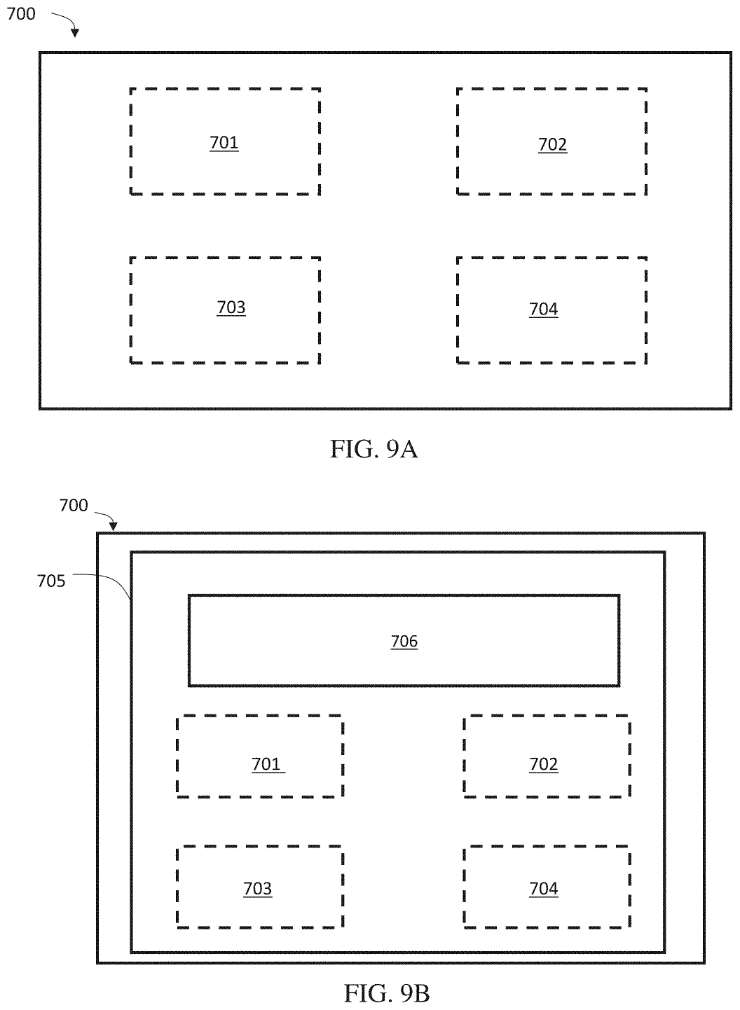

In some embodiments, a system comprises a fluidic device housing that is configured to receive one or more fluidic devices (e.g., configured to receive one cartridge at a time). FIG. 9A shows a schematic diagram of sample preparation device 700, in accordance with some embodiments. A device (e.g., a sample preparation device comprising a cartridge housing) may be configured to receive one or more cartridges (or two or more, or three or more, and so on) either sequentially or simultaneously. Sample preparation device 700, for example, can be configured to receive one or more of lysis cartridge 701, enrichment cartridge 702, fragmentation cartridge 703, and/or functionalization cartridge 704 simultaneously or sequentially.

Samples and reagents may be made to flow (e.g., through channels) in the fluidic devices (e.g., cartridges) described herein via any of a variety of techniques. One such technique is causing flow via peristaltic pumping. In some embodiments, the sample preparation module comprises a pump. In some embodiments, the pump is peristaltic pump. Some such pumps comprise one or more of the inventive components for fluid handling described herein. For example, the pump may comprise an apparatus and/or a fluidic device. In some embodiments, the apparatus of the pump comprises a roller, a crank, and a rocker. In some such embodiments, the crank and the rocker are configured as a crank-and-rocker mechanism that is connected to the roller. The coupling of a crank-and-rocker mechanism with the roller of an apparatus can, in some cases, allow for some of the advantages describe herein to be achieved (e.g., facile disengagement of the apparatus from the fluidic device, well-metered stroke volumes). In some embodiments, the fluidic device of the pump comprises channels (e.g., microfluidic channels). In some embodiments, at least a portion of the channels of the fluidic device have some cross-sectional shapes and/or surface layers that may contribute to any of a number of advantages described herein.

One non-limiting aspect of some fluidic devices (e.g., cartridges) that may, in some cases, provide some benefits is the inclusion of channels having some cross-sectional shapes in the fluidic devices. For example, in some embodiments, the fluidic device comprises v-shaped channels as described herein. One potentially convenient but non-limiting way to form such v-shaped channels is by molding or machining v-shaped grooves into the fluidic device. The recognized advantages of including a v-shaped channel (also referred to herein as a v-groove or a channel having a substantially triangularly-shaped cross-section) in some embodiments in which a roller of the apparatus engages with the fluidic device to cause fluid flow through the channels. For example, in some instances, a v-shaped channel is dimensionally insensitive to the roller. In other words, in some instances, there is no single dimension to which the roller (e.g., a wedge shaped roller) of the apparatus must adhere in order to suitably engage with the v-shaped channel. In contrast, some conventional cross sectional shapes of the channels, such as semi-circular, may require that the roller have some dimension (e.g., radius) in order to suitably engage with the channel (e.g., to create a fluidic seal to cause a pressure differential in a peristaltic pumping process). In some embodiments, the inclusion of channels that are dimensionally insensitive to rollers can result in simpler and less expensive fabrication of hardware components and increased configurability/flexibility.

The sample preparation device may further comprise a pump configured to transport components (e.g., reagents, samples) in the received fluidic devices (e.g., within a channels/reservoirs of a fluidic device or into and/or out of a fluidic device). For example, referring to FIG. 9B, sample preparation device 700 may comprise pump 705 configured to transport components in one or more of lysis cartridge 701, enrichment cartridge 702, fragmentation cartridge 703, and/or functionalization cartridge 704. In some embodiments, a pump comprises an apparatus and a received cartridge, and an interaction between the apparatus of the pump and cartridge causes fluid flow. For example, pump 705 may be a peristaltic pump, and apparatus 706 may operatively couple to a cartridge (e.g., lysis cartridge 701) to cause fluid motion in the cartridge (e.g., when apparatus 706 comprises a roller and lysis cartridge 701 comprises a flexible surface deformable by the roller).

In some aspects, fluidic devices (e.g., cartridges) comprise a surface layer (e.g., a flat surface layer). One exemplary aspect relates to potentially advantageous embodiments involving layering a membrane (also referred to herein as a surface layer) comprising (e.g., consisting essentially of) an elastomer (e.g., silicone) above the v-groove, to produce, in effect, half of a flexible tube. Then, in some embodiments, by deforming the surface layer comprising an elastomer into the channel to form a pinch and by then translating the pinch, negative pressure can be generated on the trailing edge of the pinch which creates suction and positive pressure can be generated on the leading edge of the pinch, pumping fluid in the direction of the leading edge of the pinch. In some embodiments, this pumping by interfacing a fluidic device such as a cartridge (comprising channels having a surface layer) with an apparatus comprising a roller, which apparatus is configured to carry out a motion of the roller that includes engaging the roller with a portion of the surface layer to pinch the portion of the surface layer with the walls and/or base of the associated channel, translating the roller along the walls and/or base of the associated channel in a rolling motion to translate the pinch of the surface layer against the walls and/or base, and/or disengaging the roller with a second portion of the surface layer. In some embodiments, a crank-and-rocker mechanism is incorporated into the apparatus to carry out this motion of the roller.

A conventional peristaltic pump generally involves tubing having been inserted into an apparatus comprising rollers on a rotating carriage, such that the tubing is always engaged with the remainder of the apparatus as the pump functions. By contrast, in some embodiments, channels in fluidic devices (e.g., cartridges) herein are linear or comprise at least one linear portion, such that the roller engages with a horizontal surface. In some embodiments, the roller is connected to a small roller arm that is spring-loaded so that the roller can track the horizontal surface while continuously pinching a portion of the surface layer. Spring loading the apparatus (e.g., a roller arm of the apparatus) can in some cases help regulate the force applied by the apparatus (e.g., roller) to the surface layer and a channel of a fluidic device (e.g., cartridge).

In some embodiments, each rotation of the crank in a crank-and-rocker mechanism connected to the roller provides a discrete pumping volume. In some embodiments, it is straightforward to park the apparatus in a disengaged position, where the roller is disengaged from any fluidic device (e.g., cartridge). In some embodiments, forward and backward pumping motions are fairly symmetrical as provided by apparatuses described herein, such that a similar amount of force (torque) (e.g., within 10%) is required for forward and backward pumping motions.

It may be advantageous to, for a particular size of apparatus, have a relatively high crank radius (e.g., greater than or equal to 2 mm, optionally including associated linkages). Consequently, it may also be advantageous to have a relatively high stroke length (e.g., greater than or equal to 10 mm) to engage with an associated fluidic device (e.g., cartridge). Having relatively high crank radius and stroke length, in some embodiments, ensures no mechanical interference between the apparatus and the fluidic device when moving components of the apparatus relative to the fluidic device.

In some embodiments, having v-shaped grooves advantageously allows for utilization with rollers of a variety of sizes having a wedge-shaped edge. By contrast, for example, having a rectangular channel rather than a v-groove results in the width of the roller associated with the rectangular channel needing to be more controlled and precise in relation to the width of the rectangular channel, and results in the forces being applied to the rectangular channel needing to be more precise. Similarly, the channel(s) having a semicircular cross-section may also require more controlled and precise dimension for the width of the associated roller.

In some embodiments, an apparatus described herein may comprise a multi-axis system (e.g., robot) configured so as to move at least a portion of the apparatus in a plurality of dimensions (e.g., two dimensions, three dimensions). For example, the multi-axis system may be configured so as to move at least a portion of the apparatus to any pumping lane location among associated fluidic device(s). For example, in some embodiments, a carriage herein is functionally connected to a multi-axis system. In some embodiments, a roller is indirectly functionally connected to a multi-axis system. In some embodiments, an apparatus portion, comprising a crank-and-rocker mechanism connected to a roller, is functionally connected to a multi-axis system. In some embodiments, each pumping lane is addressed by location and accessed by an apparatus described herein using a multi-axis system.

In certain embodiments, a sample (e.g., a peptide sample; a nucleic acid sample) is sequenced. For example, a species present in a fluid transferred to a fluidic receptacle may be sequenced. In some embodiments, the sample is first prepared, as described in more detail below. Next, the sample may be transferred to the fluidic receptacle using a fluidic device as described herein. Finally, in some embodiments, the fluidic receptacle (and/or an integrated device of the fluidic receptacle) is introduced to a detection module for sequencing. In some, non-limiting embodiments, the fluidic receptacle is connected to the detection module, such that a prepared sample may be automatedly transported from a sample preparation module to a detection module via a fluidic device as described herein.

Aspects of the instant disclosure involve methods of protein sequencing and identification, methods of protein sequencing and identification, methods of amino acid identification, and compositions, systems, and devices for performing such methods. In some aspects, methods of determining the sequence of a target protein are described. In some embodiments, the target protein is enriched (e.g., enriched using electrophoretic methods, e.g., affinity SCODA) prior to determining the sequence of the target protein. In some aspects, methods of determining the sequences of a plurality of proteins (e.g., at least 2, 3, 4, 5, 10, 15, 20, 30, 50, or more) present in a sample (e.g., a purified sample, a cell lysate, a single-cell, a population of cells, or a tissue) are described. In some embodiments, a sample is prepared as described herein (e.g., digested, lysed, purified, fragmented, and/or enriched for a target protein) prior to determining the sequence of a target protein or a plurality of proteins present in a sample. In some embodiments, a target protein is an enriched target protein (e.g., enriched using electrophoretic methods, e.g., affinity SCODA)

In some embodiments, the instant disclosure provides methods of sequencing and/or identifying an individual protein in a sample comprising a plurality of proteins by identifying one or more types of amino acids of a protein from the mixture. In some embodiments, one or more amino acids (e.g., terminal amino acids) of the protein are labeled (e.g., directly or indirectly, for example using a binding agent) and the relative positions of the labeled amino acids in the protein are determined. In some embodiments, the relative positions of amino acids in a protein are determined using a series of amino acid labeling and cleavage steps. In some embodiments, the relative position of labeled amino acids in a protein can be determined without removing amino acids from the protein but by translocating a labeled protein through a pore (e.g., a protein channel) and detecting a signal (e.g., a Förster resonance energy transfer (FRET) signal) from the labeled amino acid(s) during translocation through the pore in order to determine the relative position of the labeled amino acids in the protein molecule.

In some embodiments, the identity of a terminal amino acid (e.g., an N-terminal or a C-terminal amino acid) is determined prior to the terminal amino acid being removed and the identity of the next amino acid at the terminal end being assessed; this process may be repeated until a plurality of successive amino acids in the protein are assessed. In some embodiments, assessing the identity of an amino acid comprises determining the type of amino acid that is present. In some embodiments, determining the type of amino acid comprises determining the actual amino acid identity (e.g., determining which of the naturally-occurring 20 amino acids an amino acid is, e.g., using a binding agent that is specific for an individual terminal amino acid). However, in some embodiments, assessing the identity of a terminal amino acid type can comprise determining a subset of potential amino acids that can be present at the terminus of the protein. In some embodiments, this can be accomplished by determining that an amino acid is not one or more specific amino acids (i.e., and therefore could be any of the other amino acids). In some embodiments, this can be accomplished by determining which of a specified subset of amino acids (e.g., based on size, charge, hydrophobicity, binding properties) could be at the terminus of the protein (e.g., using a binding agent that binds to a specified subset of two or more terminal amino acids).

In some embodiments, a protein can be digested into a plurality of smaller proteins and sequence information can be obtained from one or more of these smaller proteins (e.g., using a method that involves sequentially assessing a terminal amino acid of a protein and removing that amino acid to expose the next amino acid at the terminus) as described above.

In some embodiments, a protein is sequenced from its amino (N) terminus. In some embodiments, a protein is sequenced from its carboxy (C) terminus. In some embodiments, a first terminus (e.g., N or C terminus) of a protein is immobilized and the other terminus (e.g., the C or N terminus) is sequenced as described herein.

As used herein, sequencing a protein refers to determining sequence information for a protein. In some embodiments, this can involve determining the identity of each sequential amino acid for a portion (or all) of the protein. In some embodiments, this can involve determining the identity of a fragment (e.g., a fragment of a target protein or a fragment of a sample comprising a plurality of proteins). In some embodiments, this can involve assessing the identity of a subset of amino acids within the protein and determining the relative position of one or more amino acid types without determining the identity of each amino acid in the protein). In some embodiments amino acid content information can be obtained from a protein without directly determining the relative position of different types of amino acids in the protein. The amino acid content alone may be used to infer the identity of the protein that is present (e.g., by comparing the amino acid content to a database of protein information and determining which protein(s) have the same amino acid content).

In some embodiments, sequence information for a plurality of protein fragments obtained from a target protein or sample comprising a plurality of proteins (e.g., via enzymatic and/or chemical cleavage) can be analyzed to reconstruct or infer the sequence of the target protein or plurality of proteins present in the sample. Accordingly, in some embodiments, the one or more types of amino acids are identified by detecting luminescence of one or more labeled affinity reagents that selectively bind the one or more types of amino acids. In some embodiments, the one or more types of amino acids are identified by detecting luminescence of a labeled protein.

In some embodiments, the instant disclosure provides compositions, devices, and methods for sequencing a protein by identifying a series of amino acids that are present at a terminus of a protein over time (e.g., by iterative detection and cleavage of amino acids at the terminus). In yet other embodiments, the instant disclosure provides compositions, devices, and methods for sequencing a protein by identifying labeled amino content of the protein and comparing to a reference sequence database.

In some embodiments, the instant disclosure provides compositions, devices, and methods for sequencing a protein by sequencing a plurality of fragments of the protein. In some embodiments, sequencing a protein comprises combining sequence information for a plurality of protein fragments to identify and/or determine a sequence for the protein. In some embodiments, combining sequence information is performed by computer hardware and software. The methods described herein may allow for a set of related proteins, such as an entire proteome of an organism, to be sequenced. In some embodiments, a plurality of single molecule sequencing reactions are performed in parallel (e.g., on a single chip or cartridge) according to aspects of the instant disclosure. For example, in some embodiments, a plurality of single molecule sequencing reactions are each performed in separate sample wells on a single chip or cartridge.

In some embodiments, methods provided herein is used for the sequencing and identification of an individual protein in a sample comprising a plurality of proteins. In some embodiments, the instant disclosure provides methods of uniquely identifying an individual protein in a sample comprising a plurality of proteins. In some embodiments, an individual protein is detected in a mixed sample by determining a partial amino acid sequence of the protein. In some embodiments, the partial amino acid sequence of the protein is within a contiguous stretch of approximately 5-50, 10-50, 25-50, 25-100, or 50-100 amino acids.

Without wishing to be bound by any particular theory, it is expected that most human proteins can be identified using incomplete sequence information with reference to proteomic databases. For example, simple modeling of the human proteome has shown that approximately 98% of proteins can be uniquely identified by detecting just four types of amino acids within a stretch of 6 to 40 amino acids (see, e.g., Swaminathan, et al. PLoS Comput Biol. 2015, 11(2):e1004080; and Yao, et al. Phys. Biol. 2015, 12(5):055003). Therefore, a sample comprising a plurality of proteins can be fragmented (e.g., chemically degraded, enzymatically degraded) into short protein fragments of approximately 6 to 40 amino acids, and sequencing of this protein-based library would reveal the identity and abundance of each of the proteins present in the original sample. Compositions and methods for selective amino acid labeling and identifying proteins by determining partial sequence information are described in in detail in U.S. patent application Ser. No. 15/510,962, filed Sep. 15, 2015, entitled “SINGLE MOLECULE PEPTIDE SEQUENCING,” which is incorporated herein by reference in its entirety.

Sequencing in accordance with the instant disclosure, in some aspects, involves immobilizing a protein (e.g., a target protein) on a surface of a substrate (e.g., of a solid support, for example a chip or cartridge, for example in a sequencing device or module as described herein). In some embodiments, a protein is immobilized on a surface of a sample well (e.g., on a bottom surface of a sample well) on a substrate. In some embodiments, the N-terminal amino acid of the protein is immobilized (e.g., attached to the surface). In some embodiments, the C-terminal amino acid of the protein is immobilized (e.g., attached to the surface). In some embodiments, one or more non-terminal amino acids are immobilized (e.g., attached to the surface). The immobilized amino acid(s) can be attached using any suitable covalent or non-covalent linkage, for example as described in this disclosure. In some embodiments, a plurality of proteins are attached to a plurality of sample wells (e.g., with one protein attached to a surface, for example a bottom surface, of each sample well), for example in an array of sample wells on a substrate.

In some embodiments, the identity of a terminal amino acid (e.g., an N-terminal or a C-terminal amino acid) is determined, then the terminal amino acid is removed, and the identity of the next amino acid at the terminal end is determined. This process may be repeated until a plurality of successive amino acids in the protein are determined. In some embodiments, determining the identity of an amino acid comprises determining the type of amino acid that is present. In some embodiments, determining the type of amino acid comprises determining the actual amino acid identity, for example by determining which of the naturally-occurring 20 amino acids is the terminal amino acid is (e.g., using a binding agent that is specific for an individual terminal amino acid). In some embodiments, the type of amino acid is selected from alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and valine. In some embodiments, determining the identity of a terminal amino acid type can comprise determining a subset of potential amino acids that can be present at the terminus of the protein. In some embodiments, this can be accomplished by determining that an amino acid is not one or more specific amino acids (and therefore could be any of the other amino acids). In some embodiments, this can be accomplished by determining which of a specified subset of amino acids (e.g., based on size, charge, hydrophobicity, post-translational modification, binding properties) could be at the terminus of the protein (e.g., using a binding agent that binds to a specified subset of two or more terminal amino acids).

In some embodiments, assessing the identity of a terminal amino acid type comprises determining that an amino acid comprises a post-translational modification. Non-limiting examples of post-translational modifications include acetylation, ADP-ribosylation, caspase cleavage, citrullination, formylation, N-linked glycosylation, O-linked glycosylation, hydroxylation, methylation, myristoylation, neddylation, nitration, oxidation, palmitoylation, phosphorylation, prenylation, S-nitrosylation, sulfation, sumoylation, and ubiquitination.

In some embodiments, a protein or protein can be digested into a plurality of smaller proteins and sequence information can be obtained from one or more of these smaller proteins (e.g., using a method that involves sequentially assessing a terminal amino acid of a protein and removing that amino acid to expose the next amino acid at the terminus).

In some embodiments, sequencing of a protein molecule comprises identifying at least two (e.g., at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, at least 100, or more) amino acids in the protein molecule. In some embodiments, the at least two amino acids are contiguous amino acids. In some embodiments, the at least two amino acids are non-contiguous amino acids.

In some embodiments, sequencing of a protein molecule comprises identification of less than 100% (e.g., less than 99%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 1% or less) of all amino acids in the protein molecule. For example, in some embodiments, sequencing of a protein molecule comprises identification of less than 100% of one type of amino acid in the protein molecule (e.g., identification of a portion of all amino acids of one type in the protein molecule). In some embodiments, sequencing of a protein molecule comprises identification of less than 100% of each type of amino acid in the protein molecule.

In some embodiments, sequencing of a protein molecule comprises identification of at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100 or more types of amino acids in the protein.

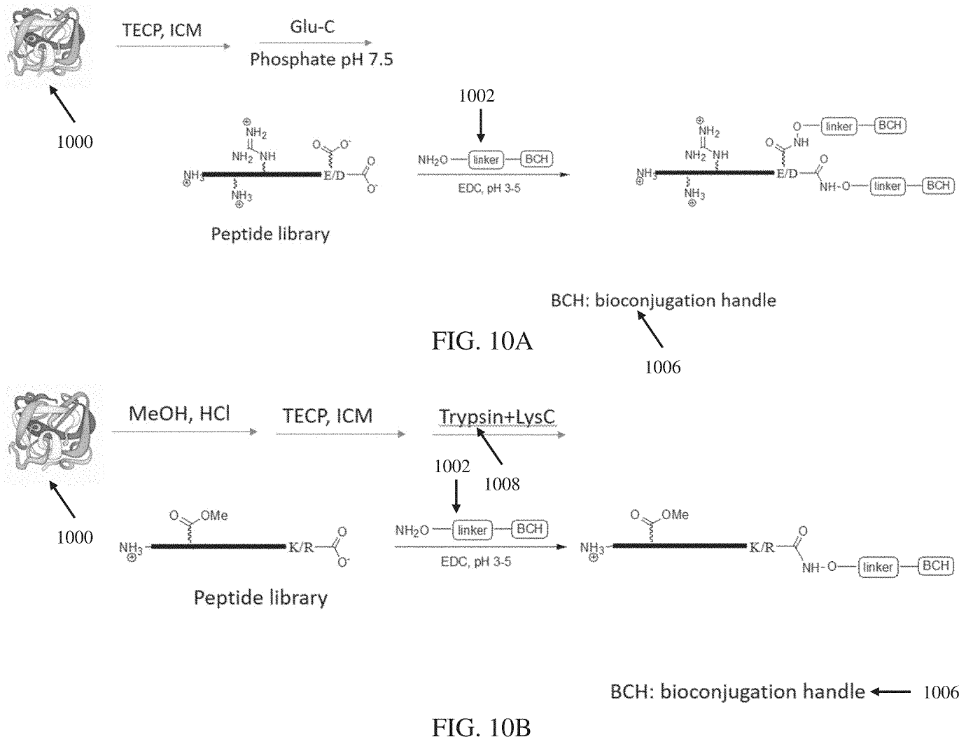

A non-limiting example of protein sequencing by iterative terminal amino acid detection and cleavage is depicted in FIG. 10A. In some embodiments, protein sequencing comprises providing a protein 1000 that is immobilized to a surface 1004 of a solid support (e.g., attached to a bottom or sidewall surface of a sample well) through linkage group 1002. In some embodiments, linkage group 1002 is formed by a covalent or non-covalent linkage between a functionalized terminal end of protein 1000 and a complementary functional moiety of surface 1004. For example, in some embodiments, linkage group 1002 is formed by a non-covalent linkage between a biotin moiety of protein 1000 (e.g., functionalized in accordance with the disclosure) and an avidin protein of surface 1004. In some embodiments, linkage group 1002 comprises a nucleic acid.

In some embodiments, protein 1000 is immobilized to surface 1004 through a functionalization moiety at one terminal end such that the other terminal end is free for detecting and cleaving of a terminal amino acid in a sequencing reaction. Accordingly, in some embodiments, the reagents used in some protein sequencing reactions preferentially interact with terminal amino acids at the non-immobilized (e.g., free) terminus of protein 1000. In this way, protein 1000 remains immobilized over repeated cycles of detecting and cleaving. To this end, in some embodiments, linker 1002 is designed according to a desired set of conditions used for detecting and cleaving, e.g., to limit detachment of protein 1000 from surface 1004. Suitable linker compositions and techniques for functionalizing proteins (e.g., which may be used for immobilizing a protein to a surface) are described in detail elsewhere herein.

In some embodiments, as shown in FIG. 10A, protein sequencing can proceed by (1) contacting protein 1000 with one or more amino acid recognition molecules that associate with one or more types of terminal amino acids. As shown, in some embodiments, labeled amino acid recognition molecule 1006 interacts with protein 1000 by associating with the terminal amino acid.

In some embodiments, the method further comprises identifying the amino acid (terminal amino acid) of protein 1000 by detecting labeled amino acid recognition molecule 1006. In some embodiments, detecting comprises detecting a luminescence from labeled amino acid recognition molecule 1006. In some embodiments, the luminescence is uniquely associated with labeled amino acid recognition molecule 1006, and the luminescence is thereby associated with the type of amino acid to which labeled amino acid recognition molecule 1006 selectively binds. As such, in some embodiments, the type of amino acid is identified by determining one or more luminescence properties of labeled amino acid recognition molecule 1006.

In some embodiments, protein sequencing proceeds by (2) removing the terminal amino acid by contacting protein 1000 with exopeptidase 1008 that binds and cleaves the terminal amino acid of protein 1000. Upon removal of the terminal amino acid by exopeptidase 1008, protein sequencing proceeds by (3) subjecting protein 1000 (having n-1 amino acids) to additional cycles of terminal amino acid recognition and cleavage. In some embodiments, steps (1) through (3) occur in the same reaction mixture, e.g., as in a dynamic peptide sequencing reaction. In some embodiments, steps (1) through (3) are carried out using other methods known in the art, such as peptide sequencing by Edman degradation.

Edman degradation involves repeated cycles of modifying and cleaving the terminal amino acid of a protein, wherein each successively cleaved amino acid is identified to determine an amino acid sequence of the protein. Referring to FIG. 10A, peptide sequencing by conventional Edman degradation can be carried out by (1) contacting protein 1000 with one or more amino acid recognition molecules that selectively bind one or more types of terminal amino acids. In some embodiments, step (1) further comprises removing any of the one or more labeled amino acid recognition molecules that do not selectively bind protein 1000. In some embodiments, step (2) comprises modifying the terminal amino acid (e.g., the free terminal amino acid) of protein 1000 by contacting the terminal amino acid with an isothiocyanate (e.g., PITC) to form an isothiocyanate-modified terminal amino acid. In some embodiments, an isothiocyanate-modified terminal amino acid is more susceptible to removal by a cleaving reagent (e.g., a chemical or enzymatic cleaving reagent) than an unmodified terminal amino acid. In some embodiments, Edman degradation proceeds by (2) removing the terminal amino acid by contacting protein 1000 with an exopeptidase 1008 that specifically binds and cleaves the isothiocyanate-modified terminal amino acid. In some embodiments, exopeptidase 1008 comprises a modified cysteine protease. In some embodiments, exopeptidase 1008 comprises a modified cysteine protease, such as a cysteine protease from Trypanosoma cruzi (see, e.g., Borgo, et al. (2015) Protein Science 24:571-579). In yet other embodiments, step (2) comprises removing the terminal amino acid by subjecting protein 1000 to chemical (e.g., acidic, basic) conditions sufficient to cleave the isothiocyanate-modified terminal amino acid. In some embodiments, Edman degradation proceeds by (3) washing protein 1000 following terminal amino acid cleavage. In some embodiments, washing comprises removing exopeptidase 1008. In some embodiments, washing comprises restoring protein 1000 to neutral pH conditions (e.g., following chemical cleavage by acidic or basic conditions). In some embodiments, sequencing by Edman degradation comprises repeating steps (1) through (3) for a plurality of cycles.

In some embodiments, peptide sequencing can be carried out in a dynamic peptide sequencing reaction. In some embodiments, referring again to FIG. 10A, the reagents required to perform step (1) and step (2) are combined within a single reaction mixture. For example, in some embodiments, steps (1) and (2) can occur without exchanging one reaction mixture for another and without a washing step as in conventional Edman degradation. Thus, in these embodiments, a single reaction mixture comprises labeled amino acid recognition molecule 1006 and exopeptidase 1008. In some embodiments, exopeptidase 1008 is present in the mixture at a concentration that is less than that of labeled amino acid recognition molecule 1006. In some embodiments, exopeptidase 1008 binds protein 1000 with a binding affinity that is less than that of labeled amino acid recognition molecule 1006.

In some embodiments, dynamic protein sequencing is carried out in real-time by evaluating binding interactions of terminal amino acids with labeled amino acid recognition molecules and a cleaving reagent (e.g., an exopeptidase). FIG. 10B shows an example of a method of sequencing in which discrete binding events give rise to signal pulses of a signal output. The inset panel (left) of FIG. 10B illustrates a general scheme of real-time sequencing by this approach. As shown, a labeled amino acid recognition molecule associates with (e.g., binds to) and dissociates from a terminal amino acid (shown here as phenylalanine), which gives rise to a series of pulses in signal output which may be used to identify the terminal amino acid. In some embodiments, the series of pulses provide a pulsing pattern (e.g., a characteristic pattern) which may be diagnostic of the identity of the corresponding terminal amino acid.

As further shown in the inset panel (left) of FIG. 10B, in some embodiments, a sequencing reaction mixture further comprises an exopeptidase. In some embodiments, the exopeptidase is present in the mixture at a concentration that is less than that of the labeled amino acid recognition molecule. In some embodiments, the exopeptidase displays broad specificity such that it cleaves most or all types of terminal amino acids. Accordingly, a dynamic sequencing approach can involve monitoring recognition molecule binding at a terminus of a protein over the course of a degradation reaction catalyzed by exopeptidase cleavage activity.

FIG. 10B further shows the progress of signal output intensity over time (right panels). In some embodiments, terminal amino acid cleavage by exopeptidase(s) occurs with lower frequency than the binding pulses of a labeled amino acid recognition molecule. In this way, amino acids of a protein may be counted and/or identified in a real-time sequencing process. In some embodiments, one type of amino acid recognition molecule can associate with more than one type of amino acid, where different characteristic patterns correspond to the association of one type of labeled amino acid recognition molecule with different types of terminal amino acids. For example, in some embodiments, different characteristic patterns (as illustrated by each of phenylalanine (F, Phe), tryptophan (W, Trp), and tyrosine (Y, Tyr)) correspond to the association of one type of labeled amino acid recognition molecule (e.g., ClpS protein) with different types of terminal amino acids over the course of degradation. In some embodiments, a plurality of labeled amino acid recognition molecules are used, each capable of associating with different subsets of amino acids.

In some embodiments, dynamic peptide sequencing is performed by observing different association events, e.g., association events between an amino acid recognition molecule and an amino acid at a terminal end of a peptide, wherein each association event produces a change in magnitude of a signal, e.g., a luminescence signal, that persists for a duration of time. In some embodiments, observing different association events, e.g., association events between an amino acid recognition molecule and an amino acid at a terminal end of a peptide, can be performed during a peptide degradation process. In some embodiments, a transition from one characteristic signal pattern to another is indicative of amino acid cleavage (e.g., amino acid cleavage resulting from peptide degradation). In some embodiments, amino acid cleavage refers to the removal of at least one amino acid from a terminus of a protein (e.g., the removal of at least one terminal amino acid from the protein). In some embodiments, amino acid cleavage is determined by inference based on a time duration between characteristic signal patterns. In some embodiments, amino acid cleavage is determined by detecting a change in signal produced by association of a labeled cleaving reagent with an amino acid at the terminus of the protein. As amino acids are sequentially cleaved from the terminus of the protein during degradation, a series of changes in magnitude, or a series of signal pulses, is detected.

In some embodiments, signal pulse information are used to identify an amino acid based on a characteristic pattern in a series of signal pulses. In some embodiments, a characteristic pattern comprises a plurality of signal pulses, each signal pulse comprising a pulse duration. In some embodiments, the plurality of signal pulses is characterized by a summary statistic (e.g., mean, median, time decay constant) of the distribution of pulse durations in a characteristic pattern. In some embodiments, the mean pulse duration of a characteristic pattern is between about 1 millisecond and about 10 seconds (e.g., between about 1 ms and about 1 s, between about 1 ms and about 100 ms, between about 1 ms and about 10 ms, between about 10 ms and about 10 s, between about 100 ms and about 10 s, between about 1 s and about 10 s, between about 10 ms and about 100 ms, or between about 100 ms and about 500 ms). In some embodiments, different characteristic patterns corresponding to different types of amino acids in a single protein can be distinguished from one another based on a statistically significant difference in the summary statistic. For example, in some embodiments, one characteristic pattern is distinguishable from another characteristic pattern based on a difference in mean pulse duration of at least 10 milliseconds (e.g., between about 10 ms and about 10 s, between about 10 ms and about 1 s, between about 10 ms and about 100 ms, between about 100 ms and about 10 s, between about 1 s and about 10 s, or between about 100 ms and about 1 s). It should be appreciated that smaller differences in mean pulse duration between different characteristic patterns may require a greater number of pulse durations within each characteristic pattern to distinguish one from another with statistical confidence.

In some embodiments, sequencing of a target peptide comprises identifying at least two (e.g., at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, at least 100, or more) amino acid residues of the target peptide. In some embodiments, the at least two amino acid residues are contiguous amino acid residues. In some embodiments, the at least two amino acid residues are non-contiguous amino acid residues.

In some embodiments, sequencing of a target peptide comprises identification of less than 100% (e.g., less than 99%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 1% or less) of all amino acid residues in the target peptide.

For example, in some embodiments, sequencing of a target peptide comprises identification of less than 100% of one type of amino acid residues in the target peptide. In some embodiments, sequencing of a target peptide comprises identification of less than 100% of each type of amino acid residue in the target peptide.

Some aspects of the instant disclosure further involve sequencing nucleic acids (e.g., deoxyribonucleic acids or ribonucleic acid). In some aspects, compositions, devices, systems, and techniques described herein can be used to identify a series of nucleotides incorporated into a nucleic acid (e.g., by detecting a time-course of incorporation of a series of labeled nucleotides). In some embodiments, compositions, devices, systems, and techniques described herein can be used to identify a series of nucleotides that are incorporated into a template-dependent nucleic acid sequencing reaction product synthesized by a polymerizing enzyme (e.g., RNA polymerase).

Accordingly, also provided herein are methods of determining the sequence of a target nucleic acid. In some embodiments, the target nucleic acid is enriched (e.g., enriched using electrophoretic methods, e.g., affinity SCODA) prior to determining the sequence of the target nucleic acid. In some embodiments, provided herein are methods of determining the sequences of a plurality of target nucleic acids (e.g., at least 2, 3, 4, 5, 10, 15, 20, 30, 50, or more) present in a sample (e.g., a purified sample, a cell lysate, a single-cell, a population of cells, or a tissue). In some embodiments, a sample is prepared as described herein (e.g., lysed, purified, fragmented, and/or enriched for a target nucleic acid) prior to determining the sequence of a target nucleic acid or a plurality of target nucleic acids present in a sample. In some embodiments, a target nucleic acid is an enriched target nucleic acid (e.g., enriched using electrophoretic methods, e.g., affinity SCODA).

In some embodiments, methods of sequencing comprise steps of: (i) exposing a complex in a target volume to one or more labeled nucleotides, the complex comprising a target nucleic acid or a plurality of nucleic acids present in a sample, at least one primer, and a polymerizing enzyme; (ii) directing one or more excitation energies, or a series of pulses of one or more excitation energies, towards a vicinity of the target volume; (iii) detecting a plurality of emitted photons from the one or more labeled nucleotides during sequential incorporation into a nucleic acid comprising one of the at least one primers; and (iv) identifying the sequence of incorporated nucleotides by determining one or more characteristics of the emitted photons.

In another aspect, the instant disclosure provides methods of sequencing target nucleic acids or a plurality of target nucleic acids present in a sample by sequencing a plurality of nucleic acid fragments, wherein the target nucleic acid(s) comprises the fragments. In some embodiments, the method comprises combining a plurality of fragment sequences to provide a sequence or partial sequence for the parent nucleic acid (e.g., parent target nucleic acid). In some embodiments, the step of combining is performed by computer hardware and software. The methods described herein may allow for a set of related nucleic acids (e.g., two or more nucleic acids present in a sample), such as an entire chromosome or genome to be sequenced.

In some embodiments, a primer is a sequencing primer. In some embodiments, a sequencing primer can be annealed to a nucleic acid (e.g., a target nucleic acid) that may or may not be immobilized to a solid support. In some embodiments, a sequencing primer may be immobilized to a solid support and hybridization of the nucleic acid (e.g., the target nucleic acid) further immobilizes the nucleic acid molecule to the solid support (e.g., fluidic receptacle and/or integrated device). In some embodiments, a polymerase (e.g., RNA Polymerase) is immobilized to a solid support (e.g., fluidic receptacle and/or integrated device) and soluble sequencing primer and nucleic acid are contacted to the polymerase. In some embodiments a complex comprising a polymerase, a nucleic acid (e.g., a target nucleic acid) and a primer is formed in solution and the complex is immobilized to the solid support (e.g., via immobilization of the polymerase, primer, and/or target nucleic acid). In some embodiments, none of the components are immobilized to the solid support. For example, in some embodiments, a complex comprising a polymerase, a target nucleic acid, and a sequencing primer is formed in situ and the complex is not immobilized to the solid support.

In some embodiments, sequencing by synthesis methods can include the presence of a population of target nucleic acid molecules (e.g., copies of a target nucleic acid) and/or a step of amplification (e.g., polymerase chain reaction (PCR)) of a target nucleic acid to achieve a population of target nucleic acids. However, in some embodiments, sequencing by synthesis is used to determine the sequence of a single nucleic acid molecule in any one reaction that is being evaluated and nucleic acid amplification may not be required to prepare the target nucleic acid. In some embodiments, a plurality of single molecule sequencing reactions are performed in parallel (e.g., on a single chip or cartridge) according to aspects of the instant disclosure. For example, in some embodiments, a plurality of single molecule sequencing reactions are each performed in separate sample wells (e.g., nanoapertures, reaction chambers) on an integrated device or fluidic receptacle.

In some embodiments, sequencing of a target nucleic acid molecule comprises identifying at least two (e.g., at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, at least 100, or more) nucleotides of the target nucleic acid. In some embodiments, the at least two nucleotides are contiguous nucleotides. In some embodiments, the at least two nucleotides are non-contiguous nucleotides.

In some embodiments, sequencing of a target nucleic acid comprises identification of less than 100% (e.g., less than 99%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 1% or less) of all nucleotides in the target nucleic acid. For example, in some embodiments, sequencing of a target nucleic acid comprises identification of less than 100% of one type of nucleotide in the target nucleic acid. In some embodiments, sequencing of a target nucleic acid comprises identification of less than 100% of each type of nucleotide in the target nucleic acid.

In some embodiments, a system described herein for sample preparation is fluidically connected with an instrument (e.g., a diagnostic instrument) for analyzing at least some of (e.g., all of) the samples prepared by the system. A peptide sample (e.g., a purified peptide sample) may be automatedly transported from the sample preparation module to the diagnostic instrument. In some embodiments, the diagnostic instrument generates an output based on the presence or absence of a band or color based on the underlying sequence of a sample. It should be understood that when components (e.g., modules, devices) are described as being connected (e.g., functionally connected), the connections may be permanently connected, or the connections may be reversibly connected. In some instances, components being described as being connected are decoupleably connected, in that they may be connected (e.g., with a fluidic connection via, for example, a channel, tube, conduit) during a first period of time, but then during a second period of time, they may not be connected (e.g., by decoupling the fluidic connection). In some such embodiments, reversible/decoupleable connections may provide for modular systems in which some components can be replaced or reconfigured, depending on the type of sample preparation/analysis/sequencing/identification being performed. In some embodiments, fluidic connections are connected to components (e.g., modules, devices, fluidic devices and/or fluidic device portions) via a fluidic coupling to a fluidic connector of the component. A few non-limiting examples of a fluidic connector include a protruding tube, an inlet, an outlet, or a nozzle.

In some embodiments, a system herein comprising a sample preparation module further comprises a sequencing module. In some embodiments, a system that comprises a sample preparation module and a sequencing module involves a sequencing chip or cartridge that is embedded into a sample preparation cartridge, such that the two cartridges comprise a single, inseparable consumable. In some embodiments, the sequencing chip or cartridge requires consumable support electronics (e.g., a PCB substrate with wirebonds, electrical contacts). The consumable support electronics may be in direct physical contact with the sequencing chip or cartridge. In some embodiments, the sequencing chip or cartridge requires an interface for a peristaltic pump, temperature control and/or electrophoresis contacts. These interfaces may allow for precise geometric registration for the many electrical contacts and laser alignment. In some embodiments, different sections of a chip or cartridge may comprise different temperatures, physical forces, electrical interfaces of varying voltage and current, vibration, and/or competing alignment requirements. In some embodiments, disparate instrument sub-systems associated with either the sample preparation or sequencing module must be in close proximity in order to share resources. In some embodiments, a system that comprises a sample preparation module and a sequencing module is hands-free (i.e., can be used without the use of hands).

Sequencing of proteins and/or nucleic acids, in accordance with the instant disclosure, in some aspects, may be performed using a system that permits single molecule analysis. The system may include a sequencing module or device and an instrument configured to interface with the sequencing device. As mentioned above, in some embodiments, detection module 1800 comprises such a sequencing module or device. The sequencing module or device may include an array of pixels, where individual pixels include a sample well and at least one photodetector. The sample wells of the sequencing device may be formed on or through a surface of the sequencing device and be configured to receive a sample placed on the surface of the sequencing device. In some embodiments, the sample wells are a component of a fluidic receptacle or of an integrated device that can be inserted into the sequencing device. Collectively, the sample wells may be considered as an array of sample wells. The plurality of sample wells may have a suitable size and shape such that at least a portion of the sample wells receive a single target molecule or sample comprising a plurality of molecules (e.g., a target protein, a target nucleic acid). In some embodiments, the number of molecules within a sample well may be distributed among the sample wells of the sequencing device such that some sample wells contain one molecule (e.g., a target protein, a target nucleic acid) while others contain zero or a plurality of (e.g., two) molecules.

In some embodiments, a sequencing module or device is positioned to receive a target molecule or sample comprising a plurality of molecules (e.g., a target protein, a target nucleic acid) from a sample preparation device. In some embodiments, a sequencing device is connected directly (e.g., physically attached to) or indirectly to a sample preparation device. However, connection between the sample preparation device and the sequencing device or module (or any other type of detection module) is not necessary for all embodiments. In some embodiments, a target molecule (e.g., a target protein, a target nucleic acid) or sample comprising the plurality of molecules is manually transported from the sample preparation device (e.g., sample preparation module) to the sequencing module or device either directly (e.g., without any intervening steps that change the composition of the target molecule or sample) or indirectly (e.g., involving one or more further processing steps that may change the composition of the target molecule or sample). Manual transportation may involve, for example, transport via manual pipetting or suitable manual techniques known in the art.

Excitation light is provided to the sequencing device from one or more light sources external to the sequencing device. Optical components of the sequencing device may receive the excitation light from the light source and direct the light towards the array of sample wells of the sequencing device and illuminate an illumination region within the sample well. In some embodiments, a sample well may have a configuration that allows for the target molecule or sample comprising a plurality of molecules to be retained in proximity to a surface of the sample well, which may ease delivery of excitation light to the sample well and detection of emission light from the target molecule or sample comprising a plurality of molecules. A target molecule or sample comprising a plurality of molecules positioned within the illumination region may emit emission light in response to being illuminated by the excitation light. For example, a protein (or a plurality thereof) or a nucleic acid (or a plurality thereof) may be labeled with a fluorescent marker, which emits light in response to achieving an excited state through the illumination of excitation light. Emission light emitted by a target molecule or sample comprising a plurality of molecules may then be detected by one or more photodetectors within a pixel corresponding to the sample well with the target molecule or sample comprising a plurality of molecules being analyzed. When performed across the array of sample wells, which may range in number between approximately 10,000 pixels to 1,000,000 pixels according to some embodiments, multiple sample wells can be analyzed in parallel.

The sequencing module or device may include an optical system for receiving excitation light and directing the excitation light among the sample well array. The optical system may include one or more grating couplers configured to couple excitation light to the sequencing device and direct the excitation light to other optical components. The optical system may include optical components that direct the excitation light from a grating coupler towards the sample well array. Such optical components may include optical splitters, optical combiners, and waveguides. In some embodiments, one or more optical splitters couples excitation light from a grating coupler and deliver excitation light to at least one of the waveguides. According to some embodiments, the optical splitter has a configuration that allows for delivery of excitation light to be substantially uniform across all the waveguides such that each of the waveguides receives a substantially similar amount of excitation light. Such embodiments may improve performance of the sequencing device by improving the uniformity of excitation light received by sample wells of the sequencing device. Examples of suitable components, e.g., for coupling excitation light to a sample well and/or directing emission light to a photodetector, to include in a sequencing device are described in U.S. Pat. No. 9,885,657, issued Feb. 6, 2018, originally filed as U.S. patent application Ser. No. 14/821,688 on Aug. 7, 2015, titled “INTEGRATED DEVICE FOR PROBING, DETECTING AND ANALYZING MOLECULES,” and U.S. Pat. No. 10,048,208, issued Aug. 14, 2018, issued Aug. 14, 2018, originally filed as U.S. patent application Ser. No. 14/543,865 on Nov. 17, 2014, titled “INTEGRATED DEVICE WITH EXTERNAL LIGHT SOURCE FOR PROBING, DETECTING, AND ANALYZING MOLECULES,” both of which are incorporated herein by reference in their entirety. Examples of suitable grating couplers and waveguides that may be implemented in the sequencing device are described in U.S. Patent Publication No. US-2018-0172906-A1, published Jun. 21, 2018, originally filed as U.S. patent application Ser. No. 15/844,403 on Dec. 15, 2017, titled “OPTICAL COUPLER AND WAVEGUIDE SYSTEM,” which is incorporated herein by reference in its entirety.

Additional photonic structures may be positioned between the sample wells and the photodetectors and configured to reduce or prevent excitation light from reaching the photodetectors, which may otherwise contribute to signal noise in detecting emission light. In some embodiments, metal layers, which may act as a circuitry for the sequencing device, also act as a spatial filter. Examples of suitable photonic structures may include spectral filters, a polarization filters, and spatial filters and are described in U.S. Patent Publication No. US-2019-0025511-A1, published on Jan. 24, 2019, originally filed as U.S. patent application Ser. No. 16/042,968 on Jul. 23, 2018, titled “OPTICAL REJECTION PHOTONIC STRUCTURES,” which is incorporated herein by reference in its entirety.

Components located off of the sequencing module or device may be used to position and align an excitation source to the sequencing device. Such components may include optical components including lenses, mirrors, prisms, windows, apertures, attenuators, and/or optical fibers. Additional mechanical components may be included in the instrument to allow for control of one or more alignment components. Such mechanical components may include actuators, stepper motors, and/or knobs. Examples of suitable excitation sources and alignment mechanisms are described in U.S. Pat. No. 10,246,742, issued Apr. 2, 2019, originally filed as U.S. application Ser. No. 15/161,088 on May 20, 2016, titled “PULSED LASER AND SYSTEM,” which is incorporated herein by reference in its entirety. Another example of a beam-steering module is described in U.S. Pat. No. 10,551,624, issued Feb. 4, 2020, originally filed as U.S. patent application Ser. No. 15/842,720 on Dec. 14, 2017, titled “COMPACT BEAM SHAPING AND STEERING ASSEMBLY,” which is incorporated herein by reference in its entirety. Additional examples of suitable excitation sources are described in U.S. Pat. No. 9,885,657, issued Feb. 6, 2018, originally filed as U.S. patent application Ser. No. 14/821,688 on Aug. 7, 2015, titled “INTEGRATED DEVICE FOR PROBING, DETECTING AND ANALYZING MOLECULES,” which is incorporated herein by reference in its entirety.

The photodetector(s) positioned with individual pixels of the sequencing module or device may be configured and positioned to detect emission light from the pixel's corresponding sample well. Examples of suitable photodetectors are described in U.S. Pat. No. 9,759,658, issued Sep. 12, 2017, originally filed as U.S. patent application No. 14/821,656 on Aug. 7, 2015, titled “INTEGRATED DEVICE FOR TEMPORAL BINNING OF RECEIVED PHOTONS,” which is incorporated herein by reference in its entirety. In some embodiments, a sample well and its respective photodetector(s) may be aligned along a common axis. In this manner, the photodetector(s) may overlap with the sample well within the pixel.

Characteristics of the detected emission light may provide an indication for identifying the marker associated with the emission light. Such characteristics may include any suitable type of characteristic, including an arrival time of photons detected by a photodetector, an amount of photons accumulated over time by a photodetector, and/or a distribution of photons across two or more photodetectors. In some embodiments, a photodetector may have a configuration that allows for the detection of one or more timing characteristics associated with a sample's emission light (e.g., luminescence lifetime). The photodetector may detect a distribution of photon arrival times after a pulse of excitation light propagates through the sequencing device, and the distribution of arrival times may provide an indication of a timing characteristic of the sample's emission light (e.g., a proxy for luminescence lifetime). In some embodiments, the one or more photodetectors provide an indication of the probability of emission light emitted by the marker (e.g., luminescence intensity). Output signals from the one or more photodetectors may then be used to distinguish a marker from among a plurality of markers, where the plurality of markers may be used to identify a sample within the sample. In some embodiments, a sample may be excited by multiple excitation energies, and emission light and/or timing characteristics of the emission light emitted by the sample in response to the multiple excitation energies may distinguish a marker from a plurality of markers.

In operation, parallel analyses of samples within the sample wells are carried out by exciting some or all of the samples within the wells using excitation light and detecting signals from sample emission with the photodetectors. Emission light from a sample may be detected by a corresponding photodetector and converted to at least one electrical signal. The electrical signals may be transmitted along conducting lines in the circuitry of the sequencing device, which may be connected to an instrument interfaced with the sequencing device. The electrical signals may be subsequently processed and/or analyzed. Processing and/or analyzing of electrical signals may occur on a suitable computing device either located on or off the instrument.

The instrument may include a user interface for controlling operation of the instrument and/or the sequencing device. The user interface may be configured to allow a user to input information into the instrument, such as commands and/or settings used to control the functioning of the instrument. In some embodiments, the user interface includes buttons, switches, dials, and/or a microphone for voice commands. The user interface may allow a user to receive feedback on the performance of the instrument and/or sequencing device, such as proper alignment and/or information obtained by readout signals from the photodetectors on the sequencing device. In some embodiments, the user interface provides feedback using a speaker to provide audible feedback. In some embodiments, the user interface includes indicator lights and/or a display screen for providing visual feedback to a user.

In some embodiments, the instrument or device described herein includes a computer interface configured to connect with a computing device. The computer interface may be a USB interface, a FireWire interface, or any other suitable computer interface. A computing device may be any general purpose computer, such as a laptop or desktop computer. In some embodiments, a computing device is a server (e.g., cloud-based server) accessible over a wireless network via a suitable computer interface. The computer interface may facilitate communication of information between the instrument and the computing device. Input information for controlling and/or configuring the instrument may be provided to the computing device and transmitted to the instrument via the computer interface. Output information generated by the instrument may be received by the computing device via the computer interface. Output information may include feedback about performance of the instrument, performance of the sequencing device, and/or data generated from the readout signals of the photodetector.

In some embodiments, the instrument includes a processing device configured to analyze data received from one or more photodetectors of the sequencing device and/or transmit control signals to the excitation source(s). In some embodiments, the processing device comprises a general purpose processor, and/or a specially-adapted processor (e.g., a central processing unit (CPU) such as one or more microprocessor or microcontroller cores, a field-programmable gate array (FPGA), an application-specific integrated circuit (ASIC), a custom integrated circuit, a digital signal processor (DSP), or a combination thereof). In some embodiments, the processing of data from one or more photodetectors is performed by both a processing device of the instrument and an external computing device. In other embodiments, an external computing device is omitted and processing of data from one or more photodetectors is performed solely by a processing device of the sequencing device.

According to some embodiments, the instrument that is configured to analyze target molecules or samples comprising a plurality of molecules based on luminescence emission characteristics detects differences in luminescence lifetimes and/or intensities between different luminescent molecules, and/or differences between lifetimes and/or intensities of the same luminescent molecules in different environments. The inventors have recognized and appreciated that differences in luminescence emission lifetimes can be used to discern between the presence or absence of different luminescent molecules and/or to discern between different environments or conditions to which a luminescent molecule is subjected. In some cases, discerning luminescent molecules based on lifetime (rather than emission wavelength, for example) can simplify aspects of the system. As an example, wavelength-discriminating optics (such as wavelength filters, dedicated detectors for each wavelength, dedicated pulsed optical sources at different wavelengths, and/or diffractive optics) may be reduced in number or eliminated when discerning luminescent molecules based on lifetime. In some cases, a single pulsed optical source operating at a single characteristic wavelength is used to excite different luminescent molecules that emit within a same wavelength region of the optical spectrum but have measurably different lifetimes. An analytic system that uses a single pulsed optical source, rather than multiple sources operating at different wavelengths, to excite and discern different luminescent molecules emitting in a same wavelength region may be less complex to operate and maintain, may be more compact, and may be manufactured at lower cost.

Although analytic systems based on luminescence lifetime analysis may have some benefits, the amount of information obtained by an analytic system and/or detection accuracy may be increased by allowing for additional detection techniques. For example, some embodiments of the systems may additionally be configured to discern one or more properties of a sample based on luminescence wavelength and/or luminescence intensity. In some implementations, luminescence intensity may be used additionally or alternatively to distinguish between different luminescent labels. For example, some luminescent labels may emit at significantly different intensities or have a significant difference in their probabilities of excitation (e.g., at least a difference of about 35%) even though their decay rates may be similar. By referencing binned signals to measured excitation light, it may be possible to distinguish different luminescent labels based on intensity levels.

According to some embodiments, different luminescence lifetimes can be distinguished with a photodetector that is configured to time-bin luminescence emission events following excitation of a luminescent label. The time binning may occur during a single charge-accumulation cycle for the photodetector. A charge-accumulation cycle is an interval between read-out events during which photo-generated carriers are accumulated in bins of the time-binning photodetector. Examples of a time-binning photodetector are described in U.S. Pat. No. 9,759,658, issued Sep. 12, 2017, originally filed as U.S. patent application Ser. No. 14/821,656 on Aug. 7, 2015, titled “INTEGRATED DEVICE FOR TEMPORAL BINNING OF RECEIVED PHOTONS,” which is incorporated herein by reference in its entirety. In some embodiments, a time-binning photodetector generates charge carriers in a photon absorption/carrier generation region and directly transfer charge carriers to a charge carrier storage bin in a charge carrier storage region. In such embodiments, the time-binning photodetector may not include a carrier travel/capture region. Such a time-binning photodetector may be referred to as a “direct binning pixel.” Examples of time-binning photodetectors, including direct binning pixels, are described in U.S. Pat. No. 10,845,308, issued Nov. 24, 2020, originally filed as U.S. patent application Ser. No. 15/852,571 on Dec. 22, 2017, titled “INTEGRATED PHOTODETECTOR WITH DIRECT BINNING PIXEL,” which is incorporated herein by reference in its entirety.

In some embodiments, different numbers of fluorophores of the same type are linked to different components of a target molecule (e.g., a target protein, a target nucleic acid) or a plurality of molecules present in a sample (e.g., a plurality of proteins, a plurality of nucleic acids), so that each individual molecule may be identified based on luminescence intensity. For example, two fluorophores may be linked to a first labeled molecule and four or more fluorophores may be linked to a second labeled molecule. Because of the different numbers of fluorophores, there may be different excitation and fluorophore emission probabilities associated with the different molecule. For example, there may be more emission events for the second labeled molecule during a signal accumulation interval, so that the apparent intensity of the bins is significantly higher than for the first labeled molecule.

The inventors have recognized and appreciated that distinguishing molecules based on fluorophore decay rates and/or fluorophore intensities may facilitate a simplification of the optical excitation and detection systems. For example, optical excitation may be performed with a single-wavelength source (e.g., a source producing one characteristic wavelength rather than multiple sources or a source operating at multiple different characteristic wavelengths). Additionally, wavelength discriminating optics and filters may not be needed in the detection system. Also, a single photodetector may be used for each sample well to detect emission from different fluorophores. The phrase “characteristic wavelength” or “wavelength” is used to refer to a central or predominant wavelength within a limited bandwidth of radiation. For example, a limited bandwidth of radiation may include a central or peak wavelength within a 20 nm bandwidth output by a pulsed optical source. In some cases, “characteristic wavelength” or “wavelength” is used to refer to a peak wavelength within a total bandwidth of radiation output by a source.

In some embodiments, a system comprises a detection module. The detection module (e.g., detection module 1800 in FIG. 8) may be configured to perform any a variety of applications (e.g., bioanalytical applications such as analysis, protein sequencing, peptide sequencing, nucleic acid sequencing, analyte identification, diagnosis). For example, in some embodiments, the detection module comprises an analysis module. The analysis module may be configured to analyze a sample prepared by the sample preparation module. The analysis module may be configured, for example, to determine a concentration of one or more components in a fluid sample. In some embodiments, the detection module comprises a sequencing module. As an example, referring again to FIG. 8, detection module 1800 comprises a sequencing module, according to some embodiments. The sequencing module may be configured to perform sequencing of one or more components of a sample prepared by the sample preparation module. In some embodiments, the identification module is configured to identify peptide molecules (e.g., protein molecules). In some embodiments, the identification module is configured to identify nucleic acid molecules (e.g., protein polynucleotides).

It should be understood that while FIG. 8 depicts shows separate sample preparation module 1700 and detection module 1800 (e.g., analysis module, sequencing module, identification module), the sample preparation module itself (e.g., comprising a peristaltic pump, apparatus, cartridge) may, in some cases, be capable of performing analysis, sequencing, or identification processes. In some embodiments, the sample module is capable of performing a combination of analysis, sequencing, and/or identification processes. For example, in some embodiments, the pump (e.g., pump 1400 that comprises apparatus 1200 and fluidic device 1300) is configured and/or used to deliver some volumes (e.g., relatively small volumes, such as less than or equal to 10 microliters per pump cycle) of sample (e.g., in sequence and/or at some flow rate) directly or indirectly to an integrated detector (e.g., an optical or electrical detector). The integrated detector may be used to make measurements for performing any of a variety of applications (e.g., analysis, sequencing, identification, diagnostics). As such, in some embodiments, a sample (e.g., comprising a peptide, a protein, a nucleic acid, bodily tissue, a bodily secretion) prepared by a system described herein can be sequenced/analyzed using any suitable machine (e.g., a different module, or the same module). In some embodiments, it is advantageous to have a module described herein for sample preparation and a separate machine for detecting (e.g., sequencing) at least some of (e.g., all of) the samples prepared by the system, e.g., so that the machine may be used with minimal downtime (e.g., continuously) for detection (e.g., sequencing) of samples. In some embodiments, a module for sample preparation (e.g., sample preparation module 1700) is fluidically connected with a machine (e.g., a detection module 1800) for detecting (e.g., sequencing) at least some of (e.g., all of) the samples prepared by the system. In some embodiments, a system described herein for sample preparation is fluidically connected with a diagnostic instrument for analyzing at least some of (e.g., all of) the samples prepared by the system. In some embodiments, the diagnostic instrument generates an output based on the presence or absence of a band or color based on the underlying sequence of a sample. It should be understood that when components (e.g., modules, devices) are described as being connected (e.g., functionally connected), the connections may be permanently connected, or the connections may be reversibly connected. In some instances, components being described as being connected are decoupleably connected, in that they may be connected (e.g., with a fluidic connection via, for example, a channel, tube, conduit) during a first period of time, but then during a second period of time, they may not be connected (e.g., by decoupling the fluidic connection). In some such embodiments, reversible/decoupleable connections provide for modular systems in which some components can be replaced or reconfigured, depending on the type of sample preparation/analysis/sequencing/identification being performed.

In another aspect, methods of making a fluidic device (e.g., comprising a cartridge) are provided. In some embodiments, a method of making a fluidic device (e.g. comprising a cartridge) comprises assembling a surface article comprising a surface layer with a base layer to form at least a portion of the fluidic device (e.g. at least a portion of a cartridge). In some embodiments, a method comprises assembling a surface article with a base layer to form a cartridge, wherein assembling comprises, e.g., laser welding, sonic welding, adhering (e.g., using an adhesive), and/or another suitable attachment process for consumables. In certain embodiments, a method comprises aligning the one or more through-holes in the seal plate with corresponding one or more channels in the base layer. In some embodiments, a method comprises over-molding the surface layer comprising the elastomer onto a seal plate comprising one or more through-holes to form the surface article, wherein the surface article further comprises the seal plate.

In some embodiments, the surface layer comprises an elastomer. In some embodiments, the base layer comprises one or more channels. In some embodiments, at least some of the one or more channels have a substantially triangularly-shaped cross-section. Embodiments of methods of making a fluidic device are further described elsewhere herein.

In some embodiments, a method of making a fluidic device (e.g. comprising a cartridge) comprises assembling a surface article comprising a surface layer with a base layer to form at least a portion of the fluidic device. In certain embodiments, the surface layer comprises an elastomer. In certain embodiments, the base layer comprises one or more channels. In certain embodiments, at least some of the one or more channels have a substantially triangularly-shaped cross-section.

In certain embodiments, a method comprises manufacturing one or more mechanical components of a fluidic device (e.g. a cartridge, a discrete fluidic device portion). In some such instances, the manufacturing comprises injection molding (e.g., precision injection molding). In some embodiments, a method comprises injection molding with hard-steel tooling. In certain embodiments, smooth, defect-free surfaces and tight tolerances (e.g., on the order of tens of microns) are attained for one or more mechanical components manufactured by injection molding with hard-steel tooling, which may be advantageous for manufacturing medical device consumables at high throughput.

In certain embodiments, a method comprises manufacturing one or more components of the fluidic device. As one example, in some embodiments, a method comprises manufacturing channels of the fluidic device. As another example, in some embodiments, a method comprises manufacturing reservoirs of the fluidic device. As yet another example, in some embodiments the method comprises manufacturing other regions of the fluidic device. In some embodiments, manufacturing comprises injection molding (e.g., precision injection molding). In some embodiments, method comprises injection molding with hard steel tooling. In certain embodiments, smooth, defect-free surfaces and tight tolerances (e.g., on the order of tens of microns) are attained for one or more mechanical components manufactured by injection molding with hard-steel tooling, which may be advantageous for manufacturing medical device consumables at high throughput.

In some embodiments, a method comprises over-molding a surface layer comprising an elastomer (e.g., silicone, thermoplastic elastomer) onto a seal plate comprising one or more through-holes (e.g., a hard plastic injection-molded part) to form a surface article comprising the surface layer and the seal plate. In some embodiments, a method comprises assembling a surface article with a base layer to form a fluidic device (e.g. comprising a cartridge), wherein assembling comprises, e.g., laser welding, sonic welding, adhering (e.g., using an adhesive), and/or another suitable attachment process for consumables. In certain embodiments, a method comprises aligning the one or more through-holes in the seal plate with corresponding one or more channels in the base layer. For example, the seal plate and the base layer may be aligned such that each through hole in the seal-plate overlaps a corresponding one or more channels in the base layer. In some embodiments, a method comprises over-molding a surface layer (e.g., comprising a relatively soft material) onto a relatively hard material. According to certain embodiments, over-molding comprises molding a first material (e.g., a relatively soft material) against an article (e.g., a first discrete fluidic device portion), such that the first material solidifies on top of the article in a shape determined by the mold. For example, a second discrete fluidic device portion may be over-molded onto a first discrete fluidic device portion. In some embodiments, an over-molded material adheres to the article during its solidification.

In some embodiments, a method comprises die-cutting (e.g., as an alternative to over-molding) a surface layer comprising an elastomer from pre-made sheet stock, which may advantageously offer high precision in durometer and/or thickness. In some embodiments, a method comprises assembling a surface layer comprising an elastomer (e.g., a die-cut elastomeric layer) between a base layer (e.g., comprising and/or consisting essentially of hard plastic) and a seal plate (e.g., comprising and/or consisting essentially of hard plastic) to form a fluidic device (e.g. comprising a cartridge), using, e.g., laser welding, sonic welding, adhering, and/or another suitable attachment process for consumables. In certain embodiments, the base layer comprises one or more channels and the seal plate comprises one or more through-holes. In certain embodiments, a method comprises aligning the one or more through-holes in the seal plate with corresponding one or more channels in the base layer. An exemplary seal-plate comprising through-holes is shown in FIG. 11H (described in greater detail below). As shown, the through-holes shown in FIG. 11H may be aligned with channels 564, shown in FIG. 11G (described in greater detail below) as part of a method of making.

In certain embodiments, the surface layer functions as a peristaltic layer. In certain embodiments, the surface layer functions as a valve diaphragm. In certain embodiments, the surface layer functions as a face-sealing gasket for at least a portion of the fluidic device.

In some embodiments, a method of making a fluidic device (e.g. comprising a cartridge) comprises assembling a surface article comprising a surface layer with a base layer to form the fluidic device. In certain embodiments, the surface layer comprises an elastomer. In certain embodiments, the base layer comprises one or more channels. In certain embodiments, at least some of the one or more channels have a substantially triangularly-shaped cross-section.

In some embodiments, assembling the surface article comprising the surface layer with the base layer to form the fluidic device comprises laser welding, sonic welding, and/or adhering the surface layer to the base layer. For example, in some embodiments, a method comprises adhering the surface layer to the base layer using an adhesive.

In some embodiments, a method comprises die-cutting the surface layer comprising the elastomer from pre-made sheet stock. In some embodiments, the surface article consists essentially of the surface layer. In some embodiments, assembling the surface article comprising the surface layer with the base layer to form the fluidic device (e.g. comprising a cartridge) comprises assembling the surface layer comprising the elastomer between the base layer and a seal plate to form the fluidic device, wherein the seal plate comprises one or more through-holes.

In some embodiments, at least some of the one or more through-holes of a seal plate have a shape substantially similar to the shape of at least some of the one or more channels of the base layer. In some embodiments, a method comprises aligning one or more through-holes in the seal plate with corresponding one or more channels of the base layer. For example, in certain embodiments, aligning one or more through-holes with one or more channels results in one or more exposed regions of the surface layer, corresponding to one or more exposed regions of the surface layer above one or more associated channels in the base layer, such that a roller (e.g., a roller) may deform an exposed portion of an exposed region of the surface layer to contact a portion of the walls and/or base of an associated channel in the base layer.

In some embodiments, a method comprises injection molding one or more mechanical components of a fluidic device. For example, in some embodiments, a method comprises injection molding a cartridge of a fluidic device. In some embodiments, a method comprises injection molding a discrete fluidic device portion (e.g., a discrete fluidic device portion comprising a relatively hard material). According to some embodiments, injection molding one or more mechanical components of the fluidic device comprises injection molding to form the seal plate. In certain embodiments, injection molding one or more mechanical components of the fluidic device (e.g. cartridge) comprises injection molding to form the base layer. Injection molding may comprise, for example, precision injection molding and/or injection molding with hard-steel tooling.

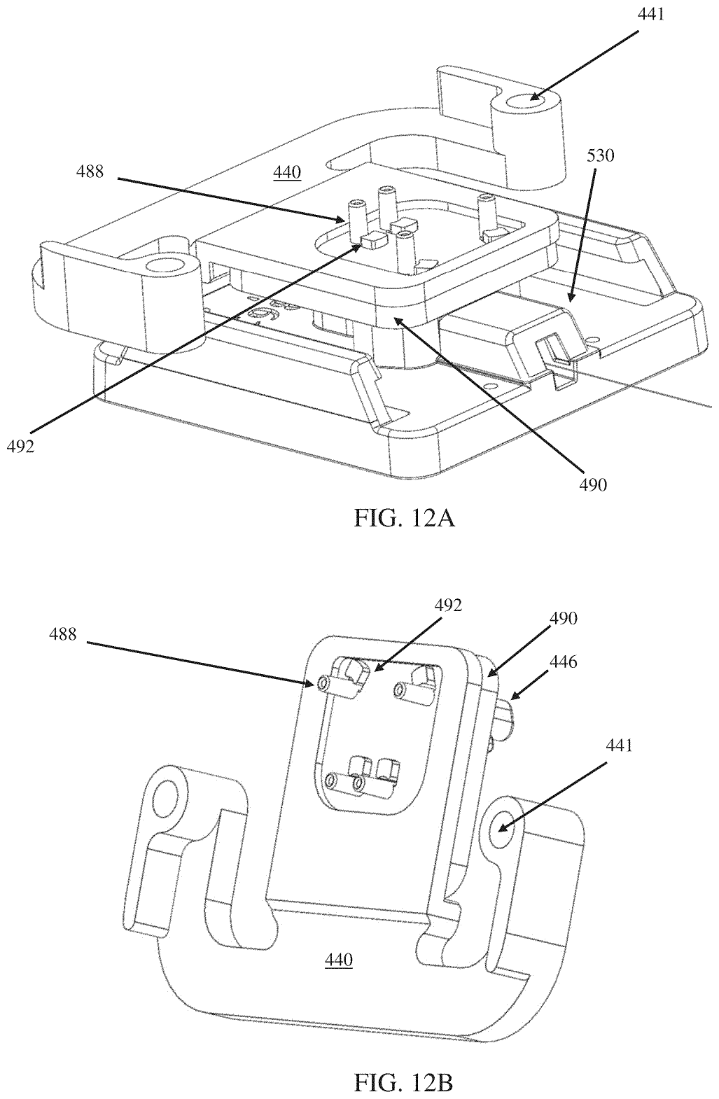

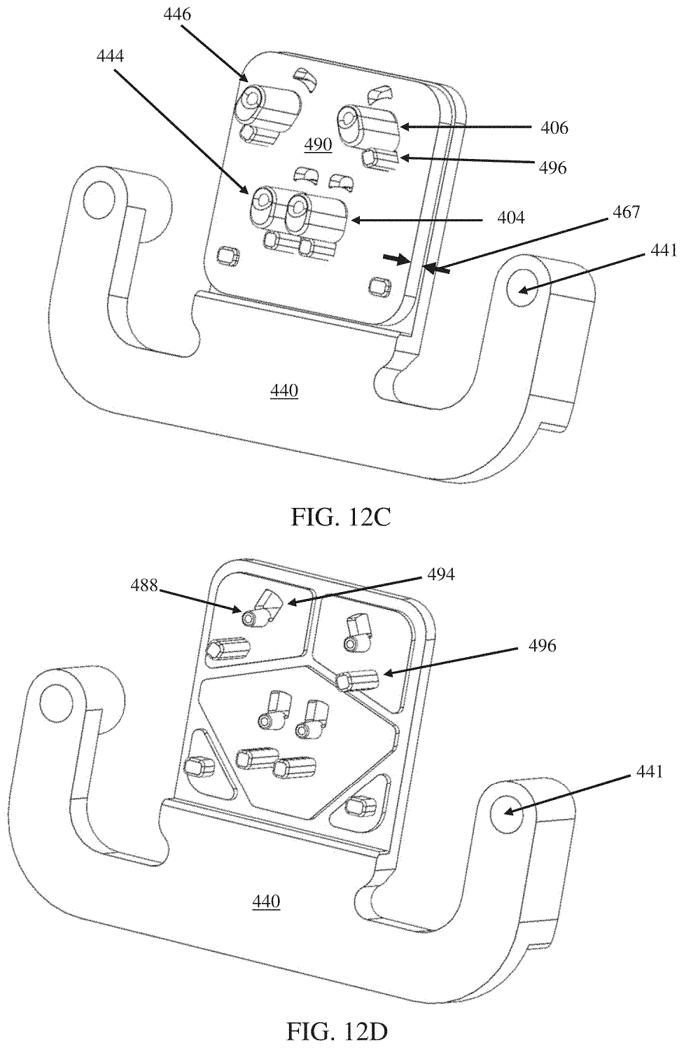

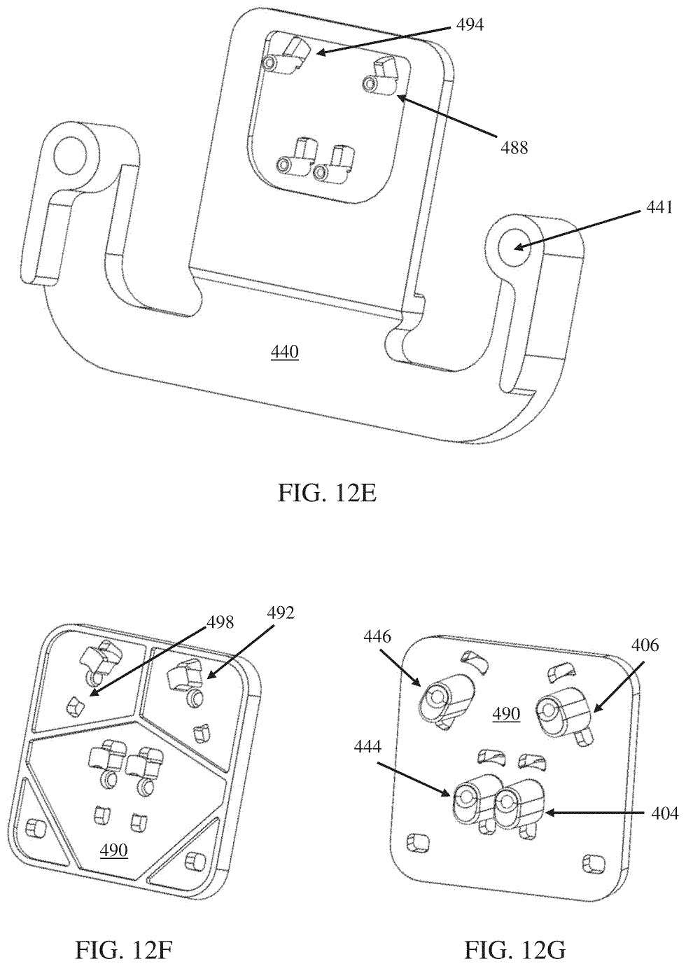

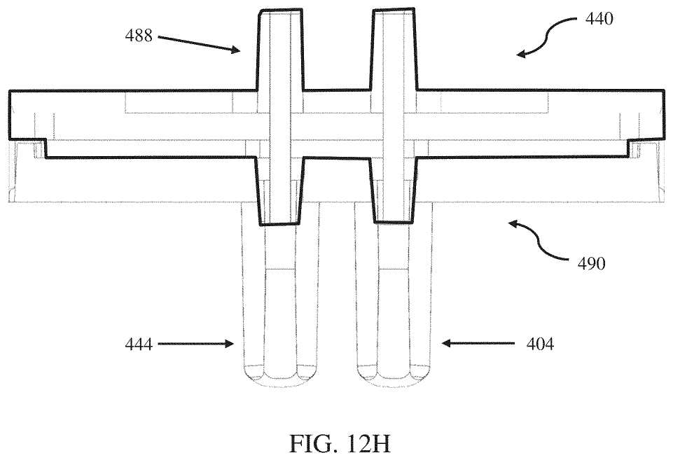

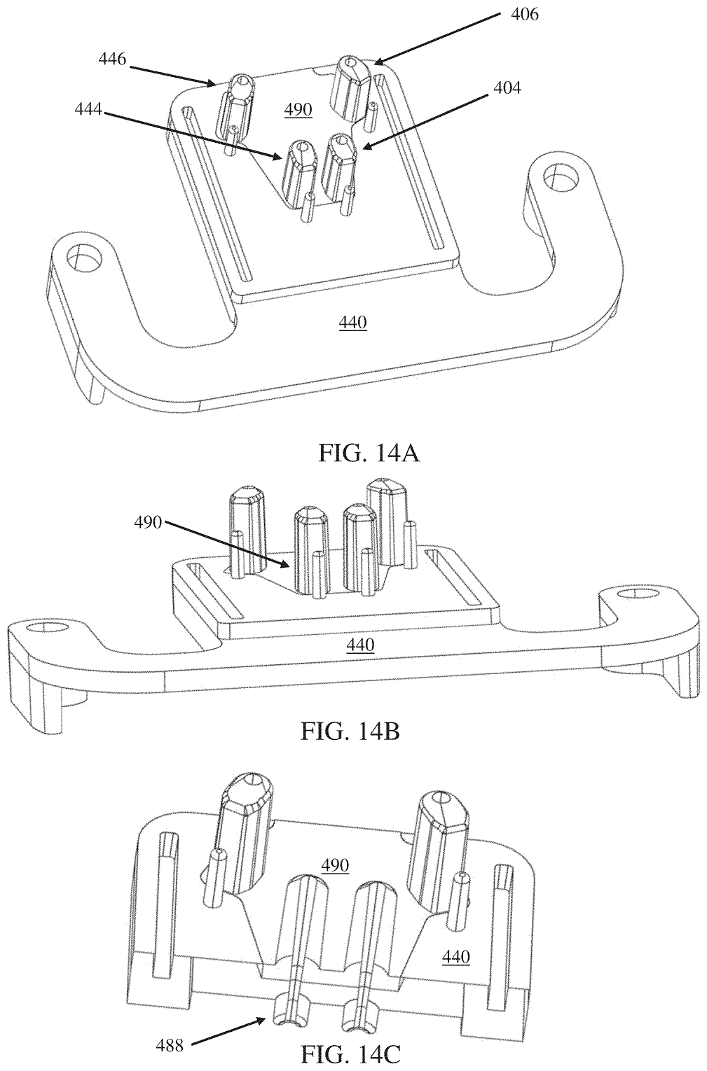

In some cases, a method comprises casting or molding a fluidic device portion into an appropriate geometry. According to certain embodiments, a discrete fluidic device portion (e.g., a first fluidic device portion) is manufactured using methods described above. In some embodiments, a second fluidic device portion may be formed directly on the first fluidic device portion. For example, in some embodiments, the second fluidic device portion is over-molded onto the first fluidic device portion. For an example of an over-molded second fluidic device portion, refer to FIGS. 14A-14C, below. In some embodiments, the second fluidic device portion may be formed separately, and may be mechanically coupled to the first fluidic device portion, e.g., using connection features as described elsewhere herein. For example, in some embodiments the second fluidic device portion may be molded separately, and connected to the first fluidic device portion by aligning soft connectors of the second fluidic device portion with holes of the first fluidic device portion, as described below with reference to FIGS. 12B-12D. An over-molded second discrete fluidic device portion may be advantageous, relative to a separately-formed second discrete fluidic device portion, because it may streamline an assembly process for a fluidic device. Another possible advantage of an over-molded second discrete fluidic device portion is that it may remove certain failure modes that can, in some instances, be associated with a separately-formed second discrete fluidic device portion. For example, over-molded second discrete fluidic device portions may have a reduced risk of leakage. Separately-formed second discrete fluidic device portions may have other advantages, however, and the disclosure is not thus limited.

In some embodiments, a first discrete fluidic device portion and a second discrete fluidic device portion may be assembled into a discrete coupling portion of a fluidic device. The discrete coupling portion may comprise one or more alignment features, e.g., as part of the first fluidic device portion and/or the second fluidic device portion. In some embodiments, an alignment feature of the first fluidic device portion and/or the second fluidic device portion may be fluidically connected to a cartridge of a fluidic device. The cartridge may comprise a surface layer that comprises an elastomer. In some embodiments, the cartridge comprises a base layer that comprises one or more channels. In some embodiments, at least some of the one or more channels of the cartridge have a substantially triangularly-shaped cross-section. Such a cartridge may be made using methods described above, with reference to making complete fluidic devices.

In one aspect, a method, comprises assembling a fluidic system. In some embodiments, a fluidic system is assembled by receiving a fluidic receptacle into a fluidic device. The fluidic receptacle may be received in an alignment with respect to the fluidic device. In some embodiments, a method comprises coupling a first alignment feature of the fluidic device to a third alignment feature, located on the fluidic receptacle. In some embodiments, a method comprises coupling a second alignment feature of the fluidic device to a fourth alignment feature, located on the fluidic receptacle. In some embodiments, the alignment features of the fluidic receptacle are coupled to the alignment features of the fluidic device simultaneously. For example, the alignment features of the fluidic receptacle may be arranged to align with the alignment features of the fluidic device, such that the alignment features can be simultaneously coupled.

FIGS. 11A-11I show various views of a schematic illustration of a fluidic device for transferring fluid to a fluidic receptacle in the form of an integrated device, according to some embodiments. FIG. 11A shows a perspective schematic illustration of fluidic device 501 and fluidic receptacle 530, while FIG. 11B shows an exploded perspective illustration of fluidic device 501 and fluidic receptacle 530, according to some embodiments. FIG. 11C shows, similarly, a perspective schematic illustration of solid substrate 514 of fluidic device 501 comprising recess 516, according to some embodiments. Although the mounting element shown in FIGS. 11A-11I comprises a recess, it should be understood that any type of mounting element may generally be used. As shown in FIG. 11C, mounting element 516 comprises surface 543, which is angled with respect to a lateral dimension of device 501. FIG. 11D shows a side-view schematic illustration of fluidic device 501 and fluidic receptacle 530, according to some embodiments, while FIG. 11E shows a top-view schematic illustration of fluidic device 501 and fluidic receptacle 530, and FIG. 11F shows a bottom-view schematic illustration of fluidic device 501, according to some embodiments. Fluidic device 501, which comprises cartridge 515, comprises solid substrate 514 comprising recess 516 having a surface angled with respect to a lateral dimension of fluidic device 501, in some embodiments. Fluidic device 501, according to some embodiments, is configured to receive a fluidic receptacle 530 into recess 516, and to couple to alignment features 524, 526, 554, and 556, such that fluidic receptacle 530 is fluidically connected to the fluidic device via fluidic connections 512. The mounting element comprising recess 516 further comprises mounting features 539, which are presented as holes configured to receive secondary mounting features 537. In some embodiments, fluidic device 501 comprises reservoirs 532, which may contain samples or reagents, and which are fluidically connected to fluidic receptacle 530 via fluidic connections 512.

According to certain embodiments, a fluidic receptacle may be further coupled to a fluidic device using an optional mechanical coupler. For example, once the fluidic device has received the fluidic receptacle in an alignment, according to certain embodiments, a mechanical coupler may be added to reinforce the coupling between the fluidic receptacle and the fluidic device. For example, in some embodiments, fluidic device 501 further comprises a mechanical coupler 536, which can mechanically support the connection between fluidic receptacle 530 and a discrete fluidic device portion 540, and a handle 538, which may assist with the manual manipulation of discrete fluidic device portion 540. Secondary mounting features 537 are bolts, screws, or other retaining elements configured to pass through holes 541, in some embodiments. Secondary mounting features 537 may at least partially align discrete fluidic device portion 540, relative to fluidic receptacle 530, in some embodiments.

In some embodiments, fluidic device 501 comprises seal plate 560 and elastomer surface 562. FIG. 11G shows a bottom-view schematic illustration of fluidic device 501 with seal plate 560 and elastomer surface 562 stripped away to reveal pumping lanes 564, which are channels, fluidically connected to channels 502, wherein the channels are fluidically connected to the fluidic receptacle through first alignment feature 504, according to some embodiments. FIG. 11H provides a perspective schematic illustration of seal plate 460, according to some embodiments. FIGS. 11I-11J show various perspective schematic illustrations of discrete fluidic device portion 540, according to some embodiments. FIG. 11I shows first alignment feature 504, second alignment feature 506, a fifth alignment feature 544, and a sixth alignment feature 546, according to some embodiments. According to some embodiments, alignment features 504 and 544 are fluidically connected to the channels and alignment features 506 and 546 are fluidically connected to waste outlets of the fluidic device (not shown) via fluidic connections 512, shown in FIG. 11J. FIGS. 11K-11L show various perspective schematic illustrations of fluidic receptacle 530, according to some embodiments. FIG. 11K shows third alignment feature 524, fourth alignment feature 526, sixth alignment feature 554, and eighth alignment feature 556, according to some embodiments. Alignment features 524, 526, 554, and 556 are configured to be coupled to alignment features 504, 506, 544, and 546, respectively, such that fluidic device 501 receives fluidic receptacle 530 in an alignment, in some embodiments. When the alignment features are coupled, according to some embodiments, inlets 510, illustrated in FIG. 11L, and outlets 520, illustrated in FIG. 11L, are connected to the fluidic device via fluidic connections 512 shown in FIG. 11J. FIGS.11M-11N show various perspective schematic illustrations of mechanical coupler 536, according to some embodiments.

An embodiment as described with reference to FIGS. 11A-11N may be used to load an integrated device. According to certain embodiments, a fluid (e.g., a sample prepared in a sample preparation module) is added to at least one of reservoirs 532. The fluid may be added to two of the reservoirs (e.g., one on each side of fluidic device 501). Other reservoirs may be used to hold buffers or other solutions useful for loading the fluidic receptacle. The reservoirs are filled manually, according to certain embodiments, but could also (or instead) be connected to the sample preparation module via a fluidic connection. In some embodiments, the fluidic device may be primed, e.g., by flowing fluid from pumping lanes 564, through channels 502 and fluidic connections 512, and (optionally) into a ‘dummy’ fluidic receptacle. The dummy fluidic receptacle does not comprise an integrated device, according to certain embodiments, because it is only used for priming the fluidic device.

In some embodiments, fluidic receptacle 530 is then coupled to fluidic device 501 as described above. Next, the sample is loaded, via peristaltic pumping of pumping lanes 564 (e.g., via contact from a roller), according to certain embodiments, via alignment features 544 and 504 of fluidic device. Because fluidic receptacle 530 is angled, according to some embodiments, air bubbles present in the fluid may preferentially separate. In some embodiments, fluid is transferred in multiple steps, separated by steps of mixing the fluid in the fluidic receptacle, as described in more detail above. In certain embodiments, once the sample has been loaded to fluidic receptacle 530, excess sample is ejected by transferring an imaging solution to fluidic receptacle 530, forcing the sample fluid through alignment features 506 and 546, where it is directed towards waste outlets of fluidic device 101. The imaging solution is also mixed in fluidic receptacle 530, according to certain embodiments, to remove target molecules that may have bound to a surface of the integrated device non-specifically. Finally, fluidic receptacle 530 may be removed from integrated device 501, e.g., to be transferred to a detection module.

FIGS. 12A-12H show various views of a schematic illustration of first fluidic device portion 440 and second fluidic device portion 490, in accordance with certain embodiments. First fluidic device portion 440 and second fluidic device portion 490 may be configured for transferring fluid to fluidic receptacle 530, which in the embodiment in FIGS. 12A-12H is in the form of an integrated device, according to some embodiments. While fluidic receptacle 530 is identical to the fluidic receptacle of FIGS. 11A-11I, first fluidic device portion 440 and second fluidic device portion 490 differ from fluidic device portion 540 of FIGS. 11A-11I. In this embodiment, the second fluidic device portion comprises a relatively flexible material that helps to seal a fluidic connection between the fluidic device and the fluidic receptacle, as described in greater detail above, while the first fluidic device portion comprises a relatively rigid material, to provide mechanical support.

FIG. 12A presents first fluidic device portion 440 and second fluidic device portion 490, mechanically coupled via connection features 492, in the form of pegs, which are disposed on second fluidic device portion 490 and are mechanically coupled to connection features of first fluidic device portion 440, in the form of holes (exemplary connection features 494 of the first fluidic device portion 440 are labeled and described in greater detail in the context of FIG. 12D, below). Of course, it should be understood that while FIG. 12A presents connection features that mechanically couple first fluidic device portion 440 and second fluidic device portion 490, other types of connections may be used and the disclosure is not in this way limited. For example, in some embodiments, first fluidic device portion 440 and second fluidic device portion 490 may be connected using an adhesive. Furthermore, it should be understood that the connection features shown are non-limiting examples, and that any other type of connection feature may be used. For example, first fluidic device portion 440 could instead comprise pegs, while second fluidic device portion 490 could comprise holes, in some embodiments.

As shown, in FIG. 12A, second fluidic device portion 490 may comprise alignment features, which in this case are coupled to alignment features of fluidic receptacle 530. Although the alignment features of second fluidic device portion 490 are obscured in FIG. 12A, they are shown in detail in FIG. 12C, below, and alignment features of fluidic receptacle 530 are shown in FIG. 11K, above. FIG. 12A further shows fluidic connectors 488, which are configured to fluidically connect first fluidic device portion 490 to a channel of a fluidic device (not shown), in some embodiments. For example, fluidic connectors 448 may be connected to the channel via fluidic connections such as fluidic connections 512 shown in FIGS. 11A-11I. In some embodiments, first fluidic device portion 540 comprises a relatively rigid material, as described above. In some embodiments, first fluidic device portion 540 comprises a relatively flexible material, as described above. In certain embodiments, the first fluidic device portion comprises holes 441, through which mounting features may be passed.

FIG. 12B presents fluidic device portions 440 and 490 as in FIG. 12A, oriented in a different perspective, and in the absence of fluidic receptacle 530. Here, an alignment feature 446 is visible, and is analogous to alignment feature 546 of FIGS. 11A-11I. FIG.12C presents the fluidic device portions of FIG. 12B, rotated 180 degrees, such that first alignment feature 404, second alignment feature 406, fifth alignment feature 444, and seventh alignment feature 446 of second fluidic device portion 490 may be seen. Also shown are connecting features 496 of first fluidic device portion 440 which, according to some embodiments, are configured to extend through at least a portion or all of thickness dimension 467 of second fluidic device portion 490. Although not present in every embodiment, and not necessary to align the fluidic receptacle, connection features such as connection features 496 may be configured, in some embodiments, to be received by alignment features of fluidic receptacles such as fluidic receptacle 530, along with alignment features 404, 406, 446, and 444. The presence of connection features such as connection features 496 may, in this way, provide mechanical support to alignment features of second fluidic device portion 490, in some embodiments.

FIG. 12D presents first fluidic device portion 440 in the same orientation in which it appears in FIG. 12C. Second fluidic device portion 490 is not shown in FIG. 12D. Here, connection features 494 (in this exemplary illustration, represented as holes) are shown that are capable of mechanically coupling to connection features 492 of second fluidic device portion 490, in some embodiments. Also shown are fluidic connections 488, previously introduced in FIG. 12A, oriented in FIG. 12D such that ends of fluidic connections 488 not shown in FIG. 12A can be seen. Fluidic connections 488 are configured to extend into the alignment features (404, 406, 444, and 446, shown in FIG. 12C) of second fluidic device portion 490, in some embodiments. According to certain embodiments, fluidic connections 488 do not extend completely through alignment features of second fluidic device portion 490. The fluidic connections may thus fluidically connect the first fluidic device portion to the second fluidic device portion in some embodiments. However, in some embodiments, the fluidic connections 488 may extend completely through second fluidic device portion 490, such that second fluidic device portion 490 is not fluidically connected to first fluidic device portion 488 because no part of second fluidic device 490 can be exposed to fluid transported from first fluidic device 440.

Meanwhile, FIG. 12E presents first fluidic device portion 440 in the same orientation in which it appears in FIG. 12B. Here again, connection features 494 (in this exemplary illustration, represented as holes) are shown that are capable of mechanically coupling to connection features 492 of second fluidic device portion 490, in some embodiments.

FIGS. 12F-12G present second fluidic device portion 490 in the orientations of FIGS. 12B-12C, respectively, without showing first fluidic device portion 440. Here, connection features 492 and alignment features 404, 406, 444, and 446 are shown, as described above. Also shown are connection features 498, which in this case are shown as holes configured to receive connection features 496 described above.

FIG. 12H presents a schematic cross-section of exemplary fluidic device portions 440 and 490, according to some embodiments. This cross-section illustrates the degree to which fluidic connections 488 of first fluidic device portion 440 extend into alignment features 404 and 444 of second fluidic device portion 490 in this non-limiting, exemplary embodiment. As shown, in this embodiment, first fluidic device portion 440 is fluidically connected to second fluidic device portion 490, since fluid can contact both fluidic device portions as it is transmitted through alignment feature 404.

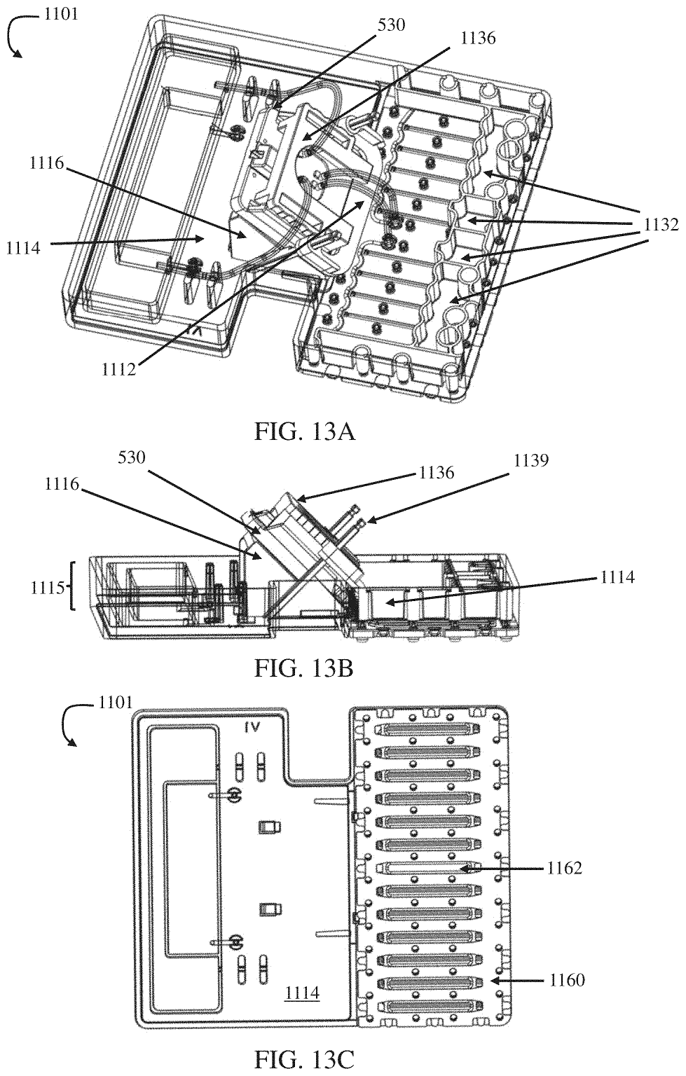

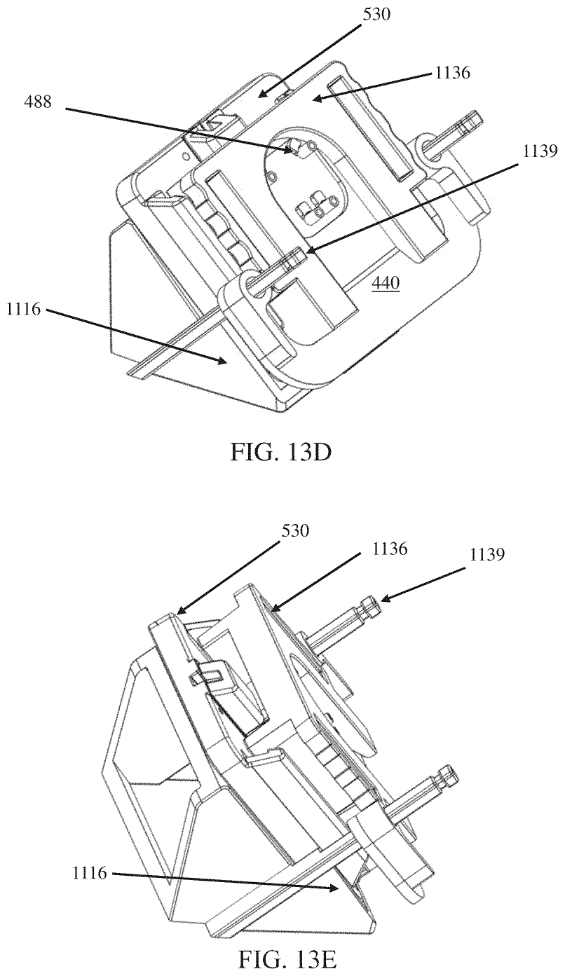

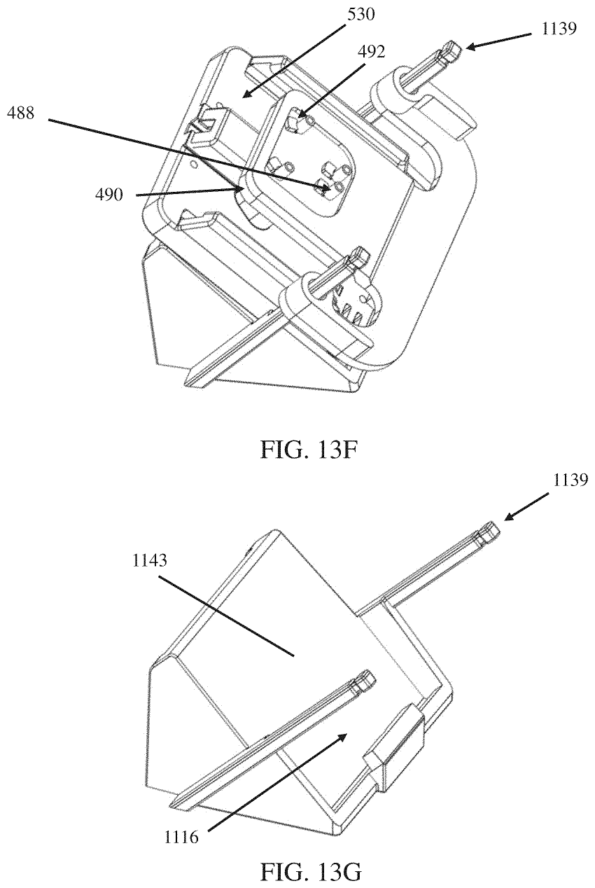

FIGS. 13A-13G show various views of a schematic illustration of another fluidic device for transferring fluid to a fluidic receptacle in the form of an integrated device, according to some embodiments. FIG. 13A shows a perspective schematic illustration of fluidic device 1101 and fluidic receptacle 530 shown in greater detail in FIGS. 11K-11L above, while FIG. 13B shows a side-view schematic illustration of fluidic device 1101 and fluidic receptacle 530, according to some embodiments. FIG. 13C shows a bottom-view schematic illustration of fluidic device 1101, according to some embodiments. Fluidic device 1101, in the exemplary embodiments illustrated in FIGS. 13A-13G, comprises cartridge 1115, solid substrate 1114, and mounting element 1116. In the example of FIGS. 13A-13G, cartridge 1115 was prepared by injection molding. In the exemplary embodiment of FIGS. 13A-13G, mounting element 1116 is in a raised portion of fluidic device 1101. In some embodiments, mounting element 1116 further comprises mounting features 1139 (illustrated in FIGS. 13A-13G as posts of the mounting element, configured to extend through holes 441 of fluidic device portion 440, which is shown in greater detail in the illustrations of the embodiment shown in FIGS. 12A-12E).

Fluidic device 1101, according to some embodiments, is configured to receive a fluidic receptacle 530 at mounting element 1116. Further, in some embodiments, fluidic device receptacle 530 is configured to couple to alignment features 524, 526, 554, and 556, such that fluidic receptacle 530 is fluidically connected to the fluidic device via fluidic connections 1112. In some embodiments, fluidic device 1101 comprises reservoirs 1132, which may contain samples or reagents, and which are fluidically connected to fluidic receptacle 530 via fluidic connections 1112. In some embodiments, fluidic device 1101 further comprises a mechanical coupler 1136, which can mechanically support the connection between fluidic receptacle 530 and discrete fluidic device portions 440 and 490. In some embodiments, device 1101 further comprises first fluidic device portion 440 and second fluidic device portion 490 as shown in FIGS. 12A-12H. In some embodiments, fluidic device 1101 comprises seal plate 1160 and elastomer surface 1162.

FIGS. 13D-13E show schematic perspective illustrations of mounting element 1116, fluidic receptacle 530, first fluidic device portion 440, which is connected to second fluidic device portion 490, and mechanical coupler 1136, according to some embodiments. FIG. 13F shows a schematic, perspective illustration of the system without the mechanical coupler, revealing second fluidic device portion 490, according to some embodiments. Finally, FIG. 13G provides a schematic perspective illustration of mounting element 1116, comprising mounting features 1139 and surface 1143 that is angled with respect to a lateral dimension of fluidic device 1101, according to certain embodiments.

FIGS. 14A-14B present various schematic perspective illustrations of another exemplary embodiment of first discrete fluidic device portion 440 and second discrete fluidic device portion 490. In these embodiments, second discrete fluidic device portion 490 has been over-molded onto first fluidic device portion 490. As with the discrete fluidic device portions shown in FIGS. 12A-12H, these fluidic device portions can couple to an exemplary fluidic receptacle of the type illustrated in FIGS. 11K-11L. FIG. 14C presents a truncated version of the fluidic device portions, illustrating that the first fluidic device portion 440 does not extend into alignment features 404, 406, 444, and 446 of second fluidic device portion 490. However, fluidic device portions 440 and 490 are fluidically connected, and are configured such that fluid passing through an alignment feature will also pass through a fluidic connector 488, as shown in FIG. 14C. Examples of benefits of this type of over-molded second discrete fluidic device portion are described in greater detail above.

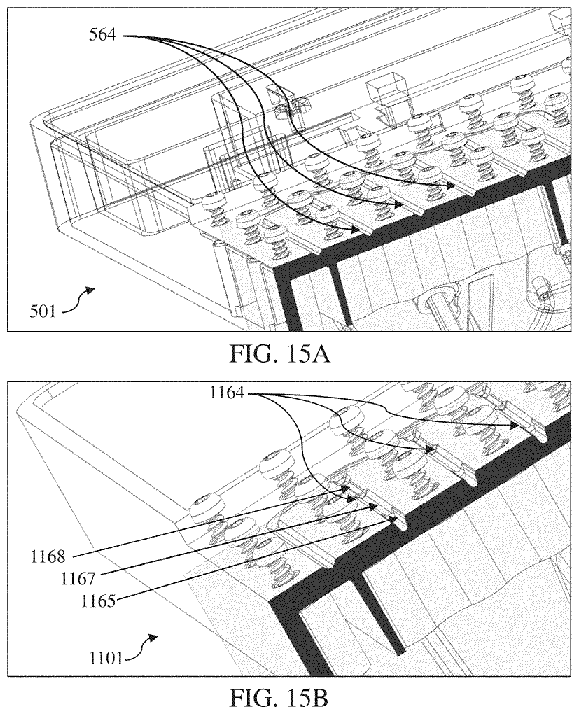

FIGS. 15A and 15B present perspective, schematic diagrams of channels of a cross-cut fluidic device, according to various embodiments. FIG. 15A illustrates channels 564 of fluidic device 501, also shown in FIG. 11G. As shown, channels 564 have uniform depth. FIG. 15B presents channels 1164 of fluidic device 1101 (see FIGS. 13A-13G). Channels 1164 are arranged similarly to channels 564, but have a variable depth, as shown. Thus, channels 1164 comprise relatively deep channel portions 1165 and relatively shallow channel portions 1167, as well as additional portions 1168. Channel portions 1165 and 1167 have an identical average diameter, while channel portion 1168 has a smaller average diameter. Although channels 564 are shown with reference to device 501 and channels 1164 are shown with reference to device 1101, it should be understood that constant or variable-depth channels may be used in connection with either type of fluidic device, and the disclosure is not so limited.

The following applications are incorporated herein by reference, in their entirety, for all purposes: U.S. Patent Publication No. US-2021-0121874-A1, published April 29, originally filed as U.S. application Ser. No. 17/083,106 on Oct. 28, 2020, entitled “PERISTALTIC PUMPING OF FLUIDS AND ASSOCIATED METHODS, SYSTEMS, AND DEVICES”; U.S. Patent Publication No. US-2021-0121875-A1, published Apr. 29, 2021, originally filed as U.S. application Ser. No. 17/083,126 on Oct. 28, 2020, entitled “PERISTALTIC PUMPING OF FLUIDS FOR BIOANALYTICAL APPLICATIONS AND ASSOCIATED METHODS, SYSTEMS, AND DEVICES”; U.S. Patent Publication No. US-2021-0121879-A1, published Apr. 29, 2021, originally filed as U.S. patent application Ser. No. 17/082,223 on Oct. 28, 2020, entitled “SYSTEMS AND METHODS FOR SAMPLE PREPARATION”; U.S. Patent Publication No. US-2021-0164035-A1, published Jun. 3, 2021, originally filed as U.S. patent application Ser. No. 17/082,226 on Oct. 28, 2020, entitled “METHODS AND DEVICES FOR SEQUENCING”; and U.S. Patent Publication No. US-2021-0221839-A1, published Jul. 22, 2021, originally filed as U.S. patent application Ser. No. 17/153,490 on Jan. 20, 2021, entitled “COMPOUNDS AND METHODS FOR SELECTIVE C-TERMINAL LABELING”.

U.S. Provisional Application No. 63/177,882, filed Apr. 21, 2021, and entitled “DEVICES AND METHODS FOR LOADING OF FLUIDIC RECEPTACLES,” and U.S. Provisional Application No. 63/271,944, filed Oct. 26, 2021, and entitled “DEVICES AND METHODS FOR LOADING OF FLUIDIC RECEPTACLES,” are incorporated herein by reference in their entirety for all purposes.

### Exemplary Workflow 1

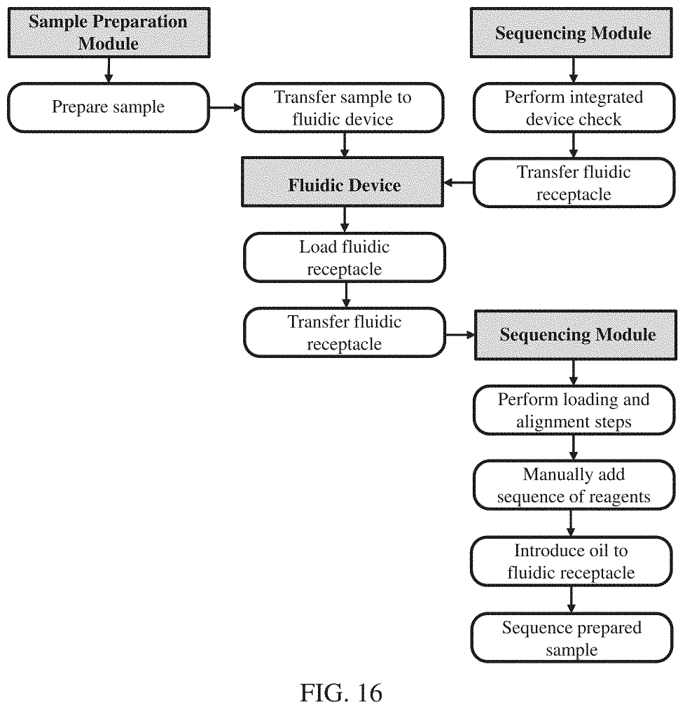

This section describes an exemplary workflow for preparing a sample, loading the sample using a fluidic device, and sequencing the sample, according to some embodiments. The workflow is presented in FIG. 16. First, the sample is prepared using a sample preparation module, and a fluidic receptacle of the type illustrated in FIGS. 11K-11L is placed in the sequencing module, where an integrated device check is performed.

Next, the fluidic receptacle is transferred to recess 516 of fluidic device 501 as illustrated in FIGS. 11A-11F, which is configured to receive the fluidic receptacle in an alignment. Meanwhile, the prepared sample is loaded into the fluidic receptacle through the coupling between first alignment feature 504 and third alignment feature 524; and/or through the coupling between fifth alignment feature 544 and sixth alignment feature 554, as illustrated in FIGS. 11A-11N, which are positioned below outlets 520 with respect to gravity, such that bubbles from the quantity of fluid will preferentially travel towards outlets 520. After the transfer step, the fluid in the fluidic receptacle is mixed by translating a roller across the surface of microchannels fluidically connected to outlets 520 while applying a constant pressure to the surface, and subsequently translating the roller across the surface in the opposite direction while applying same pressure. This process mixes the liquid within the fluidic receptacle, without producing a net flow of fluid through outlets 520 during mixing.

Once the transfer process is complete, the fluidic receptacle is removed from the flow cell and returned to the sequencing module, where loading and alignment steps are performed. Next, a sequence of reagents is manually added to prepare the fluidic receptacle for sequencing. Finally, an oil is manually introduced to cover portions of the fluidic receptacle, and the prepared sample is sequenced in the sequencing module.

### Exemplary Workflow 2

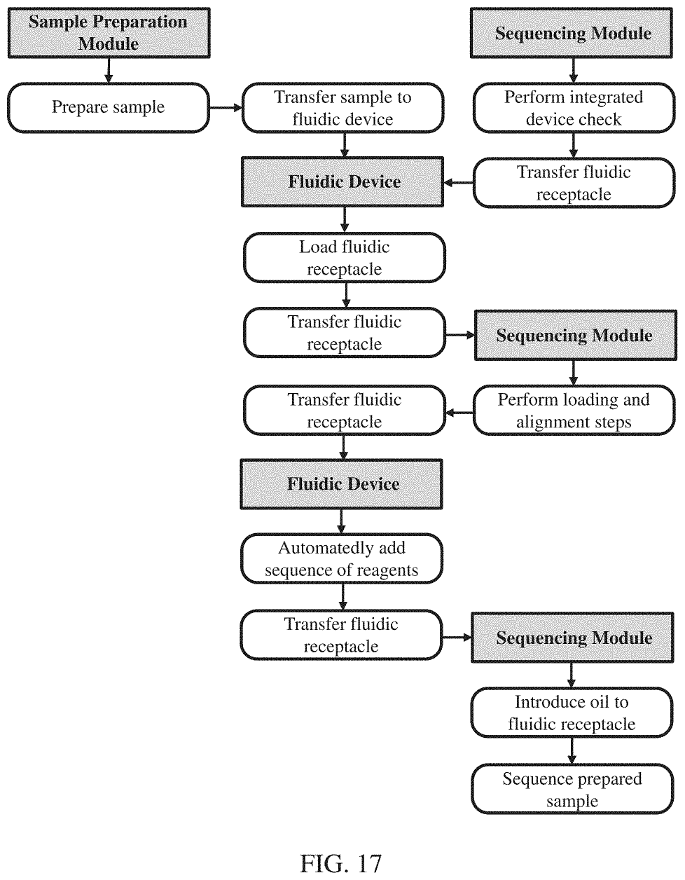

This section describes an exemplary workflow for preparing a sample, loading the sample using a fluidic device, and sequencing the sample, according to some embodiments. The workflow is presented in FIG. 17. First, the sample is prepared using a sample preparation module, and a fluidic receptacle of the type illustrated in FIGS. 11K-11L is placed in the sequencing module, where an integrated device check is performed.

Next, the fluidic receptacle is transferred to recess 516 of fluidic device 501 as illustrated in FIGS. 11A-11F, which is configured to receive the fluidic receptacle in an alignment. Meanwhile, the prepared sample is loaded into the fluidic receptacle through the coupling between first alignment feature 504 and third alignment feature 524; and/or through the coupling between fifth alignment feature 544 and sixth alignment feature 554, as illustrated in FIGS. 11A-11N, which are positioned below outlets 520 with respect to gravity, such that bubbles from the quantity of fluid will preferentially travel towards outlets 520. After the transfer step, the fluid in the fluidic receptacle is mixed by translating a roller across the surface of microchannels fluidically connected to outlets 520 while applying a constant pressure to the surface, and subsequently translating the roller across the surface in the opposite direction while applying same pressure. This process mixes the liquid within the fluidic receptacle, without producing a net flow of fluid through outlets 520 during mixing.

Once the transfer process is complete, the fluidic receptacle is removed from the flow cell and returned to the sequencing module, where loading and alignment steps are performed. Next, the fluidic receptacle is transferred back to fluidic device 501, where a sequence of reagents is automatedly added to prepare the fluidic receptacle for sequencing. Finally, an oil is manually introduced to cover portions of the fluidic receptacle, and the fluidic receptacle is transported back to the sequencing module, where an alignment step is performed and the sample is sequenced.

### Exemplary Workflow 3

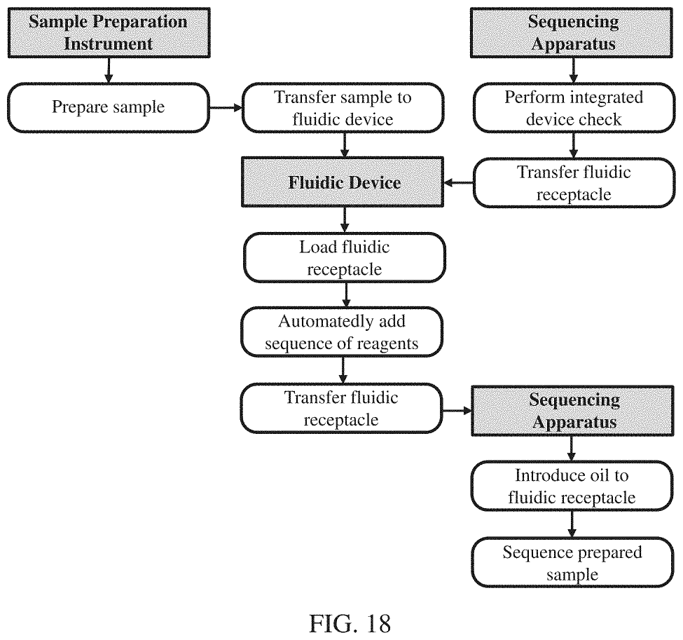

This section describes an exemplary workflow for preparing a sample, loading the sample using a fluidic device, and sequencing the sample, according to some embodiments. The workflow is presented in FIG. 18. First, the sample is prepared using a sample preparation module, and a fluidic receptacle of the type illustrated in FIGS. 11K-11L is placed in the sequencing module, where an integrated device check is performed.

Next, the fluidic receptacle is transferred to recess 516 of fluidic device 501 as illustrated in FIGS. 11A-11F, which is configured to receive the fluidic receptacle in an alignment. Meanwhile, the prepared sample is loaded into the fluidic receptacle through the coupling between first alignment feature 504 and third alignment feature 524; and/or through the coupling between fifth alignment feature 544 and sixth alignment feature 554, as illustrated in FIGS. 11A-11N, which are positioned below outlets 520 with respect to gravity, such that bubbles from the quantity of fluid will preferentially travel towards outlets 520. After the transfer step, the fluid in the fluidic receptacle is mixed by translating a roller across the surface of microchannels fluidically connected to outlets 520 while applying a constant pressure to the surface, and subsequently translating the roller across the surface in the opposite direction while applying same pressure. This process mixes the liquid within the fluidic receptacle, without producing a net flow of fluid through outlets 520 during mixing.

Once the transfer process is complete, a sequence of reagents is automatedly added to prepare the fluidic receptacle for sequencing. Finally, an oil is manually introduced to cover portions of the fluidic receptacle, and the fluidic receptacle is transported back to the sequencing module, where an alignment step is performed and the sample is sequenced.

The following examples are intended to illustrate some embodiments of the present invention, but do not exemplify the full scope of the invention.

### EXAMPLE 1

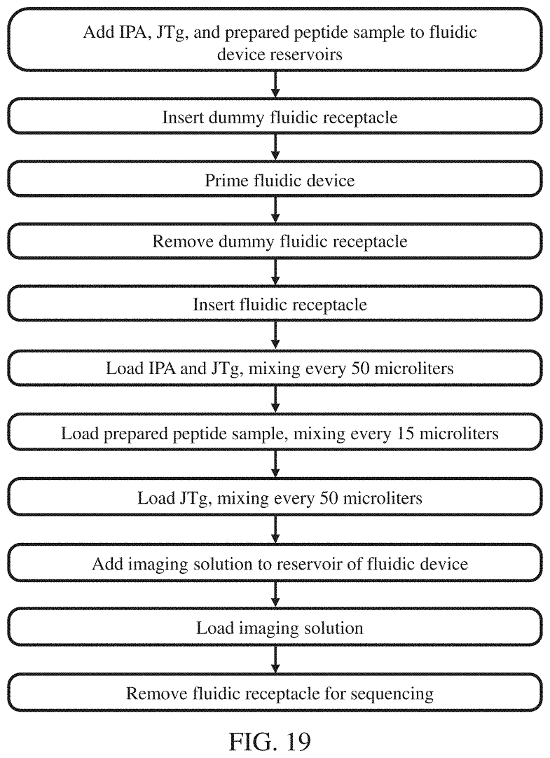

This example describes a method used to load a prepared peptide sample to a fluidic receptacle using a fluidic device, and for preparing the fluidic receptacle for sequencing the sample, according to some embodiments. The method is presented in FIG. 19. First, isopropyl alcohol (IPA) and JT buffer (JTg), and a prepared peptide sample were added to reservoirs of an exemplary fluidic device 501 of the type illustrated in FIGS. 11K-11L. Next, a ‘dummy’ fluidic receptacle was transferred to recess 516 of fluidic device 501 as illustrated in FIGS. 11A-11F, and the fluidic device was primed, such that fluidic connections 512 were filled with fluid.

Once the priming was complete, the dummy fluidic receptacle was removed and the fluidic receptacle was added. IPA and JTg were loaded into the fluidic receptacle by peristaltically pumping 50 microliters of fluid into the fluidic receptacle. The fluid in the fluidic receptacle was then mixed by translating a roller across the surface of microchannels fluidically connected to outlets 520 while applying a constant pressure to the surface, and subsequently translating the roller across the surface in the opposite direction while applying same pressure. By performing repeating these steps of fluid transfer and mixing, the IPA and the JTg were loaded into the fluidic receptacle and used to wash the fluidic receptacle.

Next, the prepared peptide sample was loaded into the fluidic receptacle by peristaltically pumping 15 microliters of fluid into the fluidic receptacle. The fluid in the fluidic receptacle was then mixed by translating a roller across the surface of microchannels fluidically connected to outlets 520 while applying a constant pressure to the surface, and subsequently translating the roller across the surface in the opposite direction while applying same pressure. By performing repeating these steps of fluid transfer and mixing, the prepared sample was loaded into the fluidic receptacle. Once loaded, the prepared sample was incubated for 20-30 minutes.

After incubation, JTg was loaded into the fluidic receptacle by repeatedly performing steps of peristaltically pumping 50 microliters of fluid into the fluidic receptacle, followed by mixing the fluid in the fluidic receptacle. This process washed away excess peptide, including peptide that non-specifically bound to the fluidic receptacle. As a result, the peptide remaining within the fluidic receptacle was bound to the fluidic receptacle.

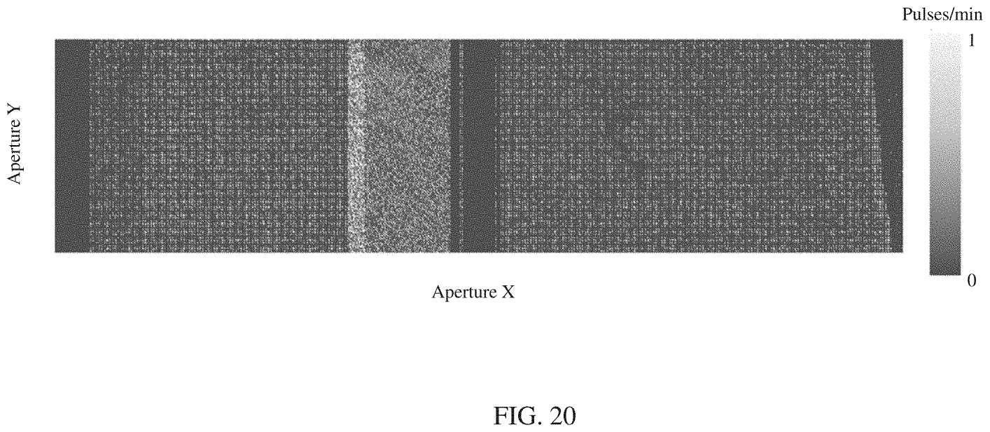

Finally, an imaging solution was added to a reservoir of fluidic device 501, and was flowed through the fluidic receptacle, which was subsequently removed from recess 516 and transferred to a sequencing module for analysis. The sequencing analysis resulted in the homogeneous cumulative recognition pulsing activity profile shown in FIG. 20 over the course of the run, indicating that sample loading using the fluidic device was successful.

### EXAMPLE 2

This example describes the flow of dye, dissolved in IPA, as it passes through a fluidic device. First, IPA containing red dye was added to a first reservoir of a fluidic device, while JTg was added to a second reservoir. The IPA was loaded into the fluidic device, such that fluidic connections to both an inlet and an outlet of a fluidic receptacle coupled to the fluidic device had a red color. Then, JTg was peristaltically pumped through the fluidic connection to the inlet of the fluidic receptacle, to the outlet of the fluidic receptacle, and to a waste outlet of the fluidic device. As the JTg, a clear fluid, was pumped into the fluidic device, the red discoloration slowly cleared. A color gradient between the IPA and the JTg indicates that some mixing occurred in the fluidic receptacle—however, the ultimate, clear color of the fluid indicates that almost all of the dyed IPA cleared the fluidic receptacle. No leakage or flow issues were observed. This example demonstrates that the fluidic devices described operate as intended.

### EXAMPLE 3

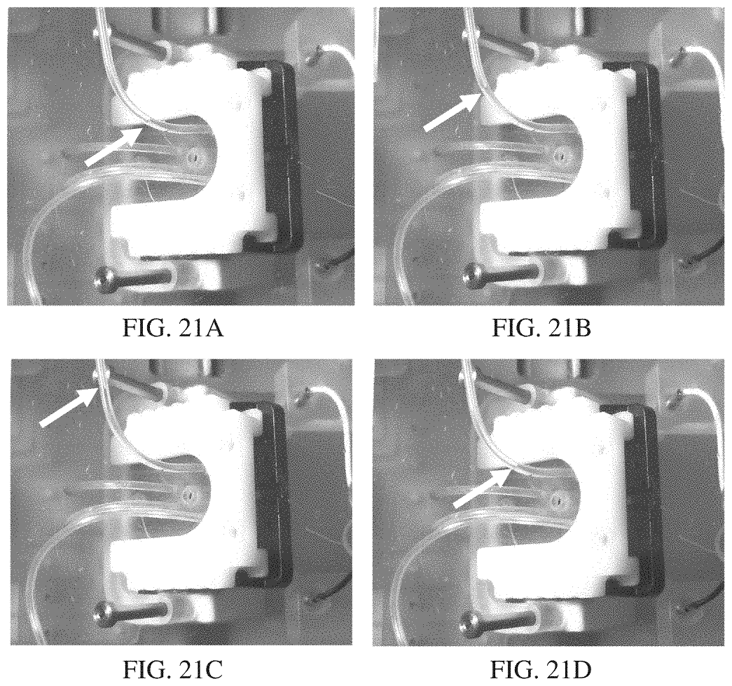

This example demonstrates the process of mixing fluid in a fluidic receptacle. In this example, the fluidic device contains two layers of fluid: a layer of liquid comprising yellow dye, and a layer of air. These layers are shown in FIGS. 21A-21D, which present photographs of the fluidic device at various time-points during the mixing. In FIGS. 21A-21D, the interface between the liquid layer and the air layer is indicated by the white arrow. First, a roller was translated across a surface of a microchannel (not shown) containing a quantity of fluid in a first direction while applying pressure to the surface. This pumped fluid through the fluidic receptacle, moving the interface from its position in FIG. 21A to its position in FIG. 21B, and ultimately to its position in FIG. 21C. Next, the roller was translated across the surface in the opposite direction while applying pressure to at least a portion of the surface. This pumped fluid through the fluidic receptacle in the opposite direction. The process was continued until the interface reached its position in FIG. 21D, matching its original position at the time at its original time-point, as photographed in FIG. 21A. Through the iteration of this process, fluid in the fluidic receptacle was mixed without producing a net flow through the fluidic receptacle.

### EXAMPLE 4

This example demonstrates the use of an exemplary fluidic device of the type presented in FIGS. 12A-12H to reliably loading of an exemplary integrated device with homogeneity and loading comparable to a manual loading process. In this example, eight autoloading and sequencing runs were performed, during which one of four exemplary peptide samples, hereafter referred to as Sample 1, Sample 2, Sample 3, and Sample 4, was loaded onto one of eight exemplary integrated devices, hereafter referred to as Integrated Device 1, Integrated Device 2, Integrated Device 3, Integrated Device 4, Integrated Device 5, Integrated Device 6, Integrated Device 7, and Integrated Device 8, using the fluidic device presented in FIGS. 12A-12H. Each exemplary integrated device was then loaded with the same peptide by hand, and each sample in its respective was sequenced to determine a loading percentage—a percentage of wells of the integrated device that comprised a labeled peptide. The loading percentage of a peptide can be an important consideration for sequencing a peptide, so a high loading percentage is advantageous for sequencing applications, in some embodiments.

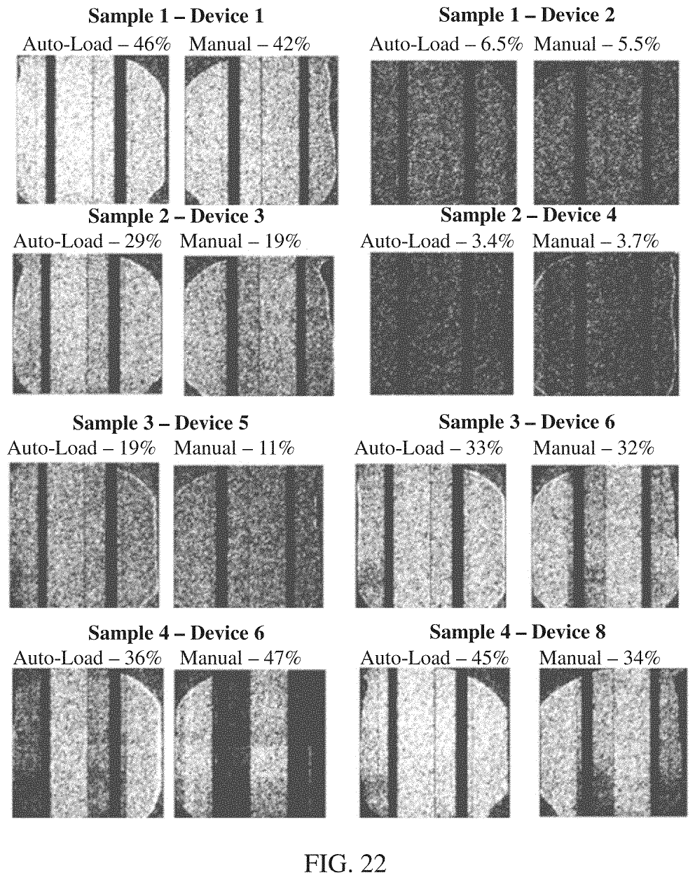

Sequencing and preparation was performed as explained in Example 1, in order to compare reliability of the autoloading with respect to the manual loading process. Of course, it should be understood that in this example, sequencing is used as a test of the loading process, and that the loading process may be appropriate for any sequencing or preparation process. FIG. 22 shows cumulative recognition pulsing activity profiles associated with sequencing of the integrated devices loaded using the exemplary fluidic device (“Auto-Load”) and integrated devices loaded manually (“Manual”), collected over the course of each experiment. In general, the automated loading of integrated devices produced more uniform profiles, demonstrating the improved performance associated with use of the fluidic device. FIG. 22 further indicates the loading percentage of each sample/integrated device combination, demonstrating that generally the loading percentage increased during auto-loading of the integrated device. These experiments indicate that automatic loading of an integrated device using fluidic devices, systems, and methods such as those described herein can advantageously improve sample loading, in some embodiments.

### EXAMPLE 5

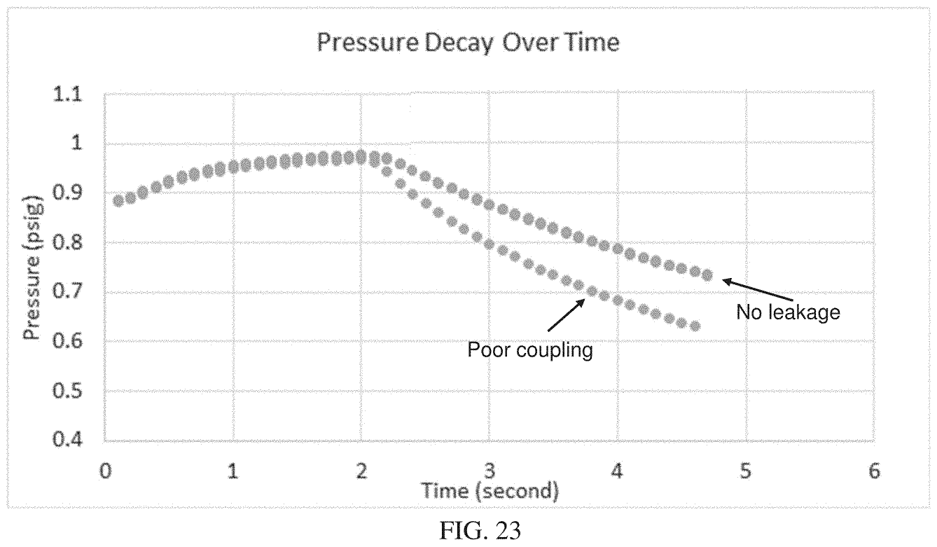

This example describes leak testing of an exemplary fluidic-receptacle, fluidically coupled to an exemplary fluidic device comprising a single fluidic device portion. The fluidic device and fluidic receptacle design are illustrated in FIGS. 11A-11I above. In the leak testing, the fluidic receptacle was pressurized to a target pressure, the pressure was allowed to stabilize for a short period of time, and then the pressure of the exemplary fluidic device was measured over time to determine a relative rate of leakage. In this example, the fluidic device was coupled to and uncoupled from the fluidic receptacle 3 times. Each time, it was assembled with the same fluidic device and fluidic receptacle for a leak testing run (shown as solid points). In a separate experiment, leakage from the cartridge, pressurized and decoupled from the fluidic receptacle, was measured for comparison. FIG. 23 presents the pressure decay resulting from these experiments. Two leak testing runs overlapped nearly perfectly with the pressure decay from the fluidic device (not shown), indicating no leakage from the connection to the fluidic receptacle, while one experiment demonstrated more rapid leakage, indicating that the coupling between the fluidic device and the fluidic receptacle sealed poorly.

### EXAMPLE 6

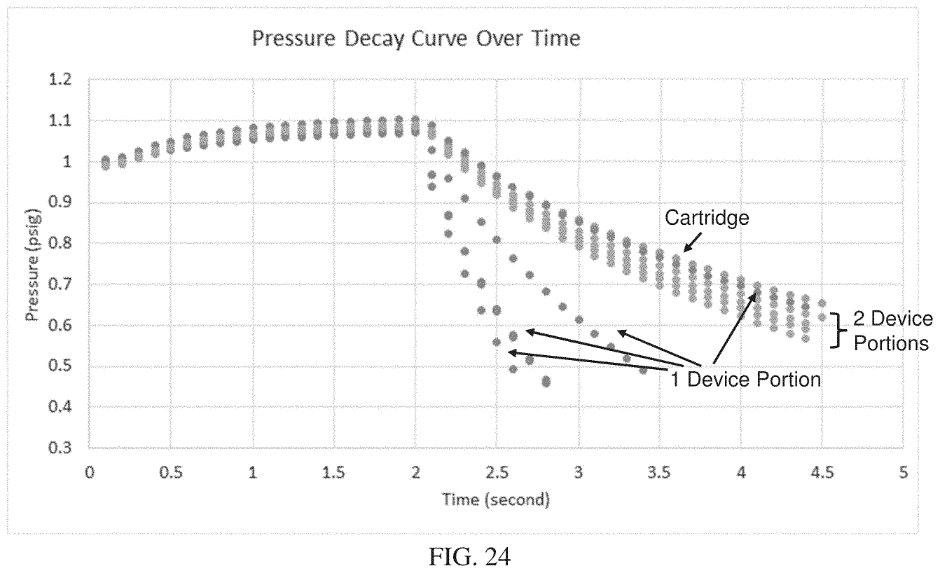

This example describes a leak test comparison between the exemplary fluidic device of Example 5 with the exemplary device illustrated in FIGS. 12A-12H, which comprises a first, relatively rigid fluidic device portion and a second, relatively soft fluidic device portion. Using the same protocol, leakage from each fluidic device was measured. As shown in FIG. 24, plotting the pressure decay rate over time for 5 cycles of decoupling and recoupling the fluidic receptacle to the fluidic device, using multiple fluidic device portions produced more reliable results with less overall leakage than use of a single fluidic device portion. This result demonstrates the advantages which can be, in some embodiments, associated with using multiple fluidic device portions.

### EXAMPLE 7

This example demonstrates the use of an exemplary fluidic device of the type presented in FIGS. 12A-12H to reliably load a variety of integrated devices with an exemplary peptide sample, hereafter referred to as Sample 5. In this example, Sample 5 was loaded onto one of 41 exemplary integrated devices, hereafter referred to as Integrated Devices 9-49, using the fluidic device presented in FIGS. 12A-12H. Each exemplary integrated device was then loaded with the same peptide by hand, and each sample was sequenced to determine a loading fraction—a fraction of wells of the integrated device that comprised a labeled peptide. The loading fraction is related to the loading percentage of Example 4 above. Generally, the loading percentage is the loading fraction multiplied by 100%. The loading fraction of a peptide can be an important consideration for sequencing a peptide, so a high loading fraction is advantageous, in some embodiments.

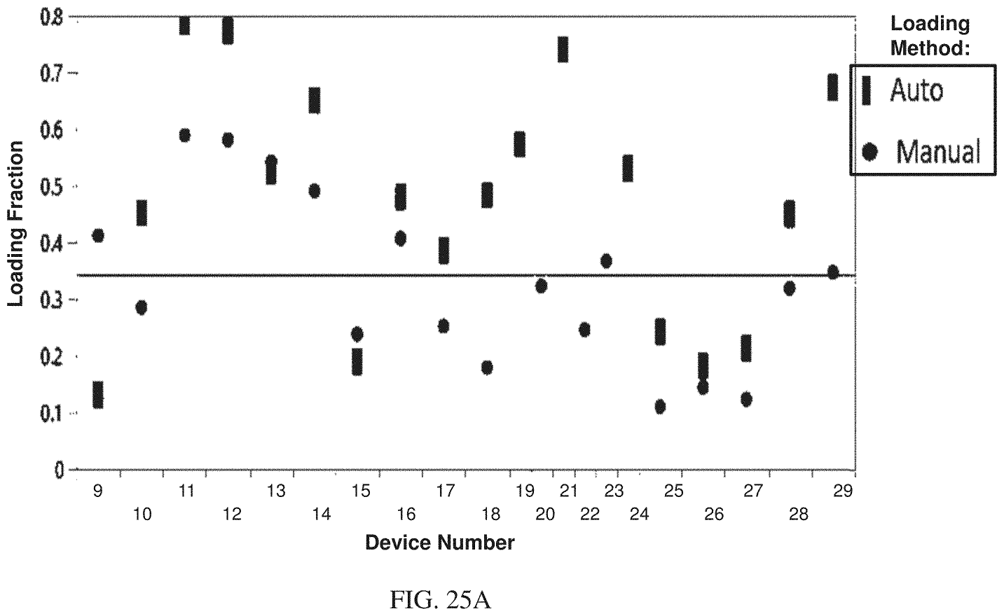

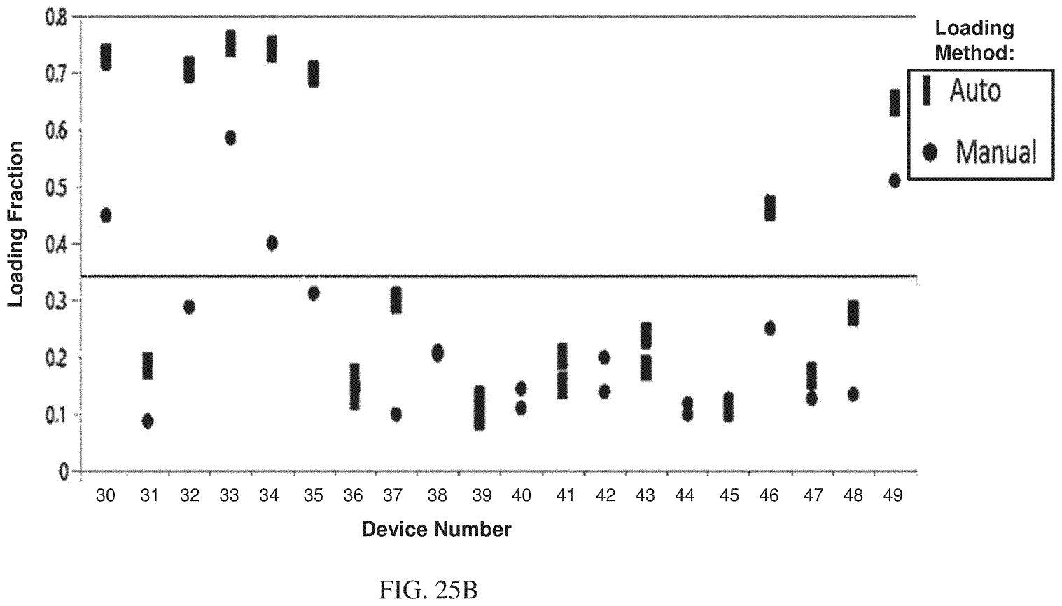

Sequencing and preparation was performed as explained in Example 1, in order to compare reliability of the autoloading with respect to the manual loading process. Of course, it should be understood that in this example, sequencing is used as a test of the loading process, and that the loading process may be appropriate for any sequencing or preparation process. FIGS. 25A-25B show the loading fraction of the integrated devices loaded using the exemplary fluidic device (“Auto”) and integrated devices loaded manually (“Manual”), collected over the course of each experiment. In general, the automated loading of integrated devices produced loading fractions that equaled or exceeded the manual loading of the integrated devices, demonstrating the improved performance associated with use of the fluidic device.

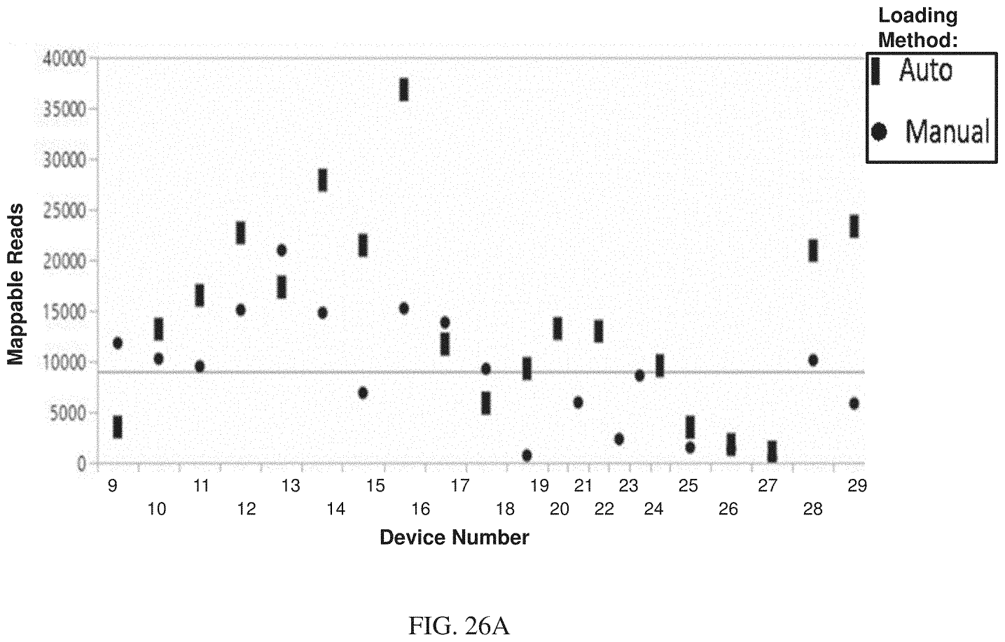

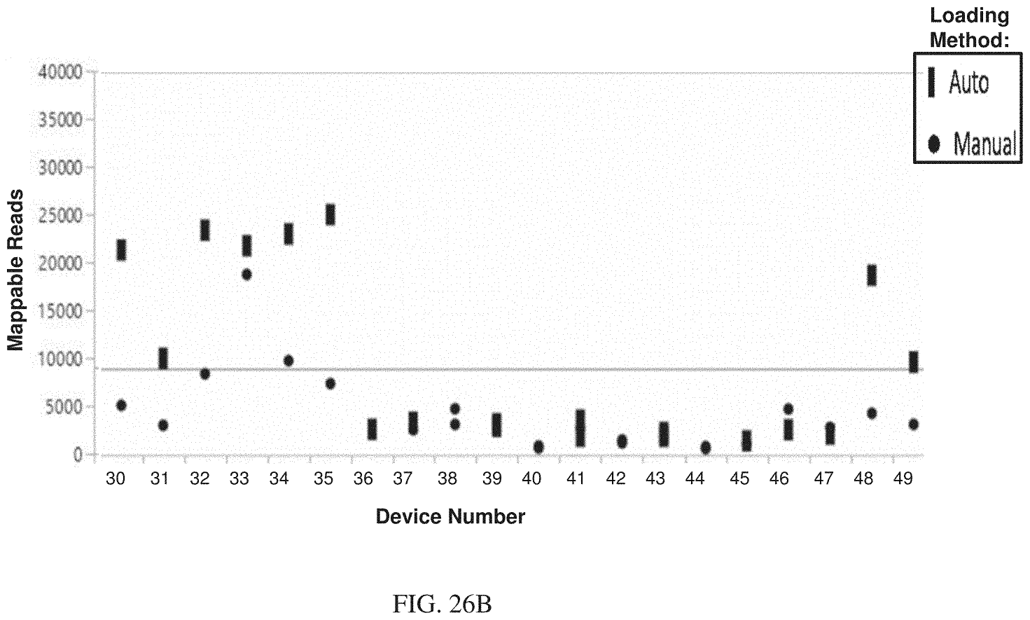

FIGS. 26A-26B show the number of mappable reads of the integrated devices loaded using the exemplary fluidic device (“Auto”) and integrated devices loaded manually (“Manual”), collected over the course of each experiment. In general, the automated loading of integrated devices produced a number of mappable reads that equaled or exceeded the number of mappable reads of the integrated devices, demonstrating the improved performance associated with use of the fluidic device. In some embodiments, a high number of mappable reads is advantageous for sequencing applications.

These experiments indicate that automatic loading of an integrated device using fluidic devices, systems, and methods such as those described herein can advantageously improve sample loading, in some embodiments.

### EXAMPLE 8

This example demonstrates that surface chemistry can play a role in the loading of integrated devices. In this example, an integrated device, hereafter referred to as Integrated Device 50, was loaded with an exemplary peptide sample, hereafter referred to as Sample 6. The integrated device was given a surface treatment (one of treatments hereafter referred to as Treatment 1, Treatment 2, or Treatment 3). The integrated device was then auto-loaded or loaded manually, according to the procedure of Example 4, above.

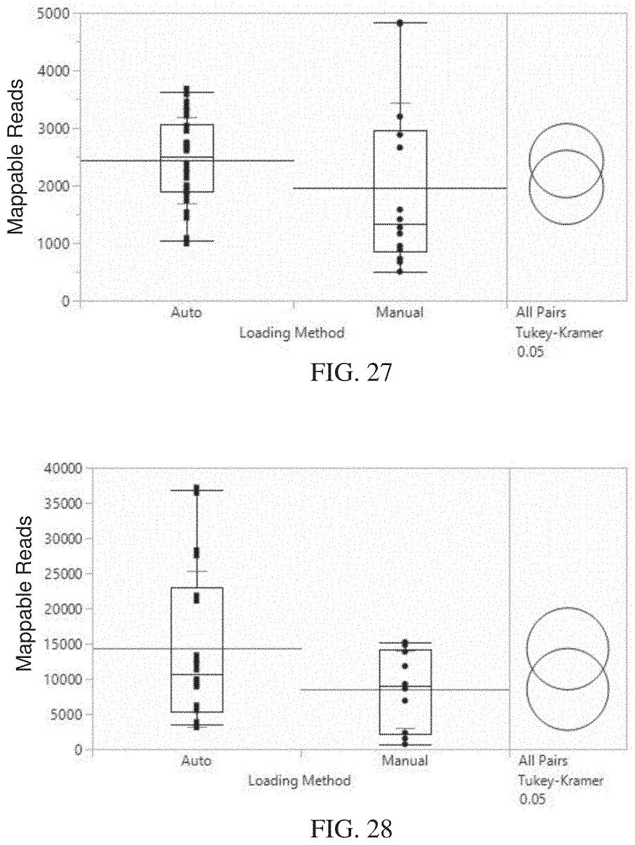

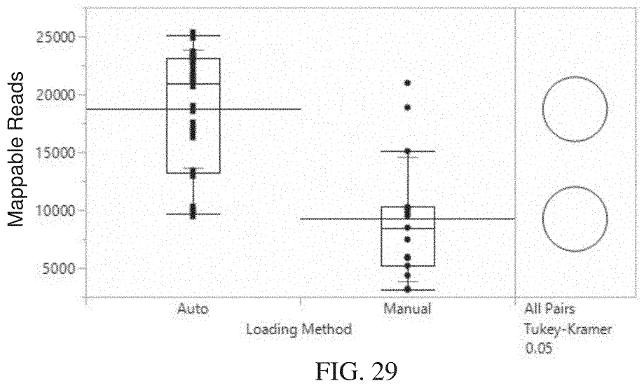

The experiment was repeated 14 times for each loading method and surface treatment. FIG. 27 presents the number of mappable reads of Integrated Device 50 after receiving Treatment 1. FIG. 28 presents the number of mappable reads of Integrated Device 50 after receiving Treatment 2. FIG. 29 presents the number of mappable reads of Integrated Device 50 after receiving Treatment 3. Treatment 1 and Treatment 2 showed no statistically significant difference between manual loading and auto-loading, although auto-loading generally resulted in improved loading. As shown in FIGS. 27-29, the number of mappable reads depended strongly on treatment. In the case of Treatment 3 (FIG. 29), auto-loading produced a statistically significantly higher number of mappable reads than manual loading.

These results demonstrate that surface chemistry is important for loading of integrated devices, and show that statistically significant improvements in loading may result from auto-loading, rather than manual loading, of integrated devices.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A fluidic system for transferring fluid to a fluidic receptacle, comprising:
a fluidic device, comprising:
a first alignment feature,
a channel that is fluidically connected to the first alignment feature, and
a second alignment feature;
wherein:
the fluidic device is configured to receive the fluidic receptacle in an alignment with respect to the fluidic device via a first coupling between the first alignment feature and a third alignment feature, located on the fluidic receptacle, and via a second coupling between the second alignment feature and a fourth alignment feature, located on the fluidic receptacle, such that the channel is fluidically connected to the fluidic receptacle through the first alignment feature and the second alignment feature.

2. The fluidic system of claim 1, wherein the fluidic device is configured to be fluidically connected to the fluidic receptacle via a coupling between a fifth alignment feature, located on the fluidic device, and a sixth alignment feature, located on the fluidic receptacle.

3. The fluidic system of claim 1, wherein the fluidic device comprises a recess comprising a surface, angled with respect to a lateral dimension of the fluidic device, configured to receive the fluidic receptacle.

4. The fluidic system of claim 1, wherein the angle between the surface and the lateral dimension is greater than or equal to 10° and less than or equal to 80°.

5. (canceled)

6. The fluidic system of claim 1, further comprising the fluidic receptacle.

7. The fluidic system of claim 6, wherein the fluidic receptacle comprises or contains an integrated device.

8-10. (canceled)

11. The fluidic system of claim 7, wherein the integrated device comprises at least one component configured to generate a radiation pattern based on emission energy emitted from a sample.

12-13. (canceled)

14. The fluidic system of claim 1, wherein the fluidic device comprises a cartridge.

15. The fluidic system of claim 14, wherein the cartridge comprises a base layer having a surface comprising channels.

16-19. (canceled)

20. The fluidic system of claim 1, configured to receive an intervening part between the first alignment feature and the third alignment feature.

21. (canceled)

22. The fluidic system of claim 20, wherein the intervening part is a gasket or molded layer.

23. The fluidic system of claim 1, wherein the fluidic device comprises a discrete fluidic device portion.

24. The fluidic system of claim 23, wherein the first alignment feature and/or the second alignment feature are disposed on the discrete fluidic device portion.

25. The fluidic system of claim 1, wherein the fluidic device comprises a first discrete fluidic device portion and a second discrete fluidic device portion.

26. The fluidic system of claim 25, wherein the channel is fluidically connected to the second discrete fluidic device portion via the first fluidic device portion, and wherein the first alignment feature and/or the second alignment feature are disposed on the second discrete fluidic device portion.

27. The fluidic system of claim 25, wherein the first fluidic device portion comprises a relatively rigid material and the second fluidic device portion comprises a relatively flexible material, and a ratio between an average elastic modulus of the relatively rigid material and an average elastic modulus of the relatively flexible material is greater than or equal to 2.

28. (canceled)

29. The fluidic system of claim 1, wherein the channel comprises a first, shallow portion across the length of the channel and a second, deep portion across the length of the channel.

30. A method, comprising:
coupling a fluidic receptacle to two or more alignment features;
transferring a fluid to the fluidic receptacle via at least one of the alignment features; and
decoupling the fluidic receptacle from the alignment features.

31. (canceled)

32. The method of claim 30, further comprising sequencing a species present in the fluid.

33-92. (canceled)

93. A method of making a fluidic device, the method comprising:
assembling a surface article comprising a surface layer with a base layer to form a cartridge of the fluidic device, wherein:
the surface layer comprises an elastomer, and
the base layer comprises one or more channels; and
fluidically connecting at least one of the channels to a discrete fluidic device portion comprising an alignment feature.

94-100. (canceled)
